SEQUENCE LISTING (1 sequence)

SEQ ID NO: 1 - protein, 8 aa, synthetic construct
GPLGIAGQ

(19) United States
(12) Patent Application Publication
(10) Pub. No.: US 2025/0387494 A1
(43) Pub. Date: Dec. 25, 2025

(54) Prodrugs Based On Tumor Targeting Near Infrared Dye-Drug Conjugates (DDC)

(71) Applicant: Lahjavida, Inc., Colorado Springs, CO (US)

(72) Inventors: Lyle D. Small, Colorado Springs, CO (US); Ruizheng Wang, Colorado Springs, CO (US)

(73) Assignee: Lahjavida, Inc., Colorado Springs, CO (US)

(21) Appl. No.: 19/035,666

(22) Filed: Jan. 23, 2025

Related U.S. Application Data
(63) Continuation-in-part of application No. 18/869,200, filed on Nov. 25, 2024.
(60) Provisional application No. 63/344,972, filed on May 23, 2022.
(60) Provisional application No. 63/624,122, filed on Jan. 23, 2024.

(51) Int. Cl. A61K 47/54

(52) U.S. Cl. CPC A61K 47/558

(57) ABSTRACT

Provided herein are dye-drug conjugate (DDC) compounds comprising: a tumor-targeting near-infrared dye; a chemotherapeutic drug; and a cleavable linker, and methods of use thereof.

### SEQUENCE LISTING

This application contains a Sequence Listing which has been submitted electronically in XML format and is hereby incorporated by reference in its entirety. The XML copy, created on Sep. 11, 2025 is named 19035666.xml and is 1,991 bytes is size.

### BACKGROUND OF THE INVENTION

Cancer is a leading cause of death worldwide, accounting for nearly 10 million deaths in 2020. As the standard of care in the treatment of various cancers for several decades, chemotherapy has been successful in treating many subjects with this disease. However, chemotherapy, as it is practiced today, faces significant limitations in terms of efficacy and deleterious side effects for patients. These side effects are often the results of the inherent toxicity of chemotherapy drugs and their non-specific delivery to the site of the tumor. Most chemotherapy drugs act through anti-proliferative mechanisms or by arresting cell division at a specific phase rather than producing a toxic effect on particular types of cancer cells. These toxic chemotherapy drugs, due to the poor selectivity, affect rapidly proliferating and dividing cells such as red blood cells, hair follicles, gut epithelia, bone marrow, and the lymphatic system, as well as many other tissues in the body, making chemotherapy drugs unsuitable or intolerable for long-term treatment.

Furthermore, only 1-2% of a systemically administered chemotherapy drugs reach their desired site of action, (e.g., the cancerous tumor). Most anti-cancer drugs on the market have shown only marginal gains in terms of overall patient survival.

Moreover, chemotherapy drugs are often inefficient in treating slowly growing solid tumors, which can be problematic when addressing most human solid tumor cells, as these cells do not proliferate rapidly. As a result, high-dose chemotherapy is generally required to effectively inhibit tumor proliferation, especially in resistant solid tumors. The inefficient, non-selective nature of cancer treatment using standard chemotherapy drugs often results in lethal damage to the adjacent normal cells, which often leads to discontinuation of the therapy before all malignant cells are killed. Hence, there is an urgent need to improve therapeutic efficacy of chemotherapy treatment.

Prodrugs provide a promising approach to improve the efficacy of a chemotherapy drug. An effective prodrug can help ensure that a drug is active at a particular time and place to both maximize cancer cell elimination and minimize off-target toxicity. Instead of administering a highly toxic drug directly, a corresponding prodrug can be used to improve how the drug is absorbed, distributed, metabolized, and excreted (ADME). Provided herein, in some embodiments, are target dye-drug conjugate (DDC) prodrug compounds comprising a tumor-targeting near-infrared (NIR) dye conjugated to a chemotherapeutic agent via a labile cleavable linker system. The resulting conjugates (exemplified in FIG. 1) with cleavable linkers, are designed to be stable and safe in the circulation through the bloodstream. Once the compound reaches the targeted tumor site, the linker may break quickly in response to the unique stimulus present in the tumor environment and release the toxic payload directly to the tumor cell.

### SUMMARY OF THE INVENTION

In an aspect provided herein, the disclosure provides for a dye-drug conjugate (DDC) compound comprising:
a tumor-targeting near-infrared dye;
a chemotherapeutic drug; and
a cleavable linker,
or a pharmaceutically acceptable salt thereof.

A class of NIR fluorescent heptamethine cyanine dyes has been shown to selectively accumulate in many solid tumor types and persist in the tumor tissue from a few hours to several days. This class of NIR dyes may be used as active tumor targeting moieties and provide advantages over highly specific antibody-drug conjugates (ADC) which may accumulate in only one tumor type. These dyes may also overcome tumor heterogeneity issues faced by other active targeting approaches using for example antibodies, peptides, or aptamers. Because these dyes absorb and fluoresce in the near-infrared (NIR) region (above 750 nm), they have additional utility as optical probes for near infrared fluorescent imaging.

In some embodiments, said DDC compound is a prodrug compound. In some embodiments, the DDC compound comprises a tumor-targeting near-infrared dye. In some embodiments, said tumor-targeting near-infrared dye delivers and releases said therapeutic drug to a tumor cell.

In some embodiments, said tumor-targeting near-infrared dye releases less of said therapeutic agent to a non-tumor cell compared to a corresponding dye-drug conjugate without a cleavable linker. In some embodiments, said tumor-targeting near-infrared dye comprises a heptamethine cyanine dye.

In some embodiments, said heptamethine cyanine dye is a compound of Formula (I), or a pharmaceutically acceptable salt thereof:

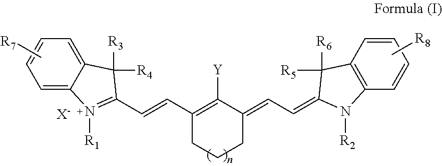

wherein:
R₁, R₂, R₃, R₄, R₅, R₆, R₇, and R₈ are each independently hydrogen, unsubstituted or substituted C₁-C₁₀alkyl, unsubstituted or substituted aryl, unsubstituted or substituted C₁-C₁₀alkoxyl, C₁-C₁₀alkylsulfonate, C₁-C₁₀alkyl carboxylic acid, or C₁-C₁₀alkylamino; wherein one or more of the alkyl, alkoxy, alkylsulfonate, alkyl carboxylic acid, or alkylamino is optionally functionalized with a zwitterion;
X is Br⁻, Cl⁻, I⁻, ClO₄⁻, or OTS⁻, or is absent if another covalently linked anion moiety is present;
Y is Cl, —C₁-C₁₀alkyl, —O—C₁-C₁₀alkyl, —S—C₁-C₁₀alkyl, or —NH—C₁-C₁₀ alkyl; and
n is 0 or 1;
wherein said linker is attached at one or more of R₁, R₂, R₃, R₄, R₅, R₆, R₇, R₈ or Y.

In some embodiments, Y is C₁-C₁₀alkyl, —O—C₁-C₁₀alkyl, —S—C₁-C₁₀alkyl, or —NH—C₁-C₁₀ alkyl. In some embodiments, Y is further substituted with a zwitterion. In some embodiments,

In some embodiments, said zwitterion is selected from sulfobetaine, phosphorylcholie, carboxybetaine, pyridinium sulfonate, imidazolium sulfonate, guanidinium and carboxylate. In some embodiments, R₃, R₄, R₅, and R₆ are each independently unsubstituted or substituted C₁-C₁₀alkyl. In some embodiments, R₃, R₄, R₅, and R₆ are each methyl. In some embodiments, R₇ and R₈ are each hydrogen. In some embodiments, R₁ and R₂ are each independently unsubstituted or substituted C₁-C₁₀alkyl, unsubstituted or substituted C₁-C₁₀alkoxyl, C₁-C₁₀alkylsulfonate, or C₁-C₁₀alkyl carboxylic acid, or C₁-C₁₀alkylamino. In some embodiments, R₁ and R₂ are each independently unsubstituted or substituted C₁-C₁₀alkyl, C₁-C₁₀alkylsulfonate, or C₁-C₁₀alkyl carboxylic acid. In some embodiments, the linker is attached at one of R₁ or R₂. In some embodiments, the linker is attached at Y. In some embodiments, n is 1.

In some embodiments, said cleavable linker comprises a protease-sensitive cleavable linker. In some embodiments, said cleavable linker comprise a Cathepsin B sensitive peptide linker. In some embodiments, said cleavable linker comprises a dipeptide. In some embodiments, said cleavable linker comprises a valine-citrulline (Val-Cit) dipeptide. In some embodiments, cleavable linker comprises a valine-alanine (Val-Ala) dipeptide. In some embodiments, cleavable linker comprises tetra-peptide sequence (Gly-Phe-Leu-Gly, GFLG). In some embodiments, said cleavable linker comprise an MMP sensitive peptide linker. In some embodiments, said cleavable linker comprise Gly-Pro-Leu-Gly-Ile-Ala-Gly-Gin (GPLGIAGQ) (SEQ ID: 1). In some embodiments, said cleavable linker comprises a pyrophosphatase, phosphatase sensitive phosphate, or pyrophosphate-containing linker. In some embodiments, said cleavable linker comprises a glycosidases sensitive glucuronic acid-containing linker. In some embodiments, said cleavable linker comprises a sulphatases sensitive sulphonate-containing linker. In some embodiments, said linker comprises polyethylene glycol. In some embodiments, said linker comprises a clickable linker. In some embodiments, said cleavable linkers further comprise a spacer. In some embodiments, said spacer is a self-immolative linker.

In some embodiments, said chemotherapeutic drug comprises an FDA approved chemotherapy. In some embodiments, said chemotherapeutic drug comprises a DNA damaging agent. In some embodiments, said chemotherapeutic drug comprises a tubulin inhibitor. In some embodiments, chemotherapeutic drug comprises clindamycin, doxorubicin, vinblastine, rifabutin, SN-38, gefitinib, MMAD, MMAF, MMAE, azonafide, indibulin, pactitax, tubulysin 5a, or a combination thereof. In some embodiments, said chemotherapeutic drug comprises doxorubicin or SN-38. In some embodiments, said chemotherapeutic drug comprises MMAE or MMAF.

In another aspect, provided herein is a composition comprising one or more compounds described herein and a pharmaceutically acceptable excipient.

In another aspect, provided herein is a method of killing a tumor cell by targeting (e.g., selectively targeting) and delivering said DDC compound to said tumor cell.

In another aspect, provided herein a method of treating cancer in a patient in need thereof, comprising administering to the patient an effective amount of a compound described herein.

Additional aspects and advantages of the present disclosure will become readily apparent to those skilled in this art from the following detailed description, wherein only illustrative embodiments of the present disclosure are shown and described. As will be understood by those skilled in the art, the present disclosure is capable of other and different embodiments, and its several details are capable of modifications in various obvious respects, all without departing from the disclosure. Accordingly, the drawings and description are illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### FIGURES

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below.

FIG. 1. shows a general design of Dye-Drug Conjugate (DDC) prodrugs with a cleavable linker.

FIG. 2. displays examples of general drug release mechanisms.

FIG. 3. displays a general drug release mechanism for prodrugs containing a PEG linker.

FIG. 4. displays a general drug release mechanism for prodrugs containing a phosphate linker.

FIG. 5. shows mean NIR intensity data over time for a lung cancer cell line (A549) and four normal cell lines (colon cells, kidney cells, liver cells, and HUVECs) following a 20 μM dose of a dye with a zwitterion moiety attached to it.

FIG. 6. shows results of a compound uptake study using NIR mean intensity (5 μM).

FIG. 7. shows results of a compound uptake study using nuclei counts (5 μM).

FIG. 8. shows the viability of A549 cells 24 h after the cells were dosed with Dye 1 alone, Dye 2 alone, Dye 1-SN38, Dye 1-Doxorubicin (“Doxo”), Dye 2-SN38, and Dye 2-Doxo.

FIG. 9. shows the viability of A549 2D Cell Viability 48 h after the cells were dosed with Dye 1 alone, Dye 2 alone, Dye 1-SN38, Dye 1-Doxorubicin (“Doxo”), Dye 2-SN38, and Dye 2-Doxo.

FIGS. 10A and 10B. show SW620 tumor bearing mice with intravenous tail injection with Compound XV (NIR Dye-SN38) 11 days after the second dosing at 15 mg/kg, 10 sec exposure. 10A shows the tumor side and 10B shows the dorsal site.

FIGS. 11A and 11B. show SW620 tumor bearing mice with intravenous tail injection with Compound XVI (NIR Dye-Doxo) 11 days after the second dosing at 15 mg/kg, 10 sec exposure. 11A shows the tumor side and 11B shows the dorsal site.

FIGS. 12A and 12B. show organ uptake in a mouse bearing SW620 tumor as judged by NIR fluorescent imaging. 12A shows the uptake with vehicle (50% DMSO: 40% PEG400: 10% ethanol) and 12B shows the organ uptake with Compound XV after a second dose of 15 mg/kg after 24 h injection-auto exposure.

FIGS. 13A and 13B. show organ NIR fluorescent imaging of a mouse bearing SW620 tumor with Compound XVI. 13A shows organ imaging after a first dose (10 mg/kg, day 17 after the injection, 10 sec exposure) and 13B shows organ imaging after a second dose (15 mg/kg, day 7 after the injection, 10 sec exposure).

FIGS. 14A and 14B. show organ NIR fluorescent imaging of a mouse bearing SW620 tumor with control NIR dye-1. 14A shows imaging after a first dose at 10 mg/kg day 17 after the injection (10 sec exposure) and 14B shows imaging after a second dose at 4.37 mg/kg day 7 after the injection (10 sec exposure).

FIG. 15. shows tumor size changes with Dye 1-SN38 (XV) with 10 mg/kg first dose and a second dose 15 mg/kg on Day 11 via intravenous tail injection.

FIG. 16. shows tumor size (mm³) changes with Dye 1-SN38 (XV) via intratumor injection at 15 mg/kg dose.

FIG. 17. tumor size (mm³) changes of Dye 1-Doxo (XVI) via intratumor injection at 15 mg/kg dose.

FIG. 18. shows the LCMS data for compound Dye2-triazole-PEG8-Val-Cit-PABC-Doxorubicin (compound XVIII).

FIG. 19. shows the 1H NMR data for compound Dye2-triazole-PEG8-Val-Cit-PABC-Doxorubicin (compound XVIII).

FIG. 20. shows the LCMS data for compound Dye2-triazole-PEG8-Val-Cit-PABC-SN-38 (compound XVII).

FIG. 21. shows the 1H NMR data for compound Dye2-triazole-PEG8-Val-Cit-PABC-SN-38 (compound XVII).

FIG. 22. shows the LCMS data for compound Dye1-PEG8-Val-Cit-PABA-SN-38 (compound XV).

FIG. 23. shows the 1H NMR data for compound Dye1-PEG8-Val-Cit-PABA-SN-38 (compound XV).

FIG. 24. shows the LCMS data for compound Dye1-PEG8-Val-Cit-PABA-Doxorubicin (compound XVI).

FIG. 25. shows the 1H NMR data for compound Dye1-PEG8-Val-Cit-PABA-Doxorubicin (compound XVI).

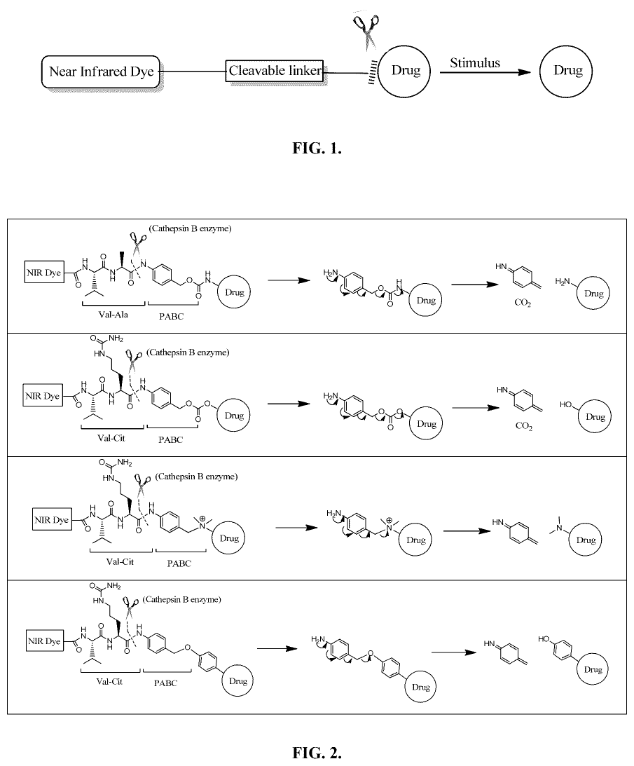

### DETAILED DESCRIPTION OF THE INVENTION

### Compounds

In some aspects, the disclosure provides for a dye-drug conjugate (DDC) compound. In some embodiments, said DDC compound is a prodrug.

In some embodiments, the disclosure provides a dye-drug conjugate compound comprising:
a tumor-targeting near-infrared dye;
a chemotherapeutic drug; and
a cleavable linker,
or a pharmaceutically acceptable salt thereof.

In some embodiments, said tumor-targeting near-infrared dye delivers and releases said therapeutic drug to a tumor cell.

### Tumor-Targeting Near-Infrared (NIR) Dye

The tumor targeting NIR dye may serve a dual function for delivering and imaging of the prodrug, which contrasts with the non-specific delivery of conventional anti-cancer drugs, which can cause significant, adverse side effects. In addition to targeting tumor cells, the attached dye also provides NIR imaging capability such that the prodrug can be detected throughout the drug delivery process. Such a property is desirable considering the non-invasive nature of NIR light and its relatively deep tissue penetration, which is orders of magnitude greater than that for ultraviolet or visible light. Thus, the DDC compound may provide both a tumor targeted drug delivery system and a theranostic prodrug that is equipped with fluorophores as optical reporters that enable real-time monitoring of the drug delivery and release process. In some embodiments, said DDC further comprises an optical reporter. In some embodiments, said optical reporter comprises a fluorophore.

In some embodiments, said tumor-targeting near-infrared dye comprises a heptamethine cyanine dye.

In some embodiment, said heptamethine cyanine dye is a compound of Formula (I), or a pharmaceutically acceptable salt thereof:

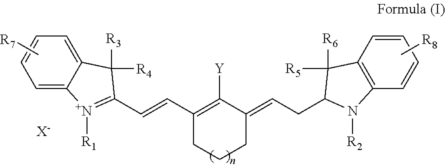

wherein:
R₁, R₂, R₃, R₄, R₅, R₆, R₇, and R₈ are each independently hydrogen, unsubstituted or substituted C₁-C₁₀alkyl, unsubstituted or substituted aryl, unsubstituted or substituted C₁-C₁₀alkoxyl, C₁-C₁₀alkylsulfonate, C₁-C₁₀alkyl carboxylic acid, or C₁-C₁₀alkylamino; wherein one or more of the alkyl, alkoxy, alkylsulfonate, alkyl carboxylic acid, or alkylamino is optionally functionalized with a zwitterion;
X is Br⁻, Cl⁻, I⁻, ClO₄⁻, or OTS⁻, or is absent if another covalently linked anion moiety is present;
Y is Cl, —C₁-C₁₀alkyl, —O—C₁-C₁₀alkyl, —S—C₁-C₁₀alkyl, or —NH—C₁-C₁₀ alkyl; and
n is 0 or 1;
   wherein said linker is attached at one or more of R₁, R₂, R₃, R₄, R₅, R₆, R₇, R₈ or Y.

In some embodiments of Formula (I), said dry-drug conjugate compound comprises:
R₁ and R₂ are each independently unsubstituted or substituted C₁-C₁₀alkyl, unsubstituted or substituted aryl, unsubstituted or substituted C₁-C₁₀alkoxyl, C₁-C₁₀alkylsulfonate, C₁-C₁₀alkyl carboxylic acid, or C₁-C₁₀alkylamino; wherein one or more of the alkyl, alkoxy, alkylsulfonate, alkyl carboxylic acid, or alkylamino is optionally functionalized with a zwitterion;
R₃, R₄, R₅, and R₆ are each independently unsubstituted or substituted C₁-C₁₀alkyl;
R₇, and R₈ are each hydrogen;
X is Br⁻, Cl⁻, I⁻, ClO₄⁻, or OTS⁻, or is absent if another covalently linked anion moiety is present;
Y is —O—C₁-C₁₀alkyl, —S—C₁-C₁₀alkyl, or —NH—C₁-C₁₀ alkyl; and n is 1;
   wherein said linker is attached at one of R₁, R₂, or Y.

In some embodiments, R₁, R₂, R₃, R₄, R₅, R₆, R₇, and R₈ are each independently hydrogen, unsubstituted or substituted C₁-C₁₀alkyl, unsubstituted or substituted C₁-C₁₀alkoxyl, C₁-C₁₀alkylsulfonate, C₁-C₁₀alkyl carboxylic acid, or C₁-C₁₀alkylamino; wherein one or more of the alkyl, alkoxy, alkylsulfonate, alkyl carboxylic acid, or alkylamino is optionally functionalized with a zwitterion.

In some embodiments, R₁ and R₂ are each independently unsubstituted or substituted C₁-C₁₀alkyl, unsubstituted or substituted C₁-C₁₀alkoxyl, C₁-C₁₀alkylsulfonate, or C₁-C₁₀alkyl carboxylic acid, or C₁-C₁₀alkylamino. In some embodiments, R₁ and R₂ are each independently unsubstituted or substituted C₁-C₁₀alkyl, C₁-C₁₀alkylsulfonate, or C₁-C₁₀alkyl carboxylic acid. In some embodiments, R₁ and R₂ are each independently unsubstituted or substituted C₁-C₁₀alkyl. In some embodiments, R₁ and R₂ are each independently C₁-C₁₀alkylsulfonate. In some embodiments, R₁ and R₂ are each independently C₁-C₁₀alkyl carboxylic acid. In some embodiments, one or two of R₁ and R₂ is optionally functionalized with a zwitterion.

In some embodiments, R₃, R₄, R₅, and R₆ are each independently unsubstituted or substituted C₁-C₁₀alkyl. In some embodiments, R₃, R₄, R₅, and R₆ are each independently methyl, ethyl, or propyl. In some embodiments, R₃, R₄, R₅, and R₆ are each methyl. In some embodiments, R₃, R₄, R₅, and R₆ are each ethyl. In some embodiments, R₃, R₄, R₅, and R₆ are each propyl.

In some embodiments, R₇ and R₈ are each hydrogen.

In some embodiments, Y is —C₁-C₁₀alkyl, —O—C₁-C₁₀alkyl, —S—C₁-C₁₀alkyl, or —NH—C₁-C₁₀ alkyl. In some embodiments, Y is —C₁-C₁₀alkyl. In some embodiments, Y is —O—C₁-C₁₀alkyl, —S—C₁-C₁₀alkyl, or —NH—C₁-C₁₀ alkyl. In some embodiments, Y is —O—C₁-C₁₀alkyl or —S—C₁-C₁₀alkyl. In some embodiments, Y is —O—C₁-C₁₀alkyl. In some embodiments, Y is —S—C₁-C₁₀alkyl. In some embodiments, Y is —NH—C₁-C₁₀ alkyl. In some embodiments, Y is Cl.

In some embodiments, Y is further substituted with a zwitterion.

The presence of zwitterionic functionality serves to increase aqueous solubility, bio-compatibility, and tumor uptake selectivity of the resulting prodrug compounds.

The highly hydrated zwitterions lead to a tightly bound water layer as a dense and stable hydration shell or solvation shell which provides a physical and energetic barrier that can prevent undesired adsorption, such as but not limited to, protein adsorption; thus, zwitterions have effective anti-fouling properties in this context.

In addition, zwitterionic molecules exhibit very low levels of non-specific protein binding upon exposure to serum, and negligible uptake by phagocytic and non-phagocytic hepatocarcinoma cells, which suggests that zwitterions are able to resist rapid accumulation in the liver and spleen, a common in vivo fate for many drug molecules.

In some embodiments, said zwitterion is selected from sulfobetaine, phosphorylcholine, carboxybetaine, pyridinium sulfonate, imidazolium sulfonate, guanidinium, and carboxylate. In some embodiments, said zwitterion is sulfobetaine. In some embodiments, said zwitterion is phosphorylcholine. In some embodiments, said zwitterion is carboxybetaine. In some embodiments, said zwitterion is pyridinium sulfonate. In some embodiments, said zwitterion is imidazolium sulfonate. In some embodiments, said zwitterion is guanidinium. In some embodiments, said zwitterion is carboxylate.

In some embodiments, said zwitterion is selected from Table 1.

TABLE 1
Exemplary zwitterions.

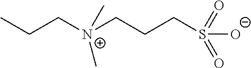

Sulfobetaine

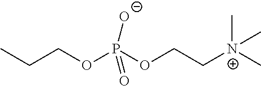

phosphorylcholine

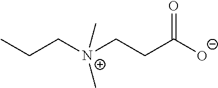

carboxybetaine

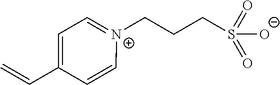

Pyridinium sulfonate

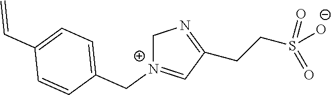 [SMILES: C=Cc1ccc(C[N+]2=CC(CCS(=O)(=O)[O-])=NC2)cc1]

Imidazolium sulfonate

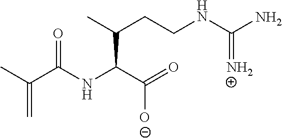 [SMILES: C=C(C)C(=O)N[C@H](C(=O)[O-])C(C)CCNC(N)=[NH2+]]

Guanidinium carboxylate

In some embodiments, n is 1. In some embodiments, n is 0.

In some embodiments, said linker is attached at one, two, three or four of R₁, R₂, R₃, R₄, R₅, R₆, R₇, R₈ or Y. In some embodiments, said linker is attached at one, two, or three of R₁, R₂, R₃, R₄, R₅, R₆, R₇, R₈ or Y. In some embodiments, said linker is attached at one or two of R₁, R₂, R₃, R₄, R₅, R₆, R₇, R₈ or Y. In some embodiments, said linker is attached at one or more of R₁, R₂, or Y. In some embodiments, said linker is attached at Y. In some embodiments, said linker is attached at one or both of R₁ or R₂. In some embodiments, said linker is attached at one of R₁ or R₂. In some embodiments, said linker is attached at R₁. In some embodiments, said linker is attached at R₂.

In some embodiments, X is Br⁻, Cl⁻, I⁻, ClO₄⁻, or OTS⁻. In some embodiments, X is Br⁻, Cl⁻, or I⁻. In some embodiments, X is Br⁻. In some embodiments, X is Cl⁻. In some embodiments, X is I⁻. In some embodiments, X is ClO₄⁻ or OTS⁻. In some embodiments, X is ClO₄⁻. In some embodiments, X is OTS⁻. In some embodiments, X is absent if another covalently linked anion moiety is present.

In some embodiments, said tumor-targeting NIR dye of Formula (I), is selected from Table 2.

TABLE 2
Tumor targeting NIR dyes.

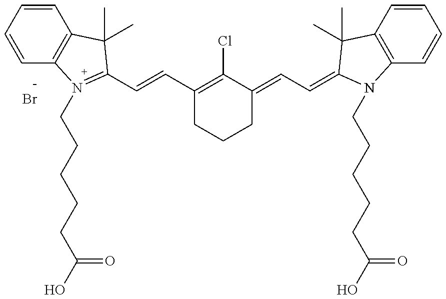

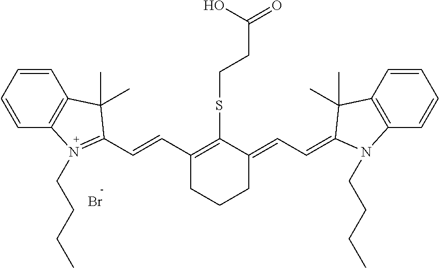

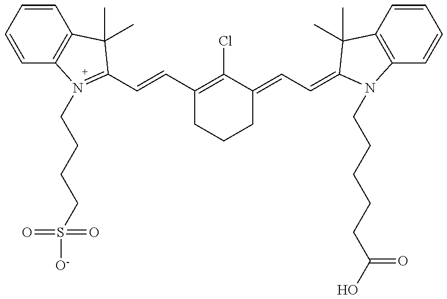

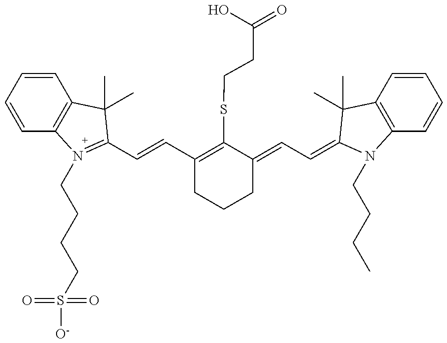

“Dye 3”

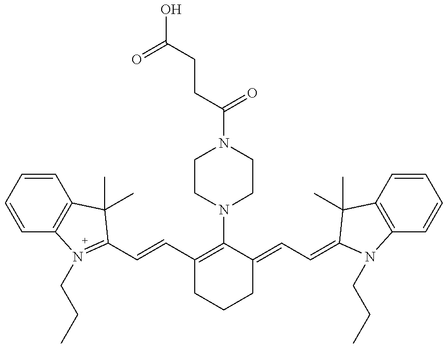

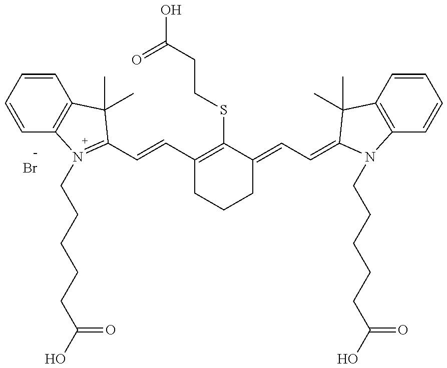

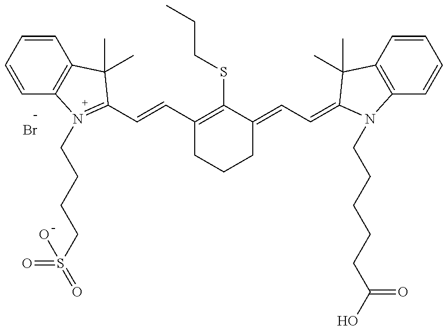

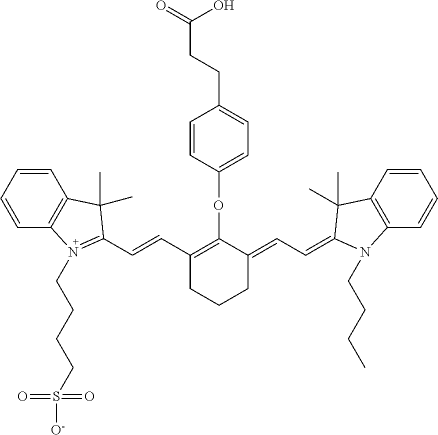

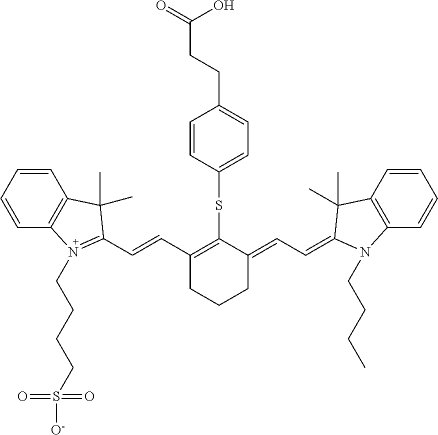

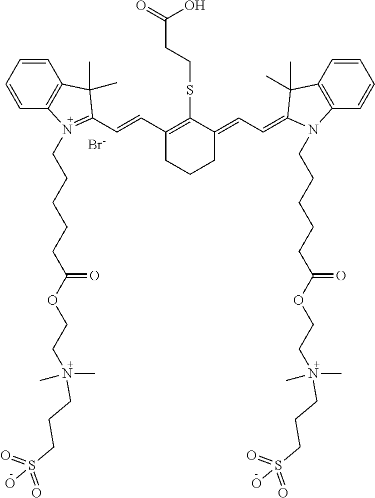

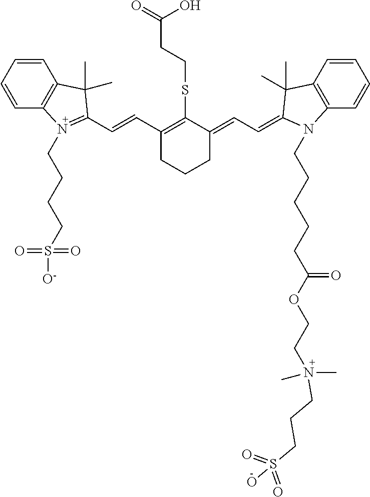

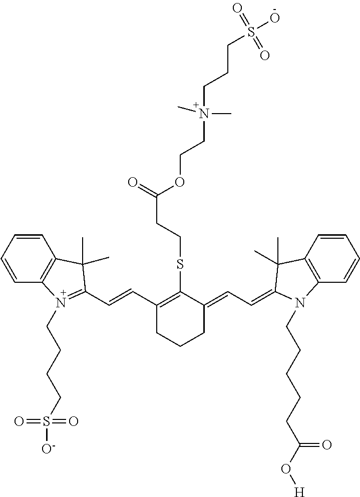

### Chemotherapeutic Drugs

Another component of said DDC compound is a cytotoxin (e.g., a chemotherapeutic drug, also known as the “payload”). The payload determines the ability of the resulting DDC to kill cancer cells. The basic parameters for selecting an effective payload for the DDC include, but are not limited to, its toxicity, stability, solubility, and its chemical functionality so that it can be coupled to a linker and a NIR dye of Formula (I).

According to their mechanism of action, the cytotoxin of the DDC compound can be divided into two categories: DNA damaging agents and tubulin inhibitors. The action of DNA-damaging agents is independent of the cell growth process. They are often described as molecular scissors because of their ability to cleave genomic DNA; thereby, leading to cell death. Doxorubicin and SN-38 belong to this class of compounds. Tubulin inhibitors act during cell growth through mitotic arrest leading to cell death. Compounds of this class are monomethyl auristatin E (MMAE) and F (MMAF).

In some embodiments, the chemotherapeutic drug comprises an FDA approved chemotherapy.

In some embodiments, said chemotherapeutic drug comprises a DNA damaging agent.

In some embodiments, said chemotherapeutic drug is a tubulin inhibitor.

In some embodiments, said chemotherapeutic drug comprises clindamycin, doxorubicin, vinblastine, rifabutin, SN-38, gefitinib, MMAD, MMAF, MMAE, azonafide, indibulin, pactitax, tubulysin 5a, or any combination thereof. In some embodiments, said chemotherapeutic drug comprises doxorubicin or SN-38. In some embodiments, said chemotherapeutic agent comprises an antimitotic agent. In some embodiments, said chemotherapeutic drug comprises a camptothecin analog. In some embodiments, said chemotherapeutic drug comprises a topoisomerase I inhibitor. In some embodiments, said chemotherapeutic drug comprises SN-38. In some embodiments, said chemotherapeutic drug comprises an anthracycline. In some embodiments, said chemotherapeutic drug comprises doxorubicin. In some embodiments, said chemotherapeutic agent comprises a tubulin inhibitor. In some embodiments, said chemotherapeutic drug comprises MMAE or MMAF. In some embodiments, said chemotherapeutic drug comprises MMAE. In some embodiments, said chemotherapeutic drug comprises MMAF. In some embodiments, said chemotherapeutic drug comprises MMAD. In some embodiments, said chemotherapeutic drug comprises an antibiotic. In some embodiment, said chemotherapeutic agent comprises a synthetic antineoplastic agent. In some embodiments, said chemotherapeutic drug comprises clindamycin. In some embodiment, said chemotherapeutic agent comprises a vinca alkaloid. In some embodiments, said chemotherapeutic drug comprises vinblastine. In some embodiments, said chemotherapeutic drug comprises rifabutin. In some embodiment, said chemotherapeutic agent comprises an epidermal growth factor receptor (EGFR) inhibitor. In some embodiments, said chemotherapeutic drug comprises gefitinib. In some embodiment, said chemotherapeutic agent comprises a naphthalimide. In some embodiments, said chemotherapeutic drug comprises azonafide. In some embodiment, said chemotherapeutic agent comprises a n-alkylindole. In some embodiments, said chemotherapeutic drug comprises indibulin. In some embodiment, said chemotherapeutic agent comprises a taxane. In some embodiments, said chemotherapeutic drug comprises pactitax. In some embodiments, said chemotherapeutic drug comprises a tubulysin. In some embodiments, said chemotherapeutic drug comprises tubulysin 5a.

In some embodiments, said chemotherapeutic drug comprises a chemotherapeutic drug selected from Table 3.

TABLE 3
Chemotherapy drugs that can be used in DDC.

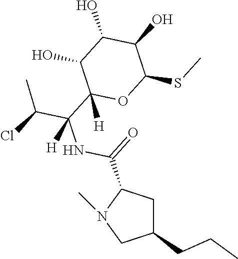 [SMILES: CCC[C@@H]1C[C@@H](C(=O)N[C@@H]([C@H]2O[C@H](SC)[C@H](O)[C@@H](O)[C@H]2O)[C@H](C)Cl)N(C)C1]

Clindamycin

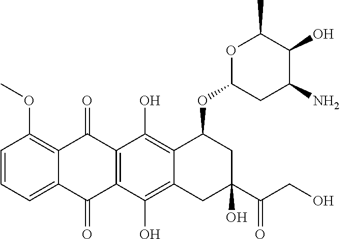 [SMILES: COc1cccc2c1C(=O)c1c(O)c3c(c(O)c1C2=O)C[C@@](O)(C(=O)CO)C[C@@H]3O[C@H]1C[C@H](N)[C@H](O)[C@H](C)O1]

Doxorubicin

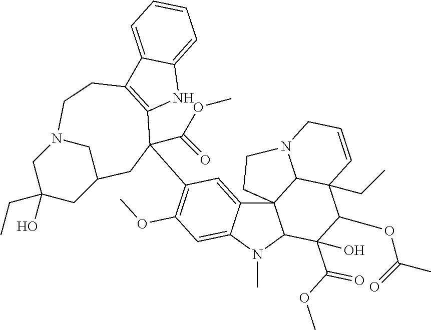 [SMILES: CCC1(O)CC2CN(CCc3c([nH]c4ccccc34)C(C(=O)OC)(c3cc4c(cc3OC)N(C)C3C(O)(C(=O)OC)C(OC(C)=O)C5(CC)C=CCN6CCC43C65)C2)C1]

vinblastine

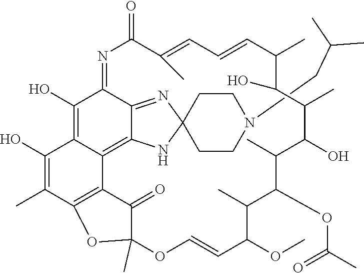 [SMILES: COC1/C=C/OC2(C)Oc3c(C)c(O)c4c(O)/c(c5c(c4c3C2=O)NC2(CCN(CC(C)C)CC2)N=5)=N/C(=O)/C(C)=C/C=C/C(C)C(O)C(C)C(O)C(C)C(OC(C)=O)C1C]

rifabutin

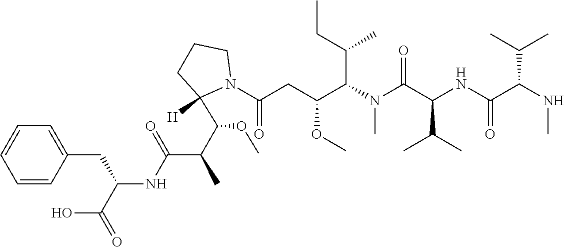 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

MMAE

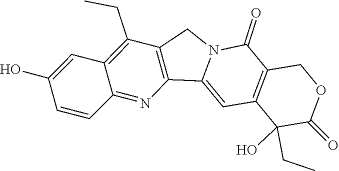 [SMILES: CCc1c2c(nc3ccc(O)cc13)-c1cc3c(c(=O)n1C2)COC(=O)C3(O)CC]

SN-38

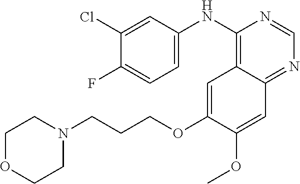 [SMILES: COc1cc2ncnc(Nc3ccc(F)c(Cl)c3)c2cc1OCCCN1CCOCC1]

Gefitinib

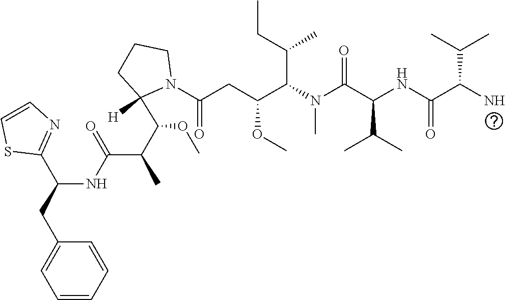 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@@H](N)C(C)C)C(C)C]

MMAD

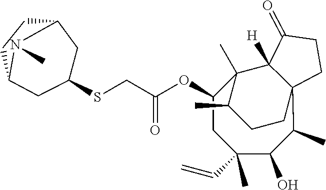

Retapamulin

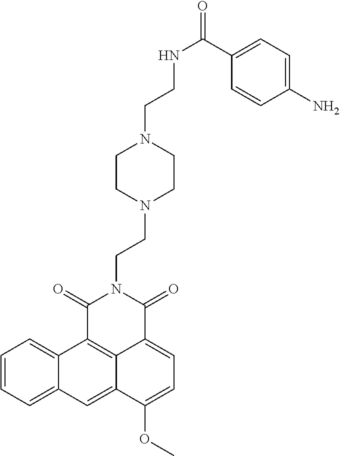 [SMILES: COc1ccc2c3c(c4ccccc4cc13)C(=O)N(CCN1CCN(CCNC(=O)c3ccc(N)cc3)CC1)C2=O]

Azonafide

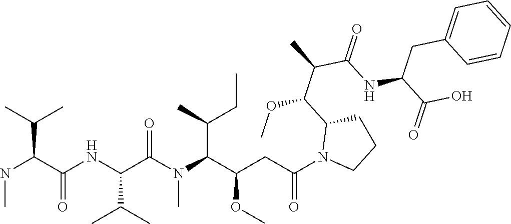 [SMILES: CC[C@H](C)[C@@H]([C@@H](CC(=O)N1CCC[C@H]1[C@H](OC)[C@@H](C)C(=O)N[C@@H](Cc1ccccc1)C(=O)O)OC)N(C)C(=O)[C@@H](NC(=O)[C@@H](NC)C(C)C)C(C)C]

MMAF

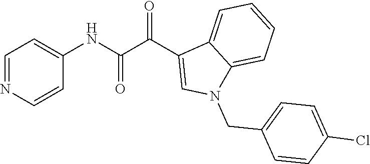 [SMILES: O=C(Nc1ccncc1)C(=O)c1cn(Cc2ccc(Cl)cc2)c2ccccc12]

Indibulin

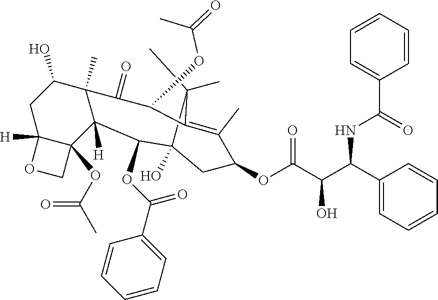 [SMILES: CC(=O)O[C@H]1C(=O)[C@@]2(C)[C@H]([C@H](OC(=O)c3ccccc3)[C@]3(O)C[C@H](OC(=O)[C@H](O)[C@@H](NC(=O)c4ccccc4)c4ccccc4)C(C)=C1C3(C)C)[C@]1(OC(C)=O)CO[C@@H]1C[C@@H]2O]

Paclitaxel

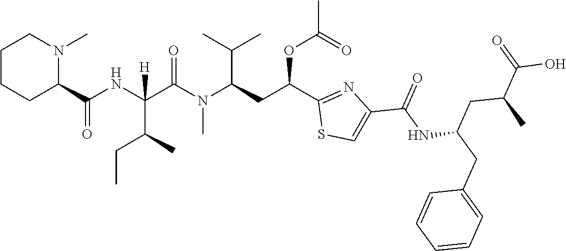 [SMILES: CC[C@H](C)[C@H](NC(=O)[C@H]1CCCCN1C)C(=O)N(C)[C@H](C[C@@H](OC(C)=O)c1nc(C(=O)N[C@@H](Cc2ccccc2)C[C@H](C)C(=O)O)cs1)C(C)C]

Tubulysin 5a

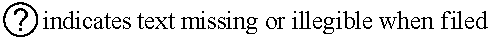

### Cleavable Linker

In some embodiments, said DDC compound comprises a cleavable linker connecting said tumor-targeting near-infrared dye of Formula (I) and said chemotherapeutic drug.

The chemical nature of said linker may play a crucial role in the stability, pharmacokinetic, and pharmacodynamic properties of the resulting DDC compound. The ideal linker should provide stable conjugates to prevent premature release of the payload (e.g., the chemotherapeutic drug) into the plasma, as this may result in undesired effects. The conjugated payload can be effectively released once it reaches the targeted site with the unique stimulus only present in a tumor environment.

According to different cleaving mechanisms, cleavable linkers can be divided into chemically cleavable linkers and enzymatically cleavable linkers. Exemplary chemically cleavable linkers include primarily acid-labile linkers including hydrazones, carbonates, and silyl ethers. For acid-labile linkers, intracellular release of the payload relies on the different pH between endosomes/lysosomes and the blood. For example, a lower pH of the endosome (e.g., pH=5-6) and lysosome (e.g., pH=4.8) compartments compared to the cytoplasm (e.g., pH=7.4) can trigger the hydrolysis of an acid-labile group within the linker, which is otherwise relatively stable at neutral pH, but unstable at pH 5. In some embodiments, said linker is an acid labile linker. In some embodiments, said linker cleaves at a pH below about 5. In some embodiments, said linker cleaves at a pH below about 5.

Enzymatically cleavable linkers, such as peptide linkers, are stable in the blood stream but can be readily cleaved in intracellular compartments by specific tumor-associated proteases. The peptide linkers are stable in the systemic circulation due to the presence of protease inhibitors in the blood.

Cathepsin B is a lysosomal protease, which is often over-expressed in various cancer cells and participates in many carcinogenic processes in humans. Cathepsin B has a relatively wide substrate range, but preferentially recognizes certain sequences, such as phenylalanine-lysine (Phe-Lys), valine-alanine (Val-Ala), and valine-citrulline (Val-Cit). The C-terminus of such peptide sequences gets cleaved by the enzyme. An advantage of these linkers is that they maintain the prodrug stability in blood circulation and then enable the rapid release of cytotoxic drugs in the tumor site.

In some embodiments, said linker is an enzyme cleavable linker. In some embodiments, said cleavable linker comprises a protease-sensitive cleavable linker. In some embodiments, said linker comprises a Cathepsin B cleavable valine-citrulline (Val-Cit) dipeptide. In some embodiments, said cleavable linker comprises valine-alanine (Val-Ala). In some embodiments, cleavable linker comprises tetra-peptide sequence (Gly-Phe-Leu-Gly, GFLG).

Matrix metalloproteinases (MMPs) are known to be involved and overexpressed in many stages of human cancers. Synthetic octapeptide, Gly-Pro-Leu-Gly-Ile-Ala-Gly-Gln (GPLGIAGQ) (SEQ ID NO: 1), is specific to the MMP2 enzyme. In some embodiments, said linker comprises a MMP sensitive peptide linker. In some embodiments, said linker comprises GPLGIAGQ (SEQ ID NO: 1).

Examples of enzyme cleavable linkers include, but are not limited to, the following linkers shown below in Table 4.

TABLE 4
Examplery enzyme cleavable linkers.

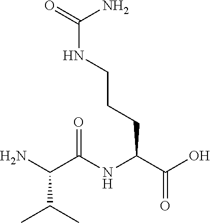 [SMILES: CC(C)[C@H](N)C(=O)N[C@@H](CCCNC(N)=O)C(=O)O]

Val-Cit

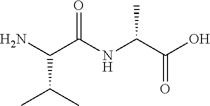

Val-Ala

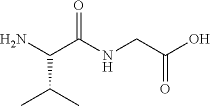

Val-Gly

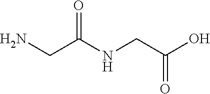

Gly-Gly

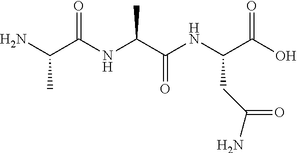 [SMILES: C[C@H](N)C(=O)N[C@@H](C)C(=O)N[C@@H](CC(N)=O)C(=O)O]

Ala-Ala-Asn

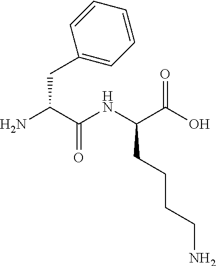 [SMILES: NCCCC[C@@H](NC(=O)[C@H](N)Cc1ccccc1)C(=O)O]

Phe-Lys

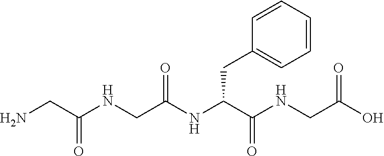 [SMILES: NCC(=O)NCC(=O)N[C@H](Cc1ccccc1)C(=O)NCC(=O)O]

Gly-Gly-Phe-Gly

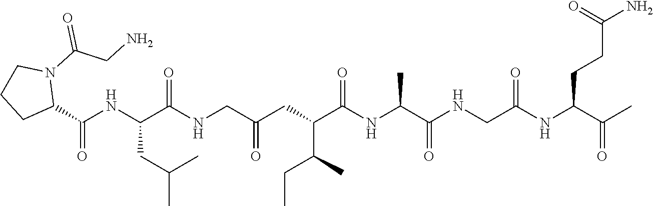

GPLGIAGQ, MMP2-cleavable polypeptide

Because of the size of the enzyme Cathepsin B and its space demand, a spacer may need to be inserted between said peptide linker and said drug. The spacer serves a dual function of relieving the steric hindrance and facilitating enzyme access. More importantly, it may act as a self-immolative moiety to release the intact chemotherapy drugs once cleaved in the presence of an enzyme and/or a change in pH.

As to particular embodiments, the linker comprises two parts: (i) a first part that links the tumor-targeting NIR dye and the protease-sensitive cleavable peptide, and (ii) a second part that links the protease-sensitive, cleavable peptide and the chemotherapeutic drug. The first part is stable in circulation and does not need to be cleavable, whereas the second part is stable in circulation and is readily cleavable, once the conjugate is delivered to the tumor site, such that the chemotherapeutic agent is released and/or pharmacologically activated. Correspondingly, a self-immolative spacer may be inserted between the protease-sensitive cleavable peptide and the chemotherapeutic agent to facilitate effective release of the agent.

In some embodiments, said cleavable linker further comprises a spacer. In some embodiments, said spacer is a self-immolative linker.

Self-immolative moieties include, but are not limited, to para-amino-benzyl carbamate (PABC). Once the peptide bond is cleaved by the enzyme at the tumor site, the spacer may undergo spontaneous 1,6-elimination in acidic media, releasing carbon dioxide, para aza-quinone methide and the chemotherapy drug in its original toxic form for cancer cell destruction. Thereby, these moieties may enable the free payload molecules to release in a traceless manner. Examples of drug release mechanisms are shown in FIG. 2. Without being bound by theory, the drug release process starts with the cleavage of a Cathepsin B sensitive peptide, which triggers the self-immolative linker PABC to undergo a 1,6-elimination. As a result, the free drug, in its parent hydroxyl, aminal, or disubstituted aminnal forms, may be released together with carbon dioxide, and para aza-quinone methide as byproducts.

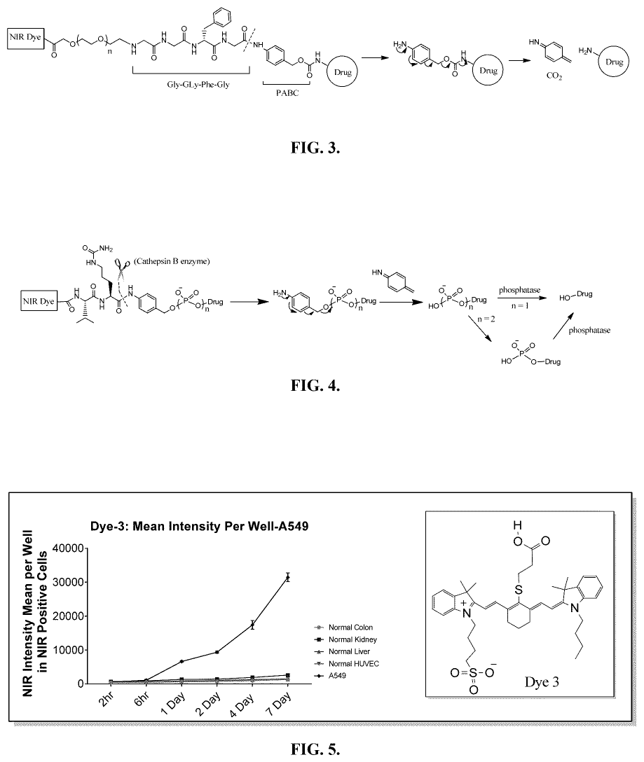

Due to the hydrophobic nature of the tumor-targeting NIR dye, chemotherapy drugs, and some peptides, an additional polyetheyle glycol (PEG) moiety with appropriate length, is often needed as a part of the linker, in order to achieve the required solubility of the final DDC compound. The PEG moiety is inserted between the dye and the peptide. Its presence does not alter the drug releasing mechanism. FIG. 3 shows the general drug release mechanism involving a prodrug containing a PEG group. In some embodiments, said linker includes polyethylene glycol (PEG). In some embodiments, said PEG increases solubility and provides the desired drug metabolism and pharmacokinetics (DMPK) of the delivered chemotherapeutic agent.

Similar to Cathepsin, pyrophosphatase and phosphatase are hydrolases selectively expressed in lysosomes, phosphate and pyrophosphate-containing linkers can be rapidly cleaved by these enzymes. When these linkers are coupled with a Cathepsin B sensitive Val-Cit-PABA moiety, together with a tumor targeting dye moiety, the resulting conjugates can deliver the desired payload (FIG. 4). Without being bound by theory, the payload is released in the following order: Cathepsin B, self-degradation spacer and then phosphatase (n=1). For pyrophosphate (n=2), another step involving pyrophosphatase may be required. In addition, the hydrophilic and anionic-charged group, present in the phosphate or pyrophosphate, possesses higher water solubility than traditional linkers and excellent cycle stability.

β-Glucuronidases are glycosidases that are highly expressed in lysosomes and tumor stroma. These enzymes catalyze β-hydrolysis of glucuronic acid residues. When the β-glucuronic moiety is coupled with a self-eliminating spacer, the resulting DDC compound can be used to deliver the desired payload through the cleavable linker together with the self-degradation spacer. This glucuronic acid-containing linker can also be applied to other hydroxyl containing payloads such as, for example, camptothecin analogues, SN-38, duramycin and matrine through an additional dimethylethylenediamine (DMED) self-degradation spacer. The glucuronic acid containing cleavable linker offers higher water solubility.

Sulphatases are also a cleavable enzyme for sulphonate groups. In addition, the negatively charged sulphonate group helps to increase the hydrophilicity of the DDC compound, which is of particular importance, as the payload is often hydrophobic.

In some embodiments, said cleavable linker comprises a pyrophosphatase, phosphatase sensitive phosphate, or pyrophosphate-containing linker. In some embodiments, said cleavable linker comprises a glycosidases sensitive glucuronic acid-containing linker. In some embodiments, said cleavable linker comprises a sulphatases sensitive sulphonate-containing linker.

Click chemistry can be used to link said tumor targeting NIR dye and the cleavable peptide sequence. In some embodiments, said linker comprises a clickable linker. As one illustrative example, dibenzocyclooctyne (DBCO) reagents include a highly reactive DBCO group which can react with azide-tagged molecules or biomolecules via copper-free click chemistry. DBCO click chemistry can be run in an aqueous buffer or in organic solvents depending on the property of the substrate molecules.

Any combination of the groups described above for the various variables is contemplated herein. Throughout the specification, groups and substituents thereof are chosen by one skilled in the field to provide stable moieties and compounds.

In some embodiments, the dye-drug conjugate compound is selected from:

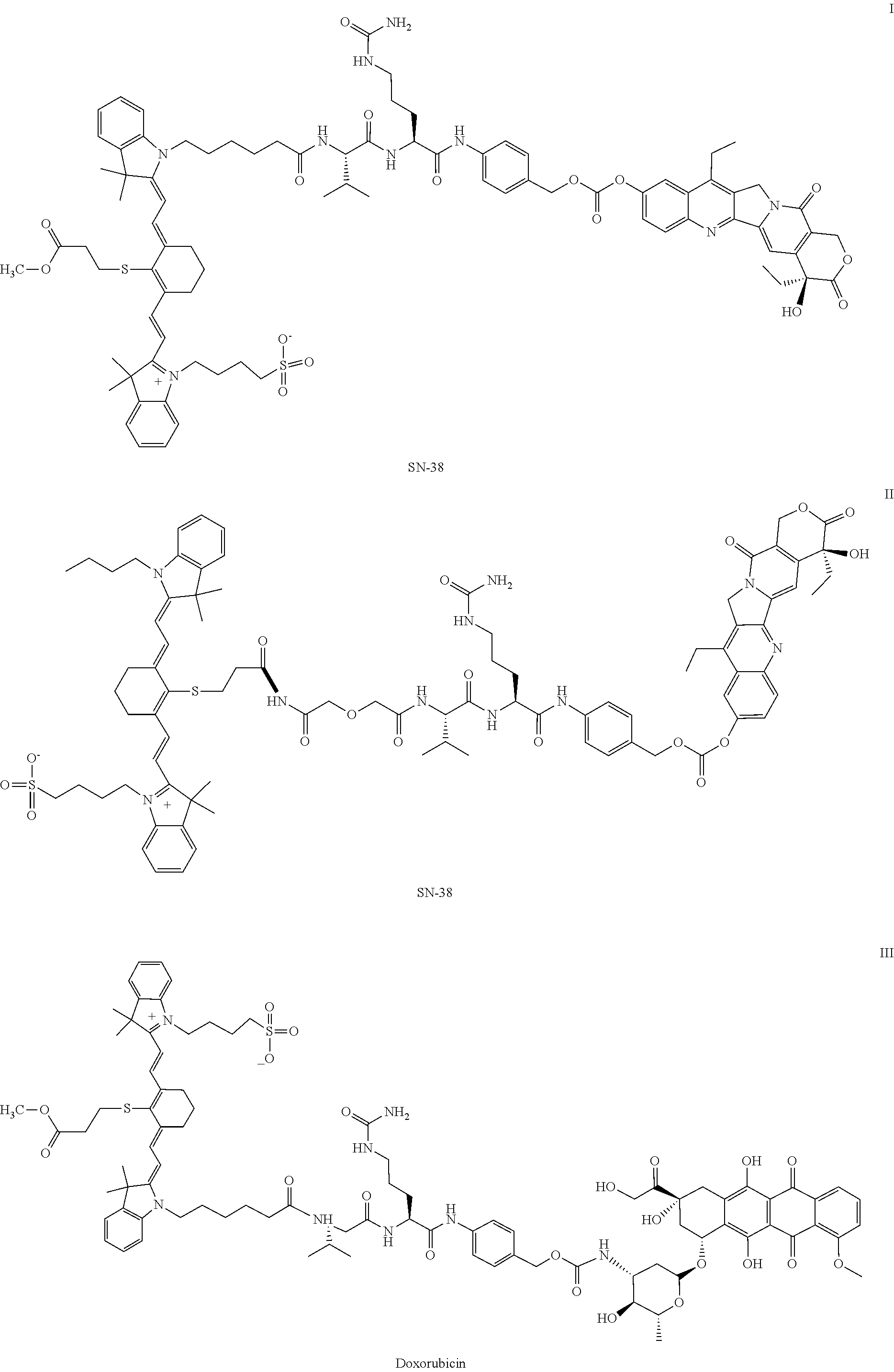

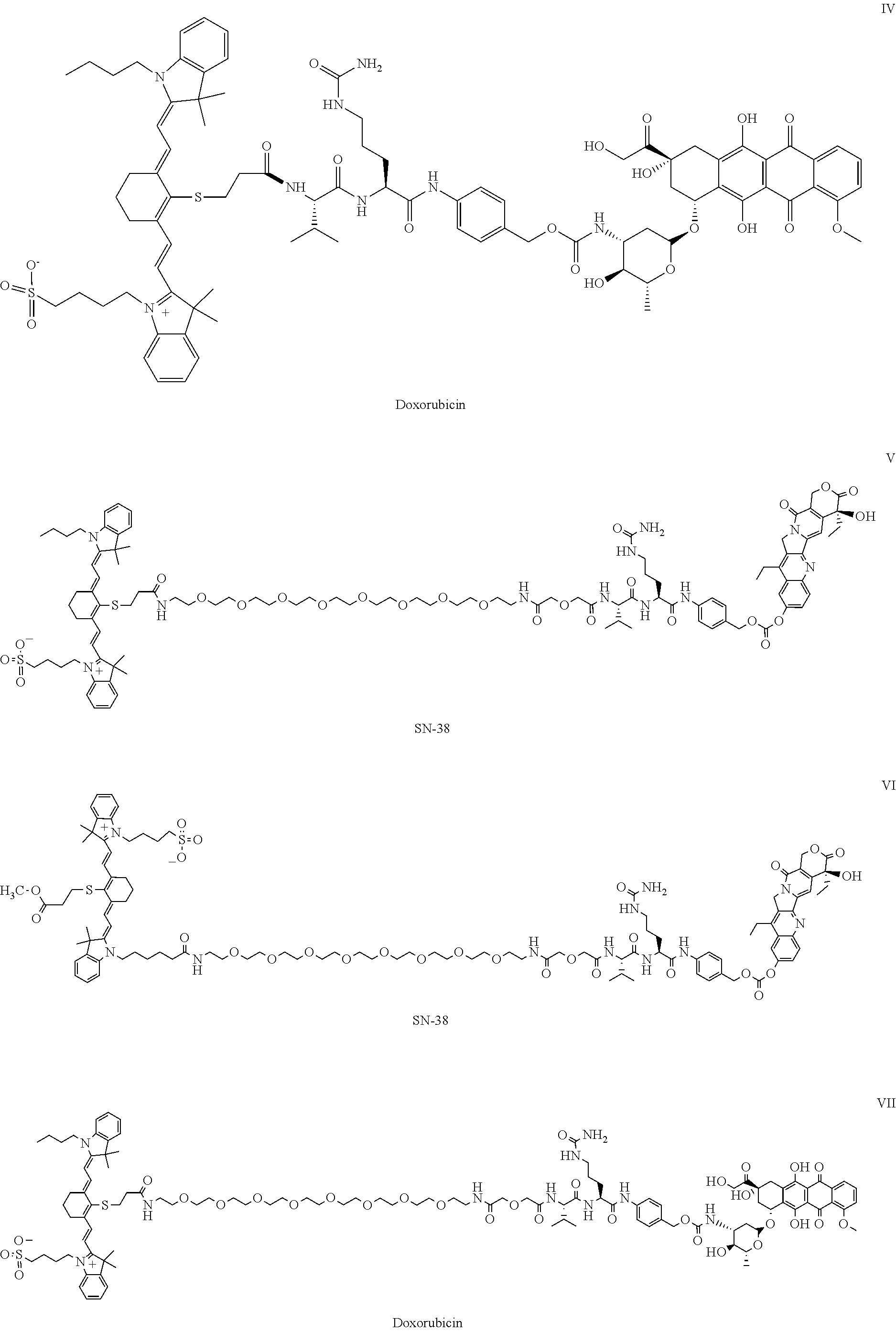

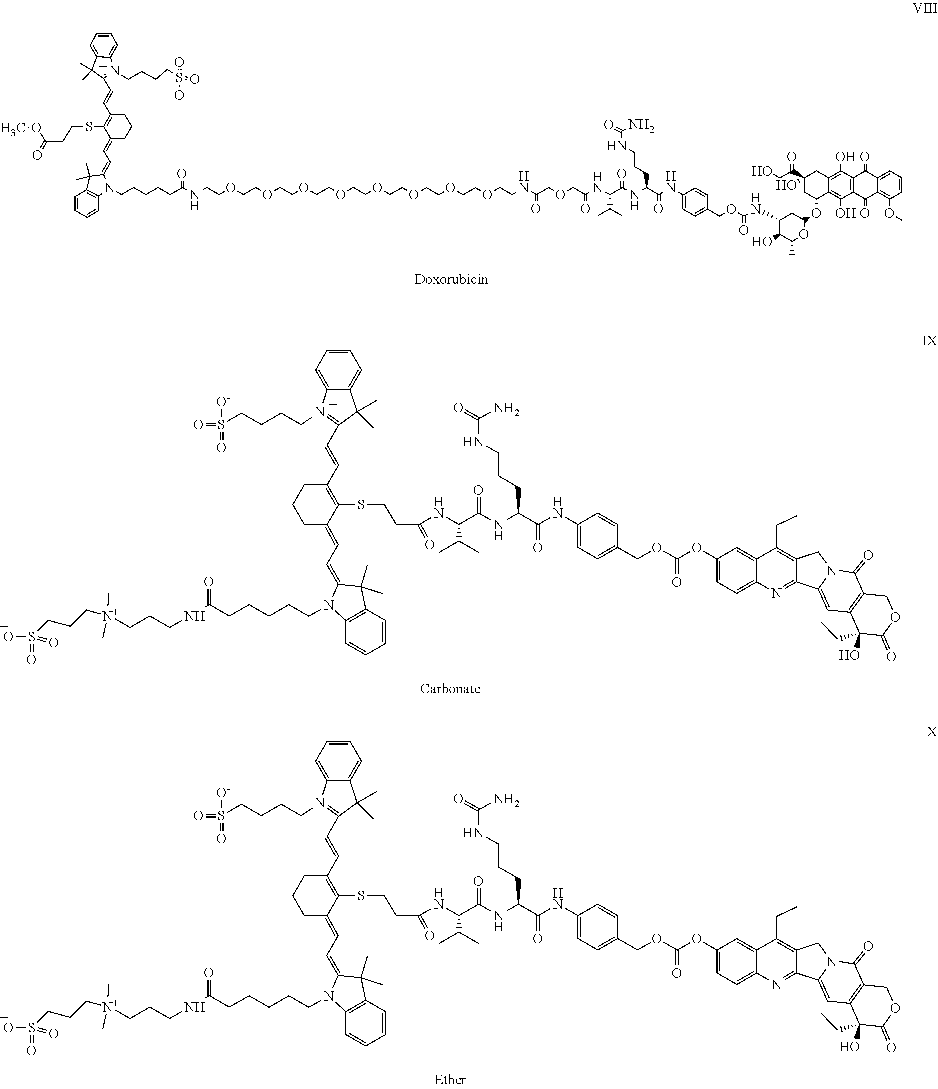

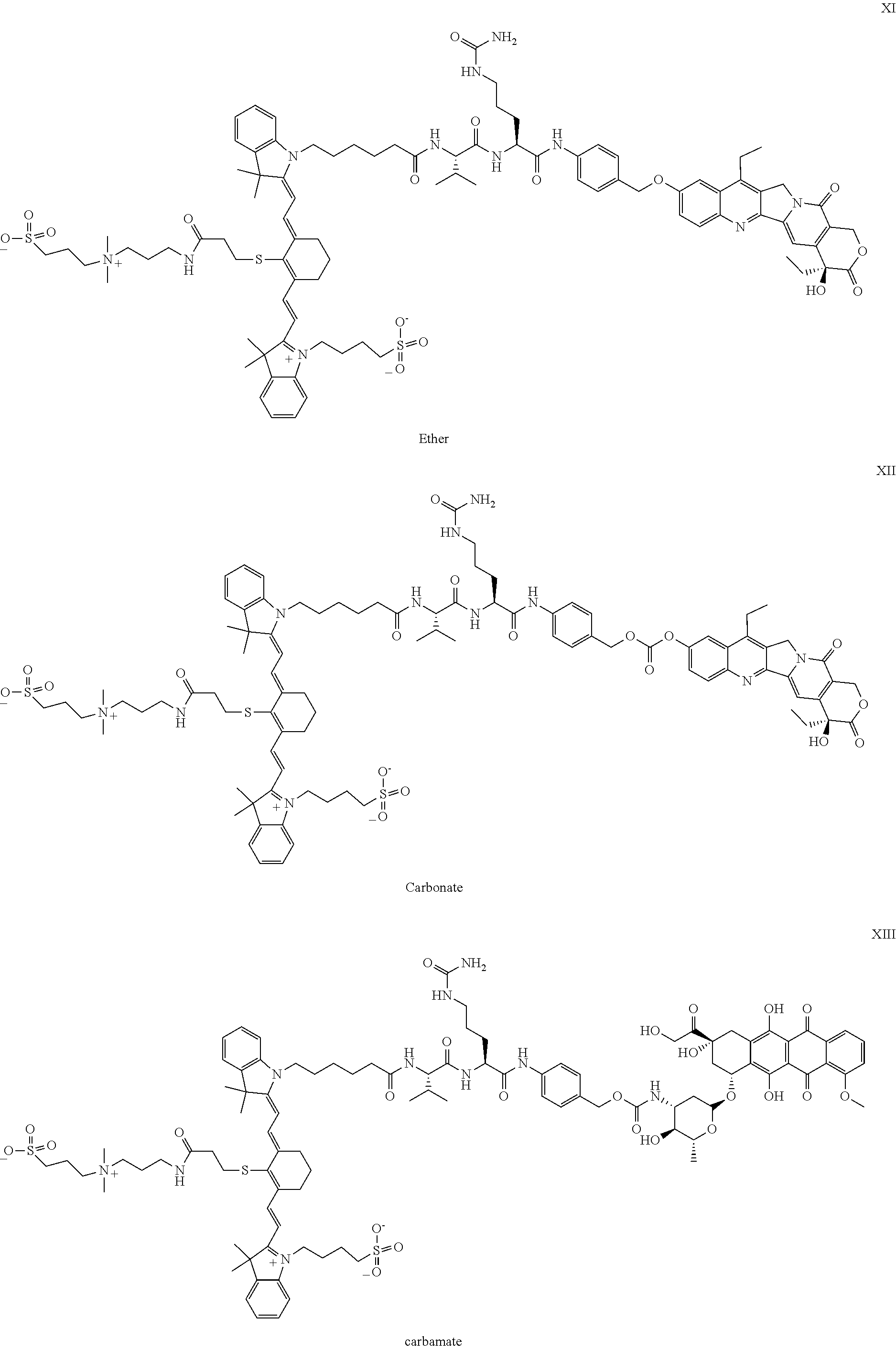

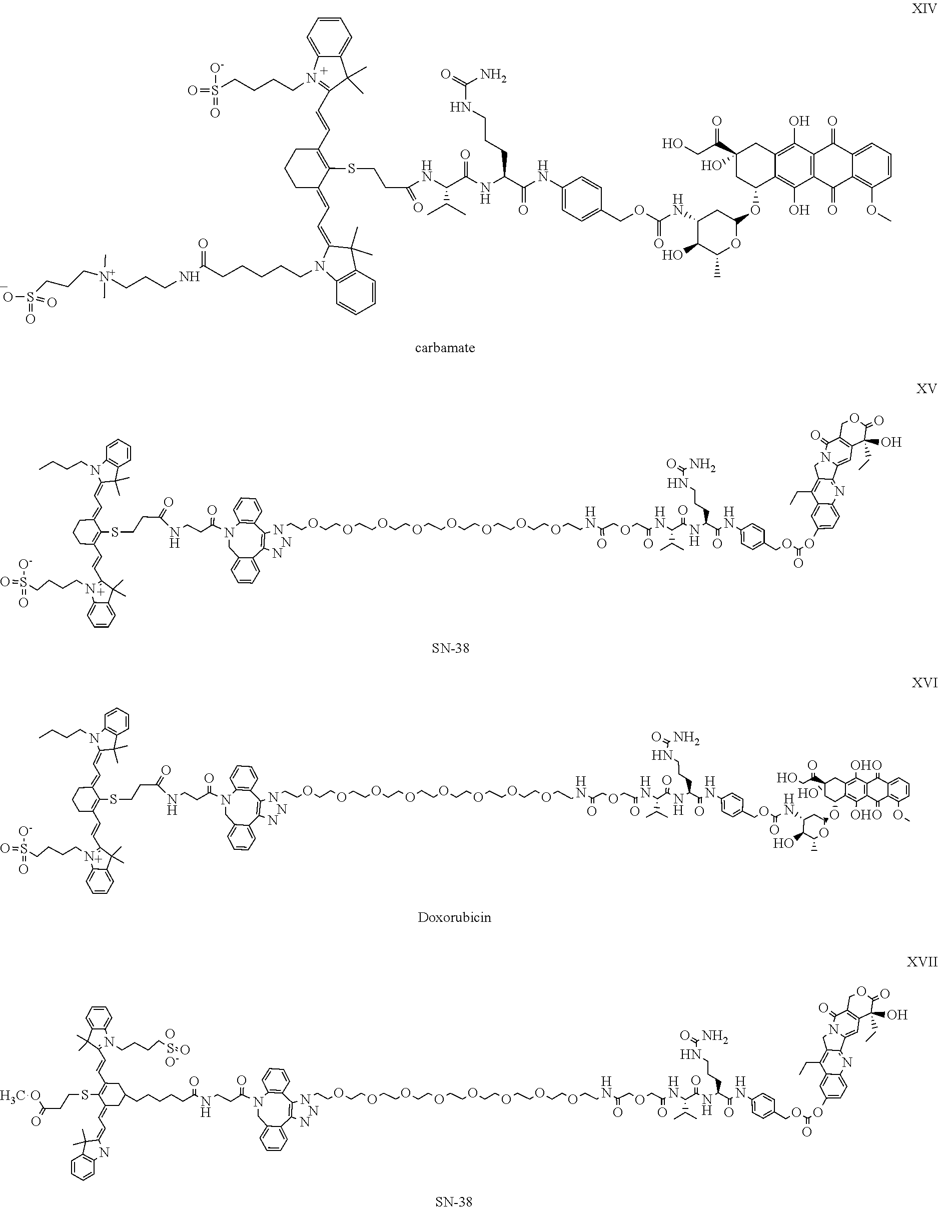

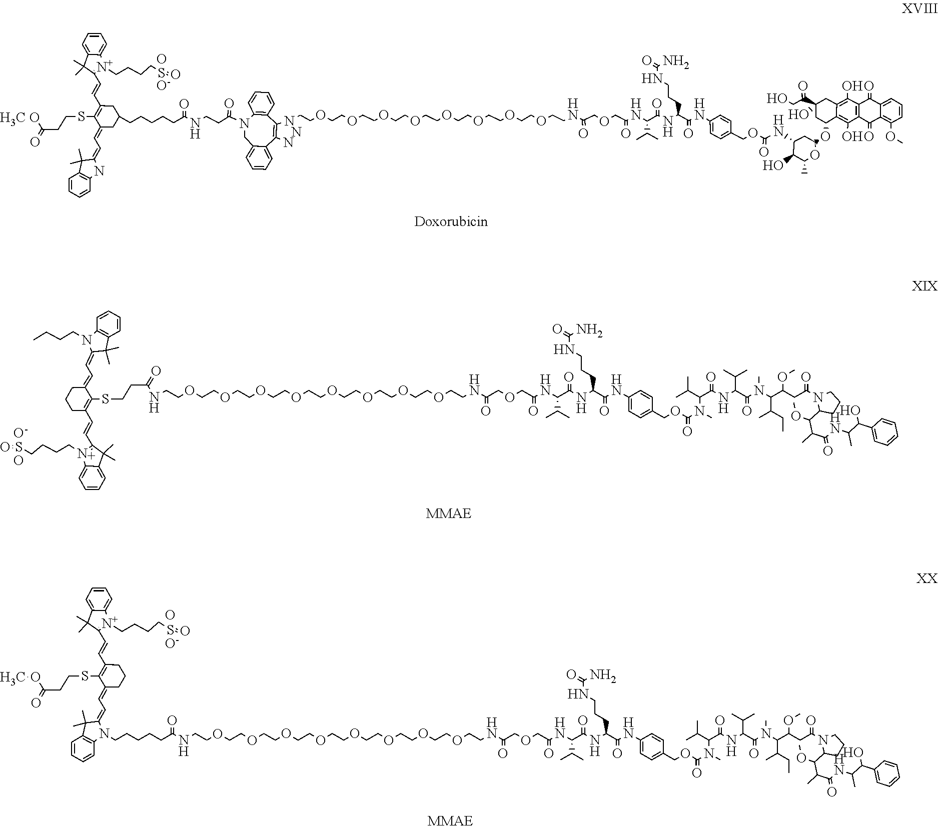

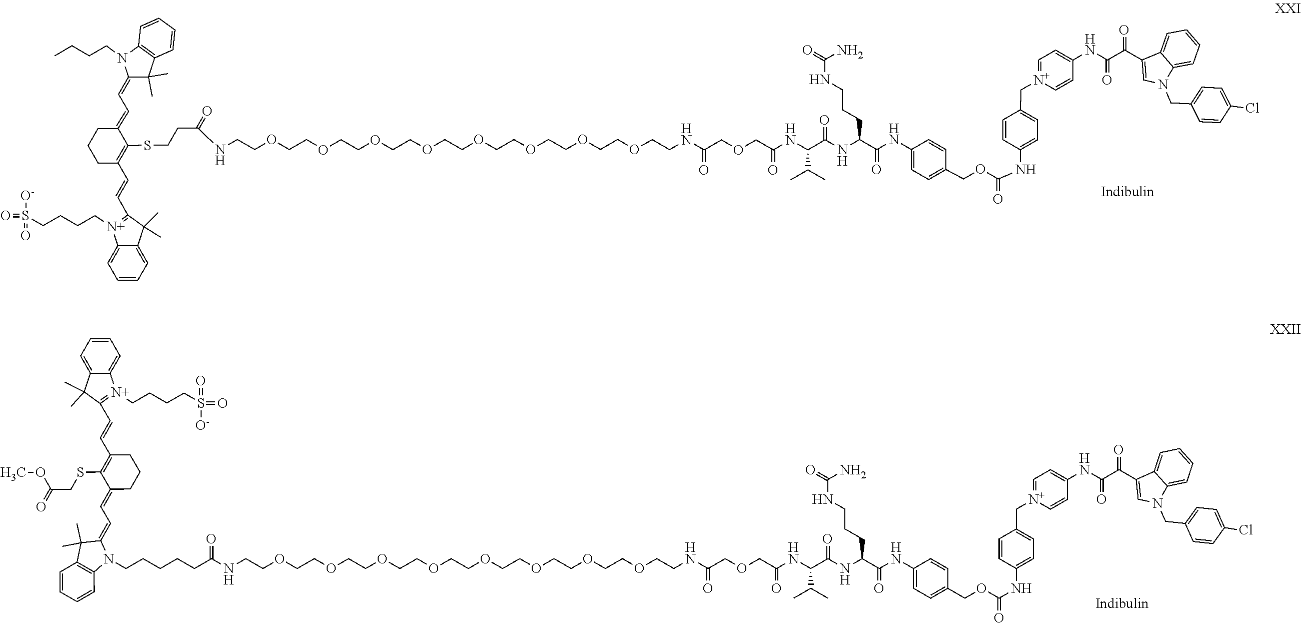

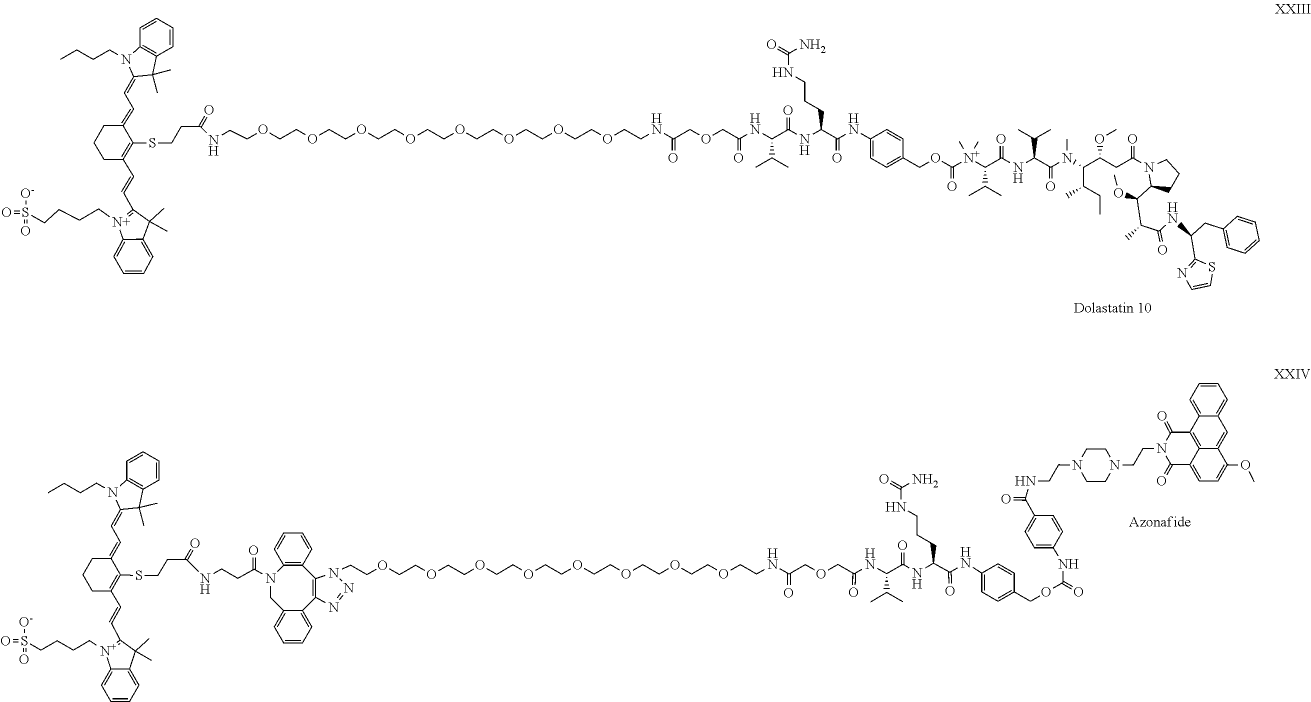

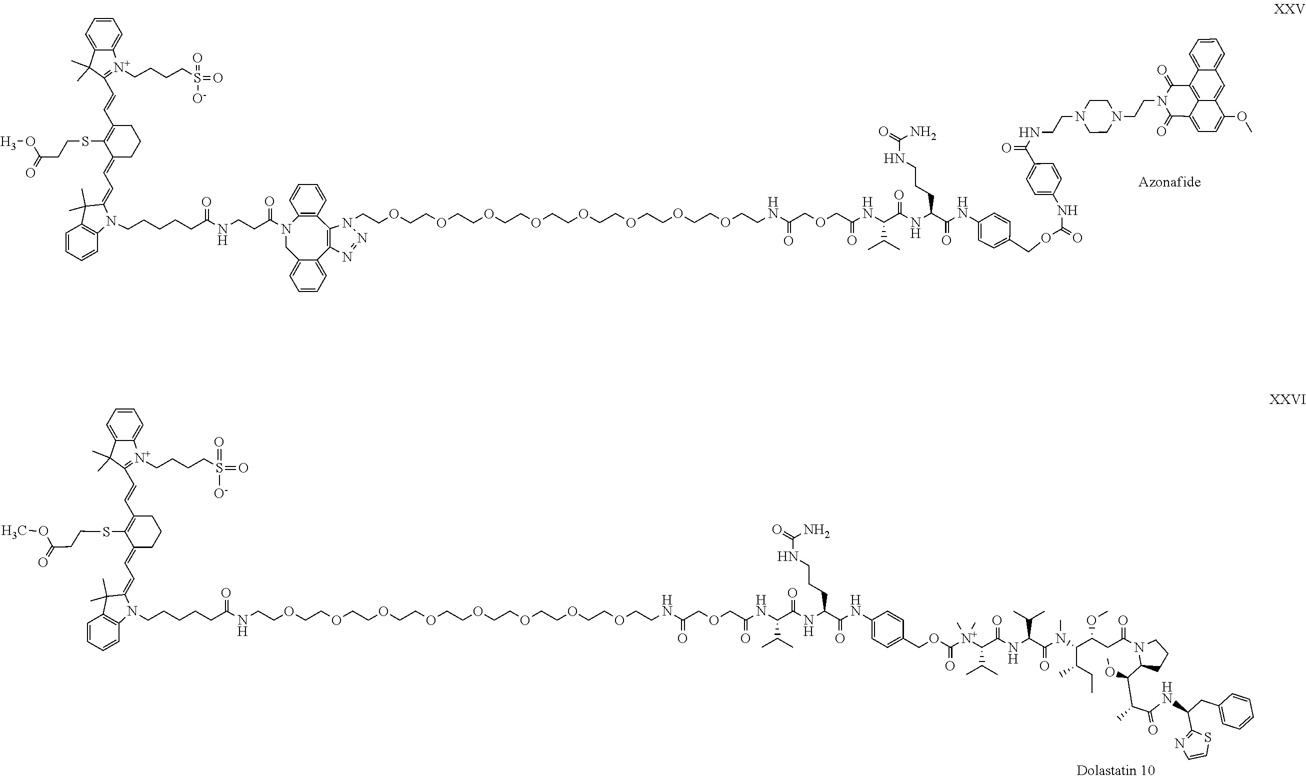

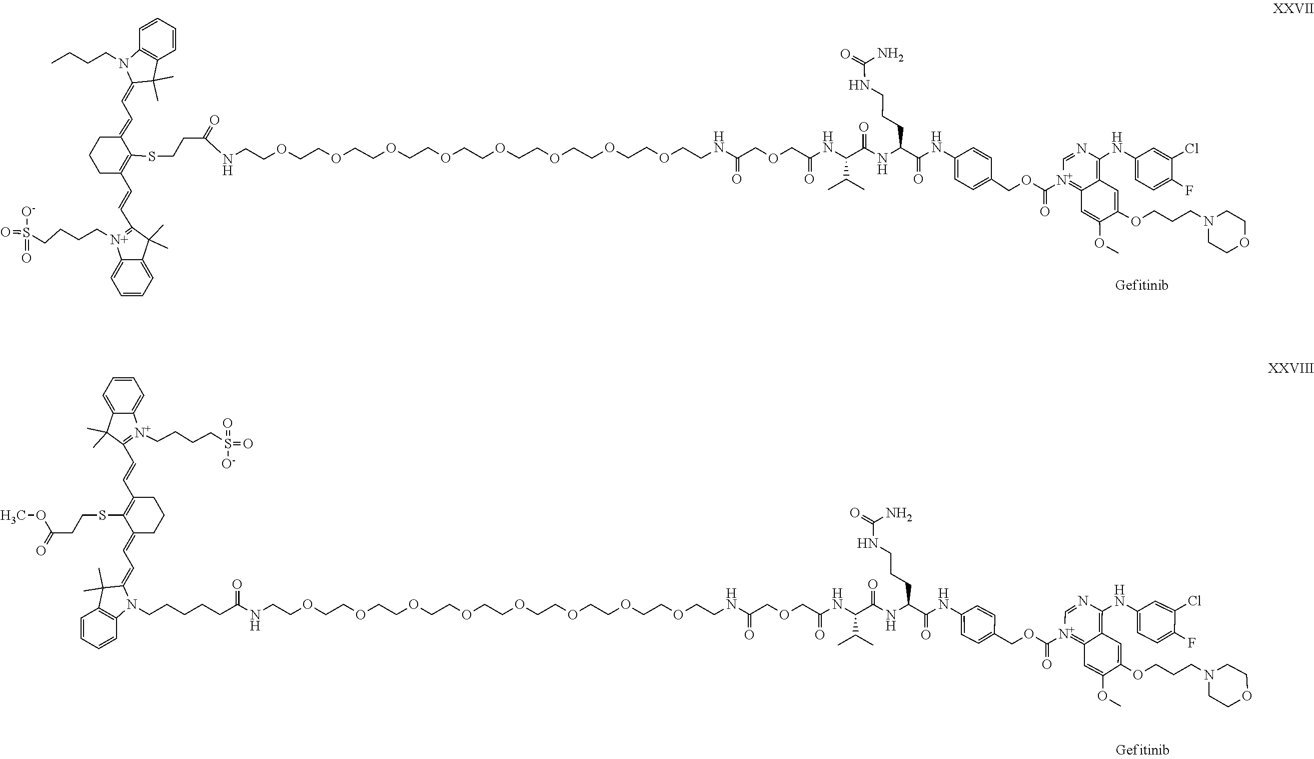

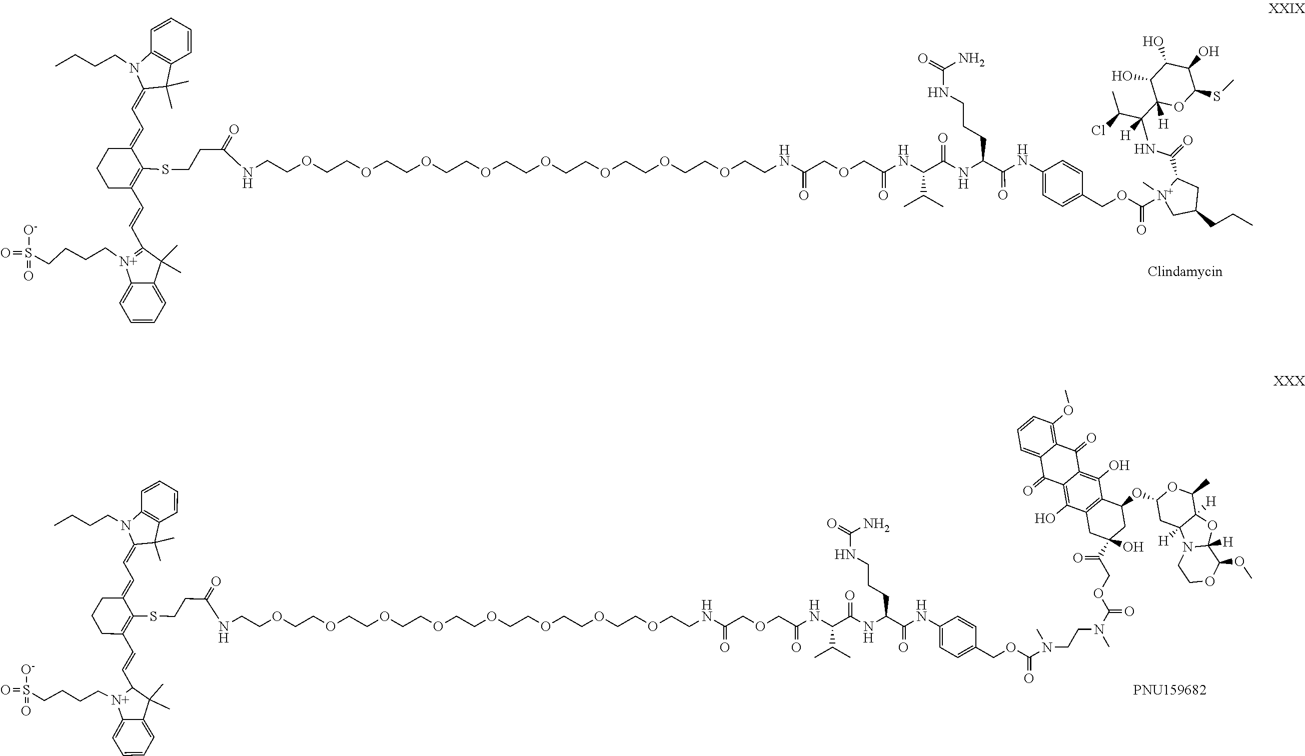

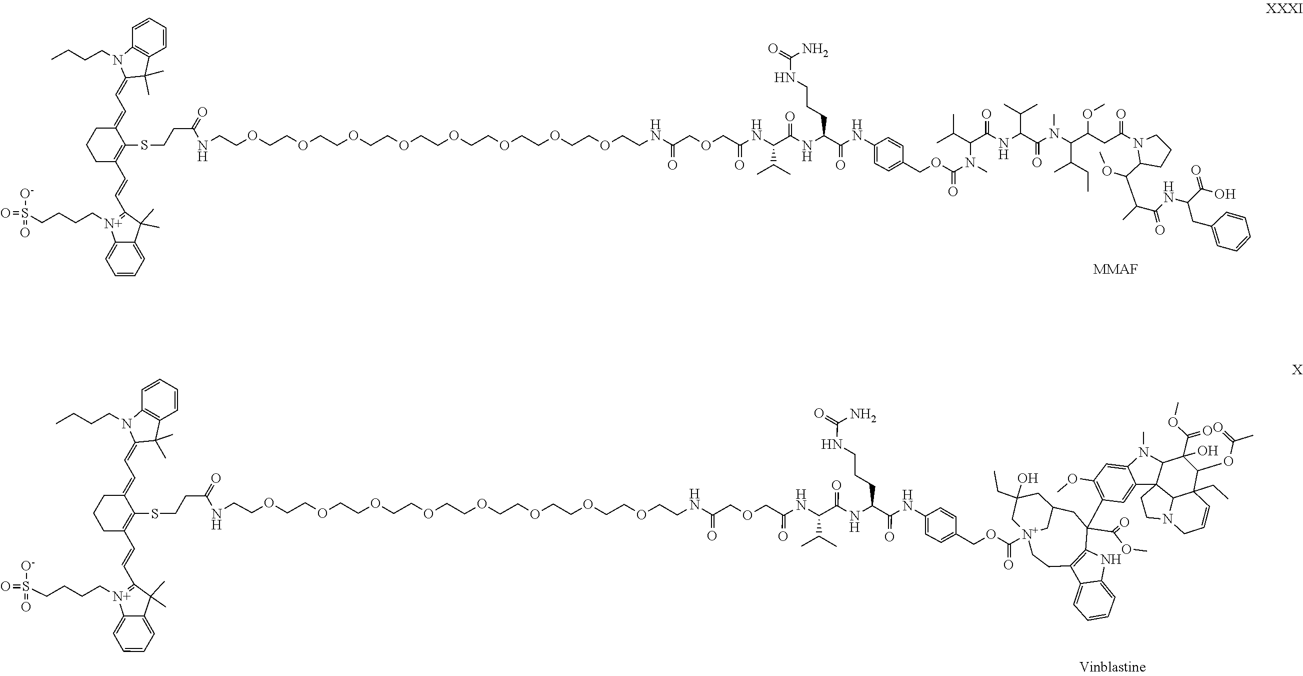

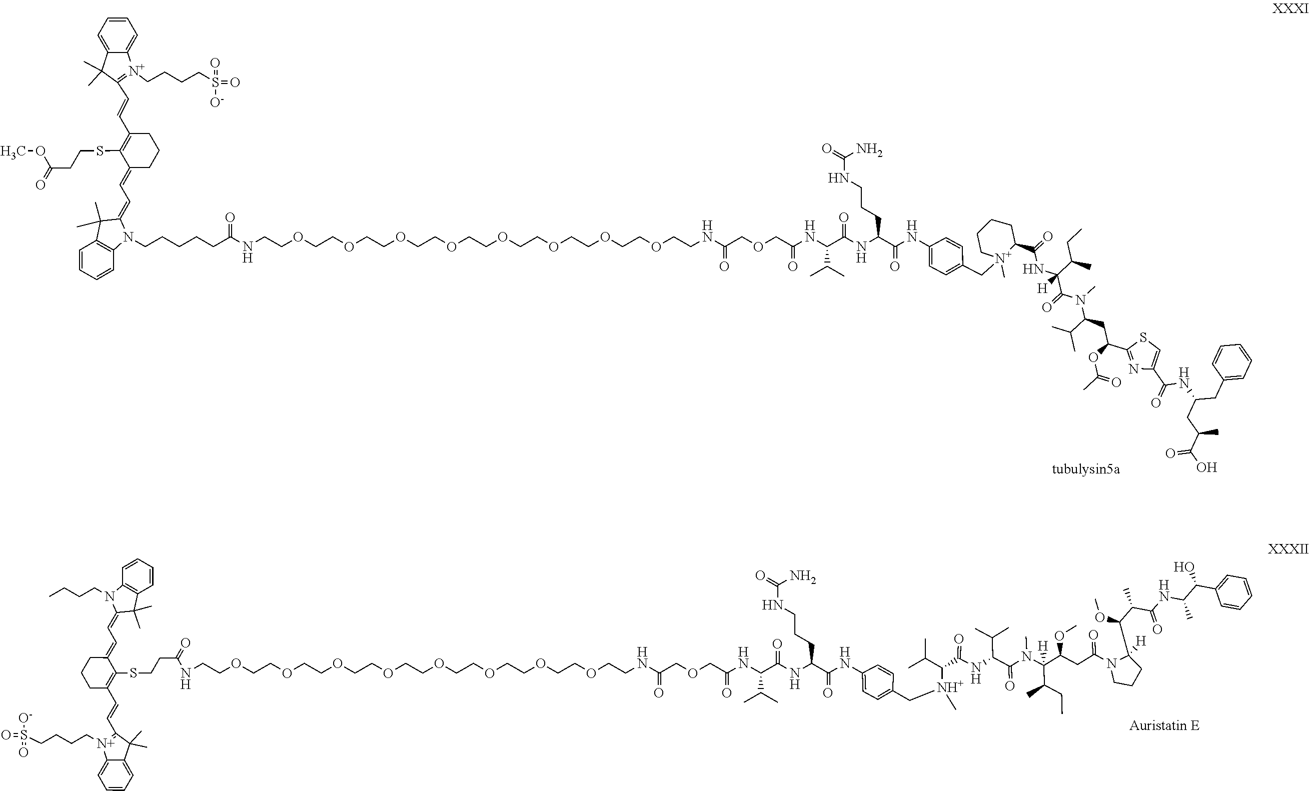

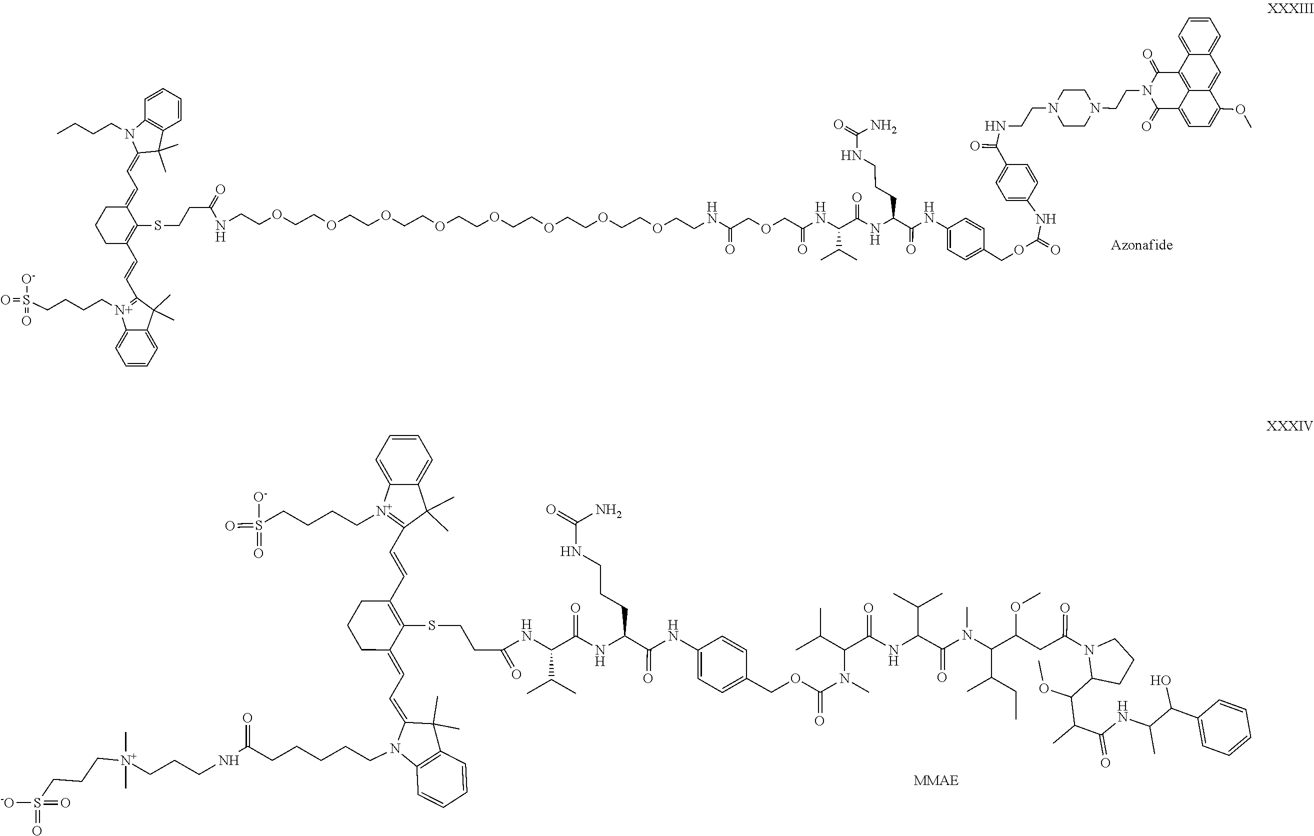

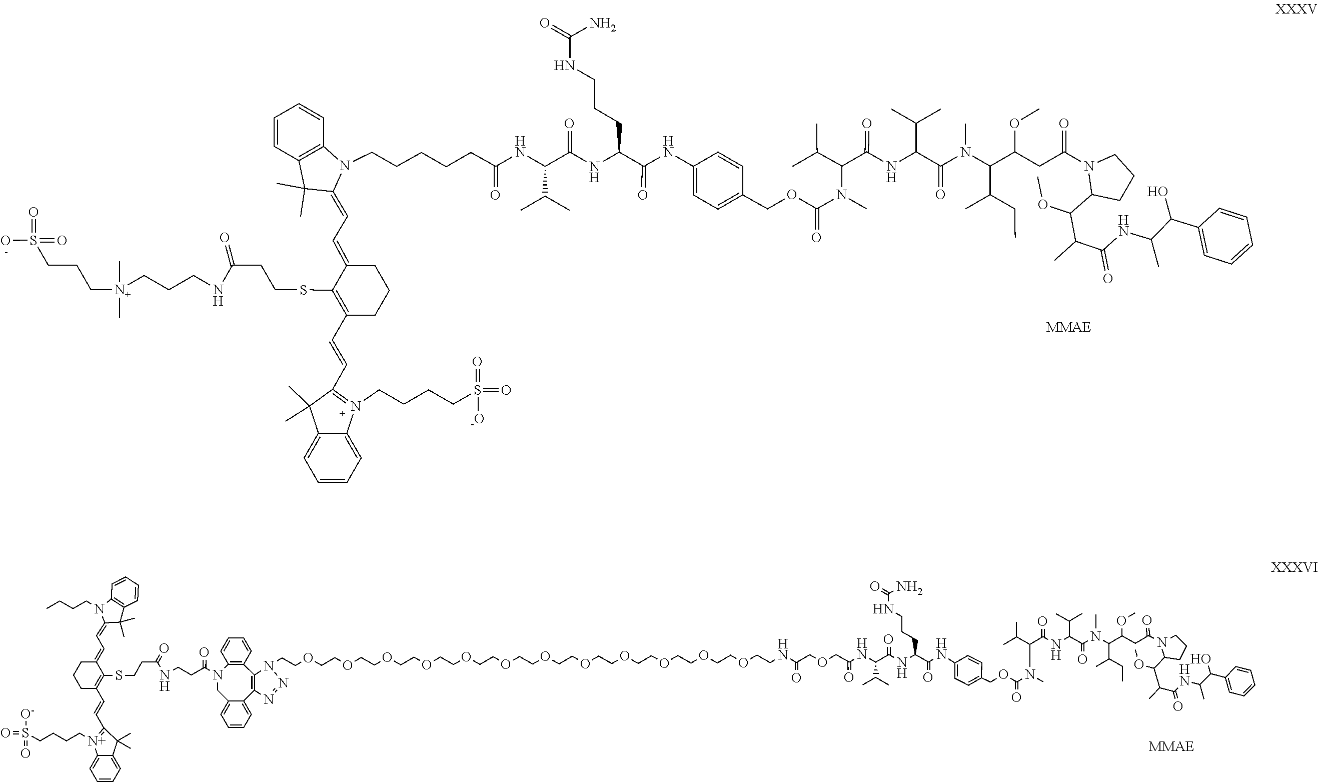

In some embodiments, the dye-drug conjugate compound comprises more than one chemotherapeutic drug coupled to a NIR dye. The use of more than one chemotherapeutic drug may allow for synergistic effects from the different chemotherapy drugs. In some embodiments, the DDC compound comprises a first chemotherapeutic agent and a second chemotherapeutic agent. In some embodiments, the compound comprises four chemotherapeutic drugs. In some embodiments, the compound comprises three chemotherapeutic drugs. In some embodiments, the compound comprises two chemotherapeutic drugs. In some embodiments, the more than one chemotherapeutic drugs are different (e.g., the first chemotherapeutic agent is doxorubicin and the second chemotherapeutic agent is MMAE). In some embodiments, the more than one chemotherapeutic drugs are the same (e.g., the first chemotherapeutic agent is doxorubicin and the second chemotherapeutic agent is doxorubicin).

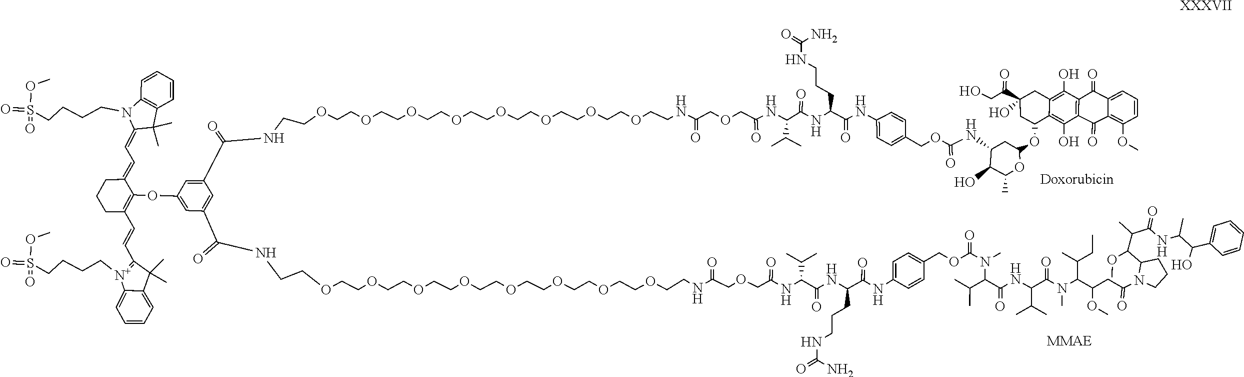

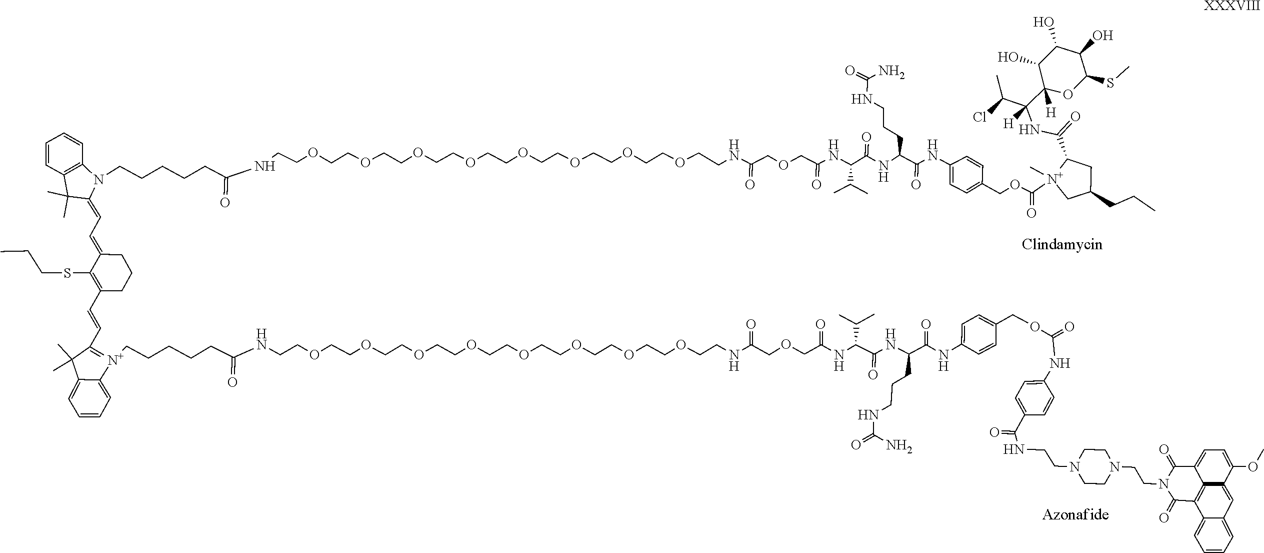

### Further Forms of Compounds Disclosed Herein

### Isomers/Stereoisomers

In some embodiments, the compounds described herein exist as geometric isomers. In some embodiments, the compounds described herein possess one or more double bonds. The compounds presented herein include all cis, trans, syn, anti, entgegen (E), and zusammen (Z) isomers as well as the corresponding mixtures thereof. In some situations, the compounds described herein possess one or more chiral centers and each center exists in the R configuration, or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein. In some embodiments, the compounds described herein are prepared as their individual stereoisomers by reacting a racemic mixture of the compound with an optically active resolving agent to form a pair of diastereoisomeric compounds, separating the diastereomers and recovering the optically pure enantiomers. In some embodiments, dissociable complexes are preferred. In some embodiments, the diastereomers have distinct physical properties (e.g., melting points, boiling points, solubilities, reactivity, etc.) and are separated by taking advantage of these dissimilarities. In some embodiments, the diastereomers are separated by chiral chromatography.

### Labeled Compounds

In some embodiments, the compounds described herein exist in their isotopically-labeled forms. In some embodiments, the methods disclosed herein include methods of treating diseases by administering such isotopically-labeled compounds. In some embodiments, the methods disclosed herein include methods of treating diseases by administering such isotopically-labeled compounds as pharmaceutical compositions. Thus, in some embodiments, the compounds disclosed herein include isotopically-labeled compounds, which are identical to those recited herein, but for the fact that one or more atoms are replaced by an atom having an atomic mass or mass number different from the atomic mass or mass number usually found in nature. Examples of isotopes that can be incorporated into compounds disclosed herein include isotopes of hydrogen, carbon, nitrogen, oxygen, phosphorous, sulfur, fluorine and chloride, such as ²H (D), ³H, ¹³C, ¹⁴C, ¹⁵N, ¹⁸O, ¹⁷O, ³¹P, ³²P, ³⁵S, ¹⁸F, and ³⁶Cl, respectively. Compounds described herein, and the pharmaceutically acceptable salts, solvates, or stereoisomers thereof which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H and carbon-14, i.e., ¹⁴C, isotopes are particularly preferred for their ease of preparation and detectability.

### Pharmaceutically Acceptable Salts

In some embodiments, the compounds described herein exist as their pharmaceutically acceptable salts. In some embodiments, the methods disclosed herein include methods of treating diseases by administering such pharmaceutically acceptable salts. In some embodiments, the methods disclosed herein include methods of treating diseases by administering such pharmaceutically acceptable salts as pharmaceutical compositions.

In some embodiments, the compounds described herein possess acidic or basic groups and therefore react with any of a number of inorganic or organic bases, and inorganic and organic acids, to form a pharmaceutically acceptable salt. In some embodiments, these salts are prepared in situ during the final isolation and purification of the compounds disclosed herein, or a solvate, or stereoisomer thereof, or by separately reacting a purified compound in its free form with a suitable acid or base, and isolating the salt thus formed.

Further, the compounds described herein can be prepared as pharmaceutically acceptable salts formed by reacting the free base form of the compound with a pharmaceutically acceptable inorganic or organic acid.

In some embodiments, those compounds described herein which comprise a free acid group react with a suitable base, such as the hydroxide, carbonate, bicarbonate, sulfate, of a pharmaceutically acceptable metal cation, with ammonia, or with a pharmaceutically acceptable organic primary, secondary, tertiary, or quaternary amine.

### Solvates

In some embodiments, the compounds described herein exist as solvates. The invention provides for methods of treating diseases by administering such solvates. The invention further provides for methods of treating diseases by administering such solvates as pharmaceutical compositions.

Solvates contain either stoichiometric or non-stoichiometric amounts of a solvent, and, in some embodiments, are formed during the process of crystallization with pharmaceutically acceptable solvents such as water, ethanol, and the like. Hydrates are formed when the solvent is water, or alcoholates are formed when the solvent is alcohol. Solvates of the compounds described herein can be conveniently prepared or formed during the processes described herein. By way of example only, hydrates of the compounds described herein can be conveniently prepared by recrystallization from an aqueous/organic solvent mixture, using organic solvents. In addition, the compounds provided herein can exist in unsolvated as well as solvated forms. In general, the solvated forms are considered equivalent to the unsolvated forms for the purposes of the compounds and methods provided herein.

### Tautomers

The compounds described herein include all possible tautomers within the formulas described herein. Tautomers are compounds that are interconvertible by migration of a hydrogen atom, accompanied by a switch of a single bond and adjacent double bond. In bonding arrangements where tautomerization is possible, a chemical equilibrium of the tautomers will exist. All tautomeric forms of the compounds disclosed herein are contemplated. The exact ratio of the tautomers depends on several factors, including temperature, solvent, and pH.

### Methods

Disclosed herein is a method of killing a tumor cell, comprising: administering to said tumor cell a dye-drug conjugate compound comprising:
a tumor-targeting near-infrared dye;
a chemotherapeutic drug; and
a cleavable linker.

In another aspect, provided herein is a method of imaging a tumor cell, comprising: administering to said tumor cell a dye-drug conjugate compound, wherein said dye-drug conjugate compound comprises:
a tumor-targeting near-infrared dye;
a chemotherapeutic drug; and
a cleavable linker.

In some embodiments, said dye-drug conjugate further comprises an optical reporter. In some embodiments, said optical reporter comprises a fluorophore.

In yet another aspect, provided herein is a method of treating a cancer in a subject in need thereof, comprising administering to said subject an effective amount of a dye-drug conjugate compound comprising:
a tumor-targeting near-infrared dye;
a chemotherapeutic drug; and
a cleavable linker.

In some embodiments, said compound is a compound described herein, or a pharmaceutically acceptable salt thereof.

In some embodiments, said compound absorbs and fluoresces in the near-infrared (NIR) region. In some embodiments, said compound absorbs and fluoresces between about 650 to about 1450 nm. In some embodiments, said compound absorbs and fluoresces above 750 nm. In some embodiments, said compound fluoresces above 650 nm. In some embodiments, said compound absorbs and fluoresces above 750 nm. In some embodiments, said compound fluoresces above 750 nm. In some embodiments, said compound absorbs and fluoresces above 800 nm. In some embodiments, said compound fluoresces at about 800 nm.

In some embodiments, said cancer is a solid tumor. In some embodiments, a size of said tumor is reduced by about 1% to about 99%. In some embodiments, said tumor size is reduced by about 1%, about 5%, about 10%, about 20%, about 30%, about 40%, about 50%, about 60%, about 70%, about 80%, or about 90%.

In some embodiments, said cancer is a lung cancer, a breast cancer, a hepatoma, cervical cancer, prostate cancer, leukemia, pancreatic cancer, renal cancer, a glioblastoma, brain cancer, or osteosarcoma. In some embodiments, said cancer comprises lung cancer. In some embodiments, said cancer comprises breast cancer. In some embodiments, said cancer comprises a hepatoma. In some embodiments, said cancer comprises cervical cancer. In some embodiments, said cancer comprises prostate cancer. In some embodiments, said cancer comprises leukemia. In some embodiments, said cancer comprises pancreatic cancer. In some embodiments, said cancer comprises a glioblastoma. In some embodiments, said cancer comprises a brain cancer. In some embodiments, said cancer comprises an osteosarcoma.

### Routes of Administration

Suitable routes of administration include, but are not limited to, oral, intravenous, and parenteral.

In certain embodiments, a compound as described herein is administered in a local rather than systemic manner, for example, via injection of the compound directly into an organ or directly to a tumor, often in a depot preparation or sustained release formulation. In specific embodiments, long-acting formulations are administered by implantation (for example subcutaneously or intramuscularly) or by intramuscular injection. Furthermore, in other embodiments, the drug is delivered in a targeted drug delivery system.

### Pharmaceutical Compositions/Formulations

The compounds described herein are administered to a subject in need thereof, either alone or in combination with pharmaceutically acceptable carriers, excipients or diluents, in a pharmaceutical composition, according to standard pharmaceutical practice. In some embodiments, the compounds described herein are administered to animals.

In another aspect, provided herein are pharmaceutical compositions comprising a compound described herein, or a pharmaceutically acceptable salt or solvate thereof, and at least one pharmaceutically acceptable excipient. A summary of pharmaceutically acceptable excipients that may be used with the compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which are herein incorporated by reference in their entirety.

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### Definitions

Unless defined otherwise, all technical and scientific terms used herein have the same meaning as is commonly understood by one of skill in the art to which this invention belongs. All patents and publications referred to herein are incorporated by reference.

Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

The terms below, as used herein, have the following meanings, unless indicated otherwise:

“Carboxyl” refers to —COOH.

“Cyano” refers to —CN.

“Alkyl” refers to a straight-chain or branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated. In some embodiments, the alkyl is a C₁₋₁₀alkyl. In some embodiments, the alkyl is a C₁₋₆alkyl. In some embodiments, the alkyl is a C₁₋₅alkyl. In some embodiments, the alkyl is a C₁₋₄alkyl. In some embodiments, the alkyl is a C₁₋₃alkyl. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

“Alkoxy” refers to a radical of the formula —ORₐ where Rₐ is an alkyl radical as defined. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system can contain only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. The aryl radical may be a monocyclic, bicyclic, tricyclic or tetracyclic ring system, which may include fused (when fused with a cycloalkyl or heterocycloalkyl ring, the aryl is bonded through an aromatic ring atom) or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Cycloalkyl” refers to a partially or fully saturated, monocyclic or polycyclic carbocyclic ring, which may include fused (when fused with an aryl or a heteroaryl ring, the cycloalkyl is bonded through a non-aromatic ring atom), spiro, or bridged ring systems. In some embodiments, the cycloalkyl is fully saturated. Representative cycloalkyls include, but are not limited to, cycloalkyls having from three to fifteen carbon atoms (C₃-C₁₅ cycloalkyl or C₃-C₁₅ cycloalkenyl), from three to ten carbon atoms (C₃-C₁₀ cycloalkyl or C₃-C₁₀ cycloalkenyl), from three to eight carbon atoms (C₃-C₈ cycloalkyl or C₃-C₈ cycloalkenyl), from three to six carbon atoms (C₃-C₆ cycloalkyl or C₃-C₆ cycloalkenyl), from three to five carbon atoms (C₃-C₅ cycloalkyl or C₃-C₅ cycloalkenyl), or three to four carbon atoms (C₃-C₄ cycloalkyl or C₃-C₄ cycloalkenyl). In some embodiments, the cycloalkyl is a 3- to 10-membered cycloalkyl or a 3- to 10-membered cycloalkenyl. In some embodiments, the cycloalkyl is a 3- to 6-membered cycloalkyl or a 3- to 6-membered cycloalkenyl. In some embodiments, the cycloalkyl is a 5- to 6-membered cycloalkyl or a 5- to 6-membered cycloalkenyl. Monocyclic cycloalkyls include, for example, cyclopropyl, cyclobutyl, cyclopentyl, cyclohexyl, cycloheptyl, and cyclooctyl. Polycyclic cycloalkyls include, for example, adamantyl, norbornyl, decalinyl, bicyclo[3.3.0]octane, bicyclo[4.3.0]nonane, cis-decalin, trans-decalin, bicyclo[2.1.1]hexane, bicyclo[2.2.1]heptane, bicyclo[2.2.2]octane, bicyclo[3.2.2]nonane, and bicyclo[3.3.2]decane, and 7,7-dimethyl-bicyclo[2.2.1]heptanyl. Partially saturated cycloalkyls include, for example cyclopentenyl, cyclohexenyl, cycloheptenyl, and cyclooctenyl. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Halo” or “halogen” refers to bromo, chloro, fluoro or iodo. In some embodiments, halogen is fluoro or chloro. In some embodiments, halogen is fluoro.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro, 2-fluoroethane.

“Hydroxyalkyl” refers to an alkyl radical, as defined above, that is substituted by one or more hydroxyls. In some embodiments, the alkyl is substituted with one hydroxyl. In some embodiments, the alkyl is substituted with one, two, or three hydroxyls. Hydroxyalkyl include, for example, hydroxymethyl, hydroxyethyl, hydroxypropyl, hydroxybutyl, or hydroxypentyl. In some embodiments, the hydroxyalkyl is hydroxymethyl.

“Aminoalkyl” refers to an alkyl radical, as defined above, that is substituted by one or more amines. In some embodiments, the alkyl is substituted with one amine. In some embodiments, the alkyl is substituted with one, two, or three amines. Aminoalkyl include, for example, aminomethyl, aminoethyl, aminopropyl, aminobutyl, or aminopentyl. In some embodiments, the aminoalkyl is aminomethyl.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃) OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Heterocycloalkyl” refers to a 3- to 24-membered partially or fully saturated ring radical comprising 2 to 23 carbon atoms and from one to 8 heteroatoms selected from the group consisting of nitrogen, oxygen, phosphorous, silicon, and sulfur. In some embodiments, the heterocycloalkyl is fully saturated. In some embodiments, the heterocycloalkyl comprises one to three heteroatoms selected from the group consisting of nitrogen, oxygen, and sulfur. In some embodiments, the heterocycloalkyl comprises one to three heteroatoms selected from the group consisting of nitrogen and oxygen. In some embodiments, the heterocycloalkyl comprises one to three nitrogens. In some embodiments, the heterocycloalkyl comprises one or two nitrogens. In some embodiments, the heterocycloalkyl comprises one nitrogen. In some embodiments, the heterocycloalkyl comprises one nitrogen and one oxygen. Unless stated otherwise specifically in the specification, the heterocycloalkyl radical may be a monocyclic, bicyclic, tricyclic or tetracyclic ring system, which may include fused (when fused with an aryl or a heteroaryl ring, the heterocycloalkyl is bonded through a non-aromatic ring atom), spiro, or bridged ring systems; and the nitrogen, carbon, or sulfur atoms in the heterocycloalkyl radical may be optionally oxidized; the nitrogen atom may be optionally quaternized. Representative heterocycloalkyls include, but are not limited to, heterocycloalkyls having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₈ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₇ heterocycloalkenyl), from two to five carbon atoms (C₂-C₅ heterocycloalkyl or C₂-C₈ heterocycloalkenyl), or two to four carbon atoms (C₂-C₄ heterocycloalkyl or C₂-C₄ heterocycloalkenyl). Examples of such heterocycloalkyl radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycloalkyl also includes all ring forms of the carbohydrates, including but not limited to the monosaccharides, the disaccharides and the oligosaccharides. In some embodiments, heterocycloalkyls have from 2 to 10 carbons in the ring. It is understood that when referring to the number of carbon atoms in a heterocycloalkyl, the number of carbon atoms in the heterocycloalkyl is not the same as the total number of atoms (including the heteroatoms) that make up the heterocycloalkyl (i.e., skeletal atoms of the heterocycloalkyl ring). In some embodiments, the heterocycloalkyl is a 3- to 8-membered heterocycloalkyl. In some embodiments, the heterocycloalkyl is a 3- to 7-membered heterocycloalkyl. In some embodiments, the heterocycloalkyl is a 3- to 6-membered heterocycloalkyl. In some embodiments, the heterocycloalkyl is a 4- to 6-membered heterocycloalkyl. In some embodiments, the heterocycloalkyl is a 5- to 6-membered heterocycloalkyl. In some embodiments, the heterocycloalkyl is a 3- to 8-membered heterocycloalkenyl. In some embodiments, the heterocycloalkyl is a 3- to 7-membered heterocycloalkenyl. In some embodiments, the heterocycloalkyl is a 3- to 6-membered heterocycloalkenyl. In some embodiments, the heterocycloalkyl is a 4- to 6-membered heterocycloalkenyl. In some embodiments, the heterocycloalkyl is a 5- to 6-membered heterocycloalkenyl. Unless stated otherwise specifically in the specification, a heterocycloalkyl may be optionally substituted as described below, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycloalkyl is optionally substituted with oxo, halogen, methyl, ethyl, —CN, —COOH, COOMe, —CF₃, —OH, —OMe, —NH₂, or —NO₂. In some embodiments, the heterocycloalkyl is optionally substituted with halogen, methyl, ethyl, —CN, —CF₃, —OH, or —OMe. In some embodiments, the heterocycloalkyl is optionally substituted with halogen.

“Heteroaryl” refers to a 5- to 14-membered ring system radical comprising one to thirteen carbon atoms, one to six heteroatoms selected from the group consisting of nitrogen, oxygen, phosphorous, and sulfur, and at least one aromatic ring. In some embodiments, the heteroaryl comprises one to three heteroatoms selected from the group consisting of nitrogen, oxygen, and sulfur. In some embodiments, the heteroaryl comprises one to three heteroatoms selected from the group consisting of nitrogen and oxygen. In some embodiments, the heteroaryl comprises one to three nitrogens. In some embodiments, the heteroaryl comprises one or two nitrogens. In some embodiments, the heteroaryl comprises one nitrogen. The heteroaryl radical may be a monocyclic, bicyclic, tricyclic or tetracyclic ring system, which may include fused (when fused with a cycloalkyl or heterocycloalkyl ring, the heteroaryl is bonded through an aromatic ring atom) or bridged ring systems; and the nitrogen, carbon or sulfur atoms in the heteroaryl radical may be optionally oxidized; the nitrogen atom may be optionally quaternized. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

The term “one or more” when referring to an optional substituent means that the subject group is optionally substituted with one, two, three, or four substituents. In some embodiments, the subject group is optionally substituted with one, two, or three substituents. In some embodiments, the subject group is optionally substituted with one or two substituents. In some embodiments, the subject group is optionally substituted with one substituent. In some embodiments, the subject group is optionally substituted with two substituents.

The term “salt” or “pharmaceutically acceptable salt” refers to salts derived from a variety of organic and inorganic counter ions known in the art. Pharmaceutically acceptable acid addition salts can be formed with inorganic acids and organic acids.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient.

An “effective amount” or “therapeutically effective amount” refers to an amount of a compound administered to a mammalian subject, either as a single dose or as part of a series of doses, which is effective to produce a desired therapeutic effect.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition.

### EXAMPLES

The following examples are offered to illustrate, but not to limit the claimed invention. The following examples further illustrate the invention but, of course, should not be construed as in any way limiting its scope.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### Example 1. Synthesis of Exemplary Dye-Drug Conjugates

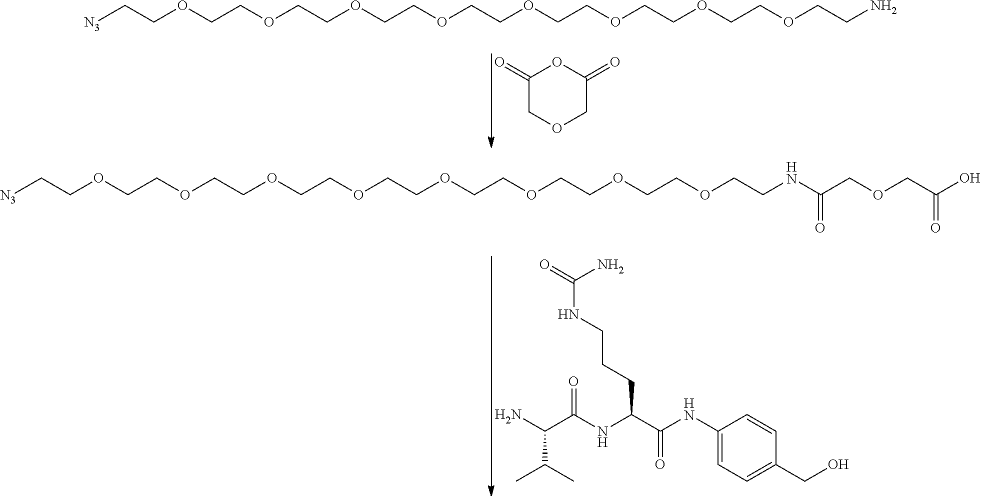

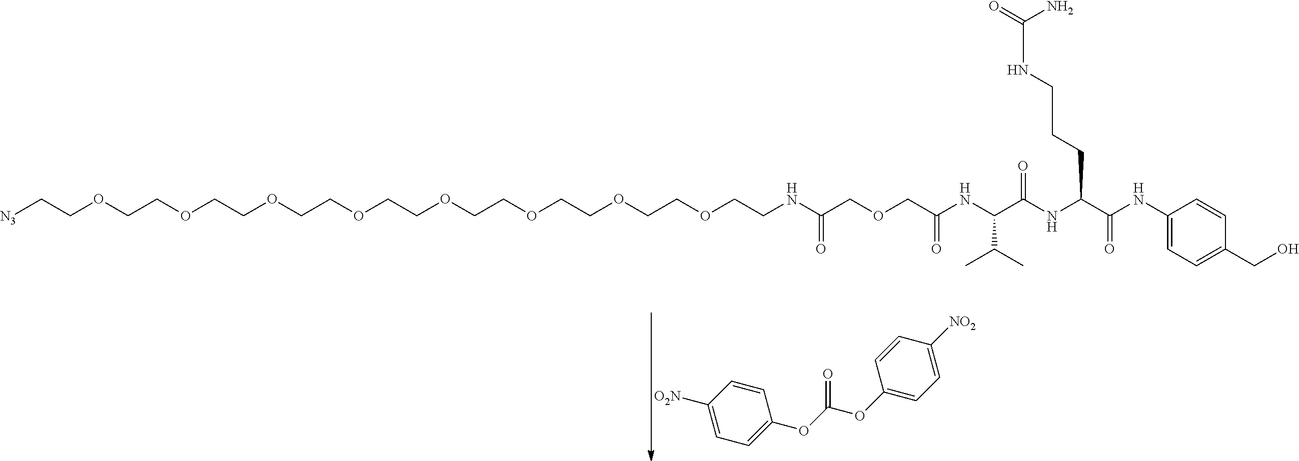 [SMILES: CC(C)[C@H](NC(=O)COCC(=O)NCCOCCOCCOCCOCCOCCOCCOCCOCCN=[N+]=[N-])C(=O)N[C@@H](CCCNC(N)=O)C(=O)Nc1ccc(CO)cc1 ; O=C(Oc1ccc([N+](=O)[O-])cc1)Oc1ccc([N+](=O)[O-])cc1]

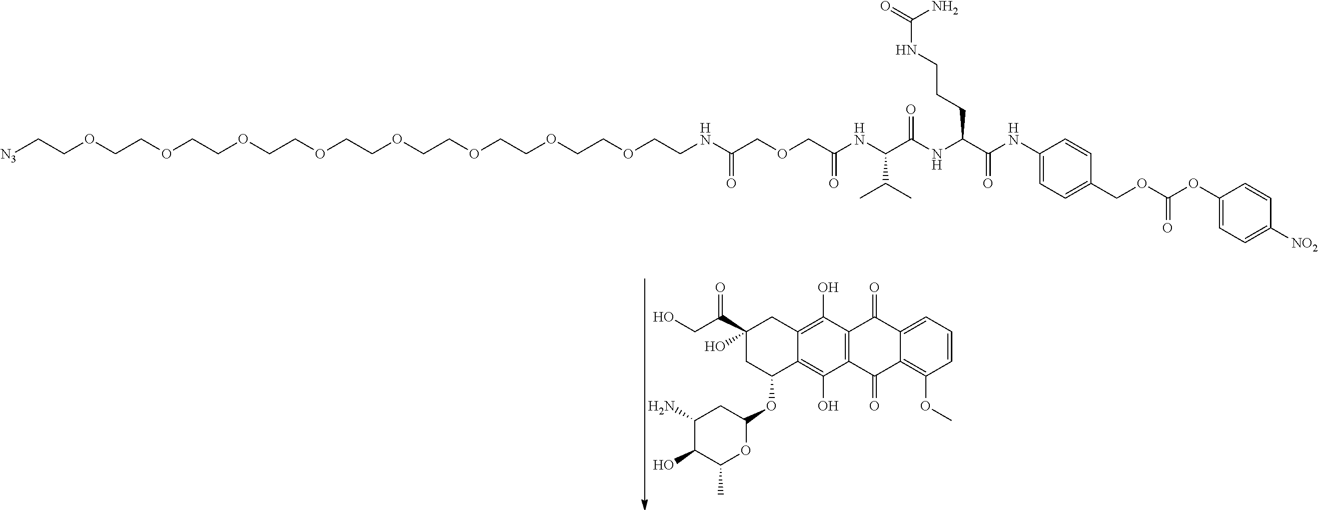

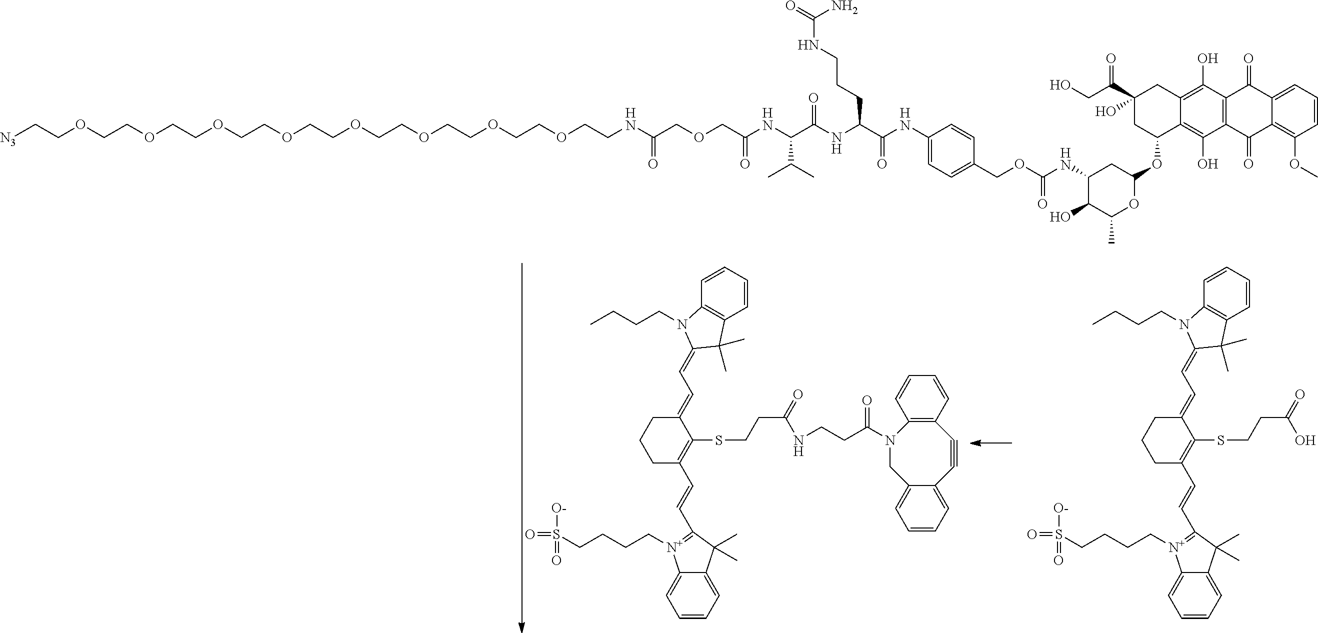

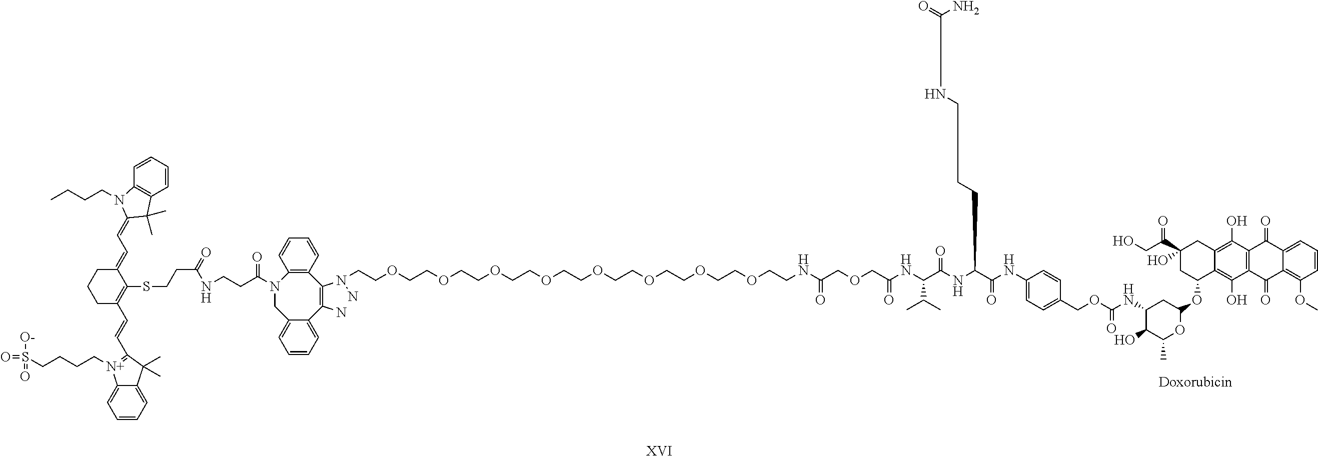

Step 1. To a solution of N₃-(PEG)₈-NH₂ (1 eq) in dichloromethane was added diglycolic anhydride (1.5 eq) and the reaction mixture was stirred overnight. The solution was concentrated to give a yellowish residue which was dissolved in water (250 mL). The product was isolated from the aqueous phase by continuous extraction with dichloromethane overnight. The product was obtained in 85% and ESI-MS and NMR analysis confirm the product. The product was used in the next step without further purification.

Step 2. Val-Cit-PAB (1000 mg, 2.635 mmol, 1 eq) was dissolved in 20 mL of DMF. DIPEA (1.1 eq), HBTU (1.1 eq) and the acid derivative obtained from step 1 (1.1 eq) were added the reaction was stirred overnight. The crude was purified by chromatography (silica gel, DCM/methanol mixture) to yield the desired product.

Step 3. A screw-cap vial was charged with N₃-(PEG)₈-VC-PAB obtained from step 2 (1.0 eq.), bis(4-nitrophenyl) carbonate (1.0 eq.) and DIPEA (4.0 eq.) in DMF and the reaction was stirred at room temperature for 1 h. The reaction mixture was poured into ice-cold ether and the filtrate was collected via centrifugation and dried under vacuum to obtain the desired product as a yellowish powder. Analytical data was in accordance with previously reported results.

Step 4. A screw-cap vial was charged with doxorubicin (1.0 eq.) in DMSO, N₃-(PEG)₈-VC-PAB-PNP obtained from Step 3 (1.2 eq.) in DMSO solution and a solution of HOBT hydrate in DMSO (1.0 eq.). Then DIPEA (3.0 eq.) was added and the resulting reaction was stirred for three h at room temperature. After the consumption of the doxorubicin starting material, monitored via TLC, a mixture of acetonitrile and water was added. The crude product was purified via preparative HPLC. The desired product was obtained and its analytical data was in agreement with the desired structure.

Step 5. HOBt hydrate (2 eq.), EDC HCl (1.6 eq.) and dibenzocyclooctyne (DBCO) amine (1.0 eq.) were added to a solution of NIR dye 1 (1 eq.) in dry DMF and Stir the mixture was stirred for 20 h at room temperature. The reaction mixture was concentrated in vacuo and the residue was suspended in DCM. The mixture was washed with sat. NH₄Cl and brine, dried over Na₂SO₄, and concentrated. The crude product was purified with a SiO₂ column (using a mixture of DCM and methanol) to obtain the desired product.

Step 6. DBCO-functionalized NIR dye (1 eq.) was added to azide functionalized doxorubicin drug (1.5 eq.) in a mixture of acetonitrile and water. The result solution was stirred overnight at 4° C. The solution was concentrated in vacuo and the crude product was purified with a SiO₂ column (using a mixture of DCM and methanol) to obtain the desired product XVI. Its analytical data including HPLC/mass (FIGS. and ¹HNMR agreed with the desired structure with 96.4% purity.

DDC compounds were synthesized in a manner similar to Example 1. Analytical data is listed below in Table 5.

TABLE 5

| | MW | HPLC-ELSD |
|---|---|---|
| DDC Compound | [M + 1] | (purity) |
| Dye2-triazole-PEG8-Val- | 2533.0 | 96.4% |
| Cit-PABC-Doxorubicin | | |
| Dye2-triazole-PEG8-Val- | 2381.9 | 96.6% |
| Cit-PABC-SN-38 | | |
| Dye1-PEG8-Val-Cit-PABA- | 2460.9 | 99.1% |
| Doxorubicin | | |

### Example 2. Toxicity and Tumor Targeting Ability

### In Vitro Antitumor Activities

The growth inhibition of the dye1 (Table 5) dye1-SN38 (XV), dye1-doxo (XVI), dye2 (Table 5), dye2-SN38 (XVII), dye2-doxo (XVIII) were determined by cell viability assay. Three cancer cell lines A549 (lung cancer), MDA-MB-231 (breast cancer), and SW620 (colon cancer) were incubated at 37° C. in a humidified atmosphere of 5% CO₂. Cells were harvested and seeded in 96 well culture plates at a density of 5×10³ cells/well in 100 μl media/10% FBS and incubated for overnight at 37° C., 5% CO₂.

After overnight culture, the media was replaced with 100 μL of fresh medium/10% FBS, and the compounds were added at a 2.5-fold serial dilution concentration (μM): 20 μM, 8 μM, 3.2 μM, 1.28 μM, 0.512 μM, 205 nM, 82 nM, 0 (8 concentrations total).

Cells were incubated with compounds for 24, 48, and 72 h at 37° C., 5% CO₂. The plate was equilibrated at room temperature for approximately 30 min.

Then 100 μL of the Cell Titer Glo labeling reagent was added to each well.

The plate was mixed for 2 minutes on an orbital shaker to induce cell lysis.

Then the plate was incubated at room temperature for 10 minutes to stabilize the luminescent signal.

Cell viability assay was measured using Cell Titer-Glo luminescent cell ability kit via two parameters: Hoechst nuclei counts via imaging, and 2D Celltiter-glo measuring ATP content from live cells. IC50 values were calculated with a 4-variable curve analysis.

### In Vitro Cell Imaging

The tumor cells were seeded in a chamber and incubated for 24 h at 37° C. under 5% CO₂. After removal of medium, cells were treated with the test materials at 5 μM concentration. When reaching the time points, cells were washed with PBS three times, then fluorescent images were taken under a fluorescent microscope.

To illustrate the tumor-targeting ability of the compounds, Dye was used in 2D cancer cell studies. Five types of cancer cells were dosed with 20 μM of said dye (absorbance of ˜780 nm, emission of ˜808 nm) and the NIR intensity mean per well over time correlating to dye uptake is shown in FIG. 5 for a lung cancer cell line (A549) versus four normal cell lines (colon cells, kidney cells, liver cells, and human umbilical vein endothelial cells (HUVEC)). It is evident that significant dye uptake is only in the cancer cell line (black line for A549), while minimum dye uptake is observed in all other normal cells

The tumor-targeting ability and therapeutic efficacy of a four additional DDC prodrugs together with the controls of two corresponding NIR dyes, as well as two chemotherapeutic drugs were explored (Table 6) in 2D lung cancer cell line (A549) studies.

TABLE 6
Compounds used in the 2D cell studies.

| | | | XVII | XVIII |
|---|---|---|---|---|
| | XV | XVI | Dye 2- | Dye 2- |
| DDC | Dye 1-SN38 | Dye 1-Doxo | SN38 | Doxo |
| Con- trols | 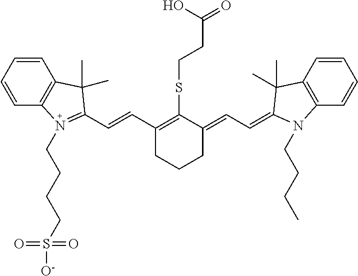 | 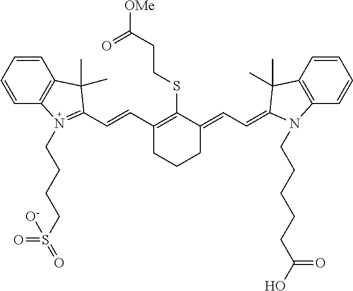 | SN-38 | Doxo- rubicin |
| | Dye 1 | Dy2 | | |

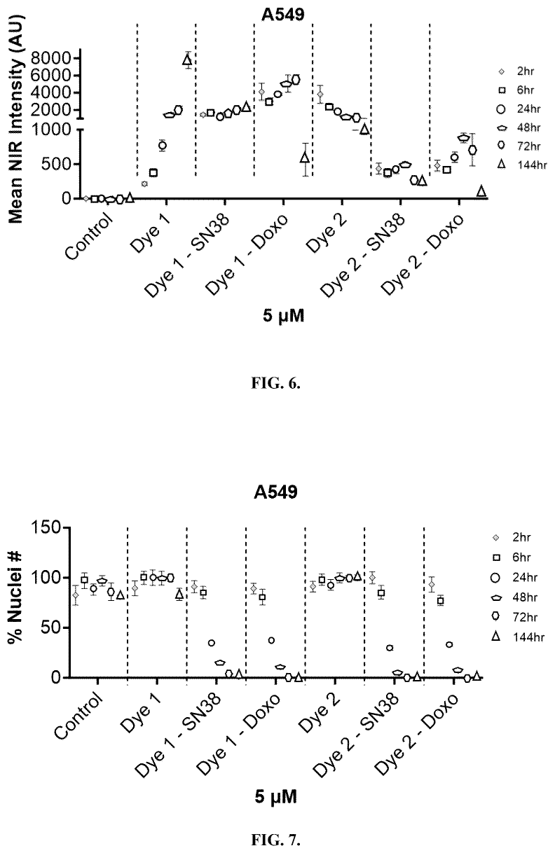

The cell lines were dosed with 5 μM of said compounds. Two studies were carried out: one was the compounds uptake study by the tumor cell line as judged by both the NIR intensity mean (FIG. 6) and by nuclei counts (FIG. 7) per well over time.

The NIR Mean Intensity data from FIG. 6 showed that the two Dye 1 based prodrugs Dye 1-SN38 and Dye 1-Doxo were taken up well in the lung cancer cell line as compared with the two control dyes Dye 1 and Dye 2 across all time points.

FIG. 7 shows the uptake data by Nuclei Counts. All three controls: blank (Control), Dye 1, and Dye 2 are not toxic over all the time points, as expected, and showed similar behavior. However, all four prodrugs behaved differently from the controls. For the first two time points 2 hours and 6 hours, almost no nuclei died, indicating that all four prodrugs are not toxic, similar to the controls. At 24 hour time point, more than half of nuclei died and at 48 hours and beyond, all nuclei died, indicating these prodrugs become toxic to tumor cells.

The prodrugs were stable initially as non-toxic prodrug forms, upon longer exposure, they started to be cleaved by the enzyme present in these tumor cells to release the drug doxorubicin or SN-38, which are toxic to the cells.

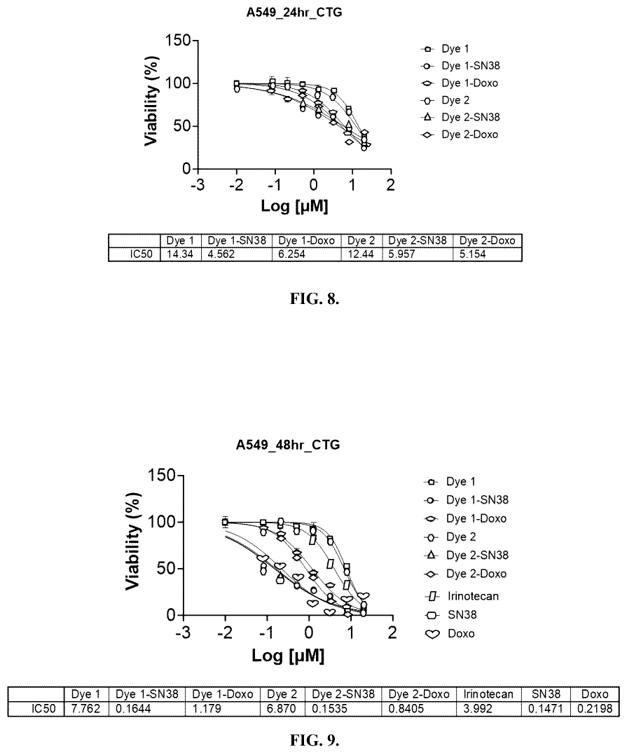

The toxicity study of each of these compounds in the cell line was also carried out as judged by both the cell viability in percentage and IC₅₀ data at two time points of 24 hours (FIG. 8) and 48 hours (FIG. 9) after the cells were dosed.

The toxicity study results show that at 48 hours all four prodrugs are more toxic than they were at 24 hours, as judged by both cell viability and IC₅₀. In fact, the IC₅₀ of all four prodrugs were close to their parent (unconjugated and therefore toxic) drugs SN38 and Doxorubicin. The results shows that the prodrugs were cleaved to release the parent drug molecules once they are localized in the tumor cells, and the releasing (cleaving) process requires between 1-3 days.

### Example 3. In Vivo Animal Experiment

All studies were conducted in compliance with accepted standards for the care and use of laboratory animals, including the following: 1) Animal Welfare Act Regulations (9 CFR); 2) U.S. Public Health Service Office of Laboratory Animal Welfare (OLAW) Policy on Humane Care and Use of Laboratory Animals; 3) Guide for the Care and Use of Laboratory Animals (Institute of Laboratory Animal Resources, Commission on Life Sciences, National Research Council, 1996); and 4) AAALACi accreditation. Procedures used in this study have been designed to avoid or minimize unacceptable discomfort, distress, or pain to the animals.

Thirty-nine (39) female athymic nude mice were injected subcutaneously with 10×10⁶ SW620 cells in 0.1 ml PBS with 20% Matrigel on the right flank. When the tumor size reaches within the range of approximately ˜70-200 mm³, mice were sorted as day 0. Thirty (30) mice were randomized into five (5) groups of six (6) mice per group and dosed singly (I.V.) with the test articles or the vehicle control (This day is Day 1: treatment starts). Administrations of Vehicle (saline), NIR dye (“Dye3” in Table 2), Irinotecan, NIR dye-linker-SN38 (XV), and NIR dye-linker Doxo (XVI) are described in Table 6. Before treatment starts/randomization of mice, the 6 mice whose tumors were getting within the range first were used to determine the maximum tolerated dose (MTD) or dose level of two articles NIR day-linker-SN38 (XV), and NIR dye-linker Doxo (XVI), 3 mice for each.

For MTD, the initiated dosage of dye-linker-SN38 is 10 mg/kg and NIR dye-linker Doxo is 10 mg/kg. Mice will be monitored: after dosing immediately, post 1 hr dosing, and post 8 hr dosing, then 24 hr for toxicity including respiratory difficulties, gastrointestinal distress, spastic paralysis, convulsion or blindness, and Piloerection. weight loss, lethargy, discharges, neurological symptoms, and any other signs considered abnormal for animal behavior. Body weight should be included: before dose (day 0: dose starts) and day 1. NIR imaging of those 6 mice: 0.5 hr, 2, 4, 8, and 24 hr post-injection. If there is no toxicity of NIR dye-linker-SN38 (XV) (10 mg/kg), the mice will be dosed again with 15 mg/kg (dose-escalation method to determine MTD).

Tumor size will be assessed daily, and once noticed palpably, the tumor measurements/body weight started daily up to the randomization day, and before randomization, to get 6 mice as mentioned above for the MTD study, and NIR imaging. After randomization, tumor size and body weight were assessed three times a week (M, W, F) for the duration of the study. Monitoring: a), Record BW on M, W, and F; b). Record signs of distress daily; c). Record tumor size M, W, F; d). In vivo NIR imaging (Group 1, 2, 4, and 5): Ex: 780 nm, Em: 810 nm, on day 1:0.5 hr, 2 hr, 4 hr, 8 hr post day 1 dosing, then 24 hr (day 2), then in vivo imaging, every M, W, and F, until the study ended on day 35.

Mice were euthanized on day 35 or mice will be sacrificed when tumor size was more than 2000 mm³. On termination day, NIR imaging of various organs of 3 mice (if the remaining mice >3) from each group 1, 2, 4 and 5. For each mouse: the tumor, heart, liver, spleen, lungs, kidneys, and brain are imaged at the same time.

The tumor-targeting ability and therapeutic efficacy of these DDC prodrugs were studied using two prodrugs (XV and XVI) together with two controls NIR dye, and one chemotherapeutic drugs (Table 7) with the SW620 tumor bearing mice.

TABLE 7
Compounds used in mice studies.

| | XV | XVI |
|---|---|---|
| DDC | Dye 1-SN38 | Dye 1-Doxo |
| Controls | 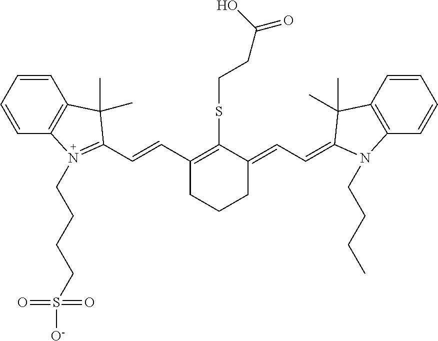 | Irinotecan HCl |
| | Dye 1 | |

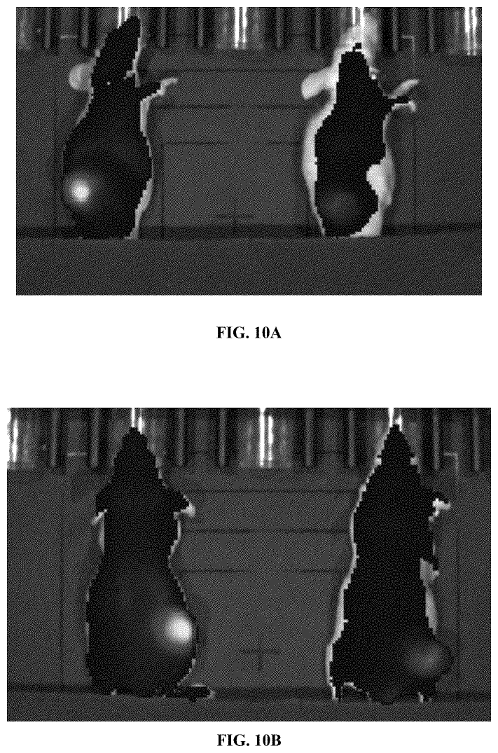

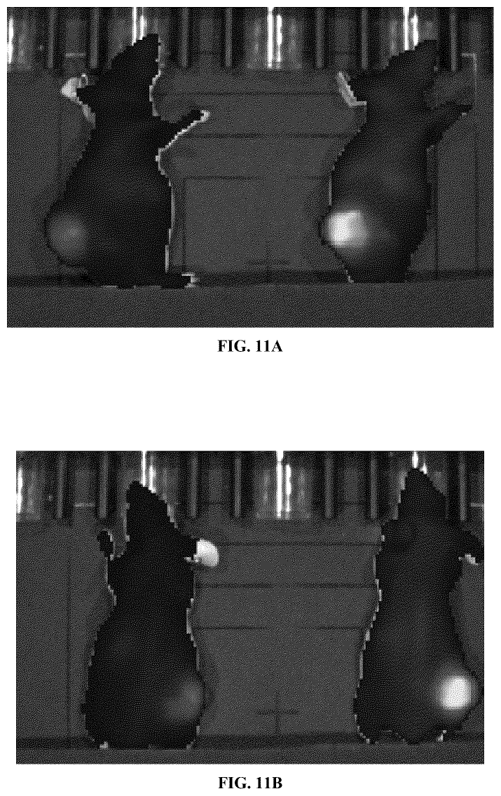

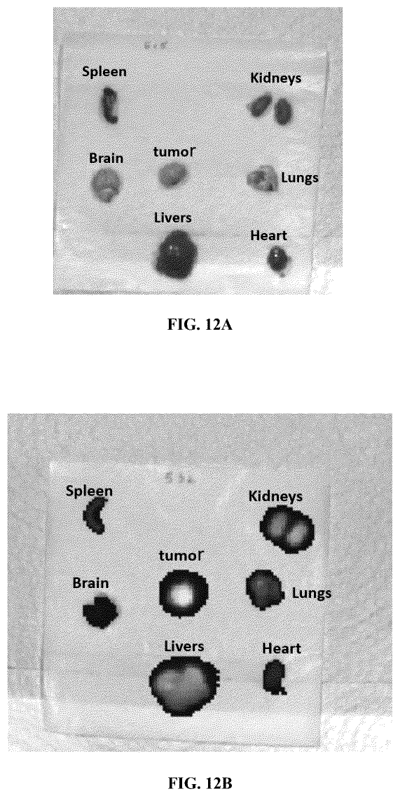

The mice were injected with the said compounds either via intravenous tail injection or with intratumor injection for. Two studies were carried out: one was the compounds uptake study by the tumor as judged by the NIR imaging of both the whole mice body (FIGS. 10A, 10B, 11A, and 11B) and of the mice organs (FIGS. 12A, 12B, 13A, 13B, 14A and 14B). The injection method for these study was via intravenous tail injection. The second was the efficacy evaluation of these compounds as judged by their tumor growth inhibition, the injection method for these study was via both intravenous tail injection and intratumor injection.

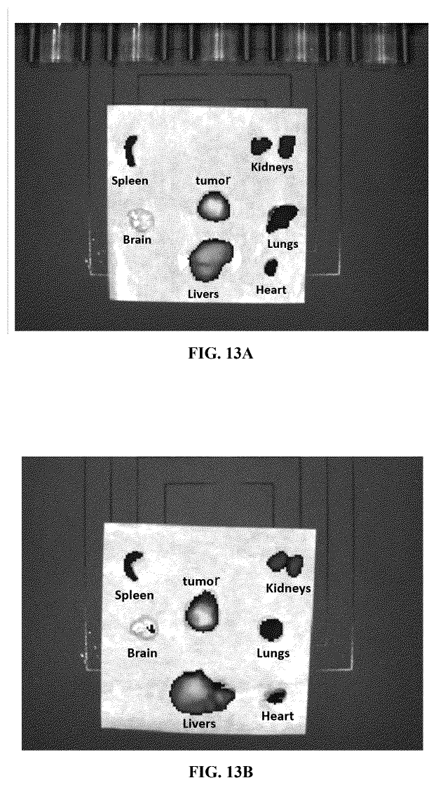

Uptake study by the tumor as judged by the NIR imaging. FIGS. 10A and 10B shows the whole body NIR imaging of mice bearing SW620 tumor bearing mice with intravenous tail injection with Compound XV (NIR Dye-SN38) 11 days after the second dosing at 15 mg/kg with 10 seconds exposure. FIG. 10A is the NIR imaging of tumor site; and FIG. 10B is the NIR imaging of dorsal site. FIGS. 11A and 11B shows the whole body NIR imaging of mice bearing SW620 tumor bearing mice with intravenous tail injection with Compound XVI (NIR Dye-Doxo) 11 days after the second dosing at 15 mg/kg with 10 seconds exposure. FIG. 11A is the NIR imaging of tumor site; and FIG. 11B is the NIR imaging of dorsal site. These results demonstrate that both test compounds (XV and XVI) accumulate selectively in the tumor sites as judged by the distinct bright NIR fluorescent signals The NIR fluorescence imaging for individual organs were also studied after mice were sacrificed. FIG. 12A is the NIR imaging of organs from a mouse bearing SW620 tumor with vehicle (50% DMSO: 40% PEG400: 10% ethanol) after 24 hours. FIG. 12B is the NIR imaging of organs from a mouse bearing SW620 tumor with Compound XV with 2^{nd} dose of 15 mg/kg after 24 hr intravenous tail injection-Auto exposure. There was no NIR fluorescent activity in FIG. 12A for the control mouse with the vehicle injection However, NIR fluorescent signals in FIG. 12B from the organs of the mouse injected with the DDC compound showed an accumulation of signal. In addition, the tumor showed the strongest NIR fluorescent signal among all the organs, indicating the DDC compound selectively localized in the tumor. FIG. 13A is the NIR imaging of organs from a mouse bearing SW620 tumor with Compound XVI from first dose of 10 mg/kg day 17 after intravenous tail injection with 10 s exposure. FIG. 13B is the NIR imaging of organs from a mouse bearing SW620 tumor with Compound XVI a second dose of 15 mg/kg day 7 after intravenous tail injection with 10 s exposure.

The strong NIR fluorescent signals present in FIG. 13A in the tumor 17 days after the first dose injection indicates that the DDC compound persists in the tumor over a long period of time. Not surprisingly, the NIR fluorescence is more intense in the tumor 7 days after the second injection shown in FIG. 13B.

The tumor uptake properties of the NIR dye that is the precursor to the final prodrugs were also studied similarly.

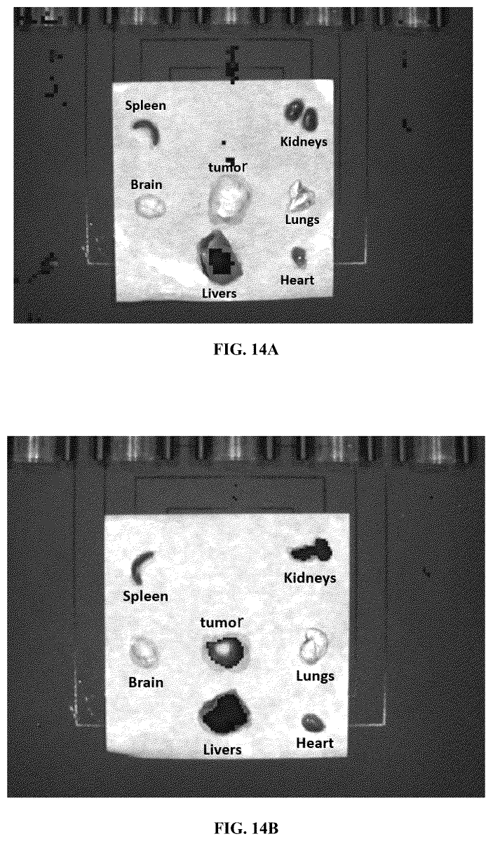

FIG. 14A is the NIR imaging of organs from a mouse bearing SW620 tumor with the control NIR dye from Table 6 from first dose of 4.37 mg/kg day 17 after intravenous tail injection with 10 s exposure.

FIG. 14B is the NIR imaging of organs from a mouse bearing SW620 tumor with the control NIR dye from Table 6 from with second dose of 4.37 mg/kg day 7 after intravenous tail injection with 10 s exposure.

Interestingly, there are no NIR signals from this control group 17 days after the first injection (FIG. 14A), as compared with the strong NIR signals for the mice injected with the DDC conjugate compound. The strong NIR signals were observed from the mouse 7 days after with the second dose. It is safe to say that the tumor targeting properties of the NIR dye does not get lost in the conjugate, rather it was maintained as conjugates.

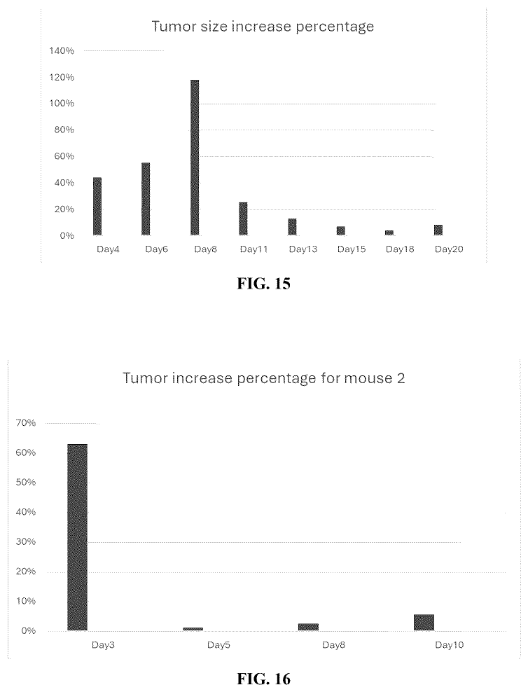

FIG. 15 and Table 9 shows the tumor size changes of Dye 1-SN38 (XV) with 10 mg/kg first dose, followed by a second dose 15 mg/kg on Day 11 via intravenous tail injection. Since the tumor growth did not seem to slow down in the first 10 days after the first intravenous tail injection, a second dose was applied. After the second dose, the tumor growth rate dropped significantly from 63% to a low 4%.

TABLE 8
Tumor size changes of Dye 1-SN38 (XV).

| Day | Day 1 | Day 4 | Day 6 | Day 8 | Day 11 | Day 13 | Day 15 | Day 18 | Day 20 |
|---|---|---|---|---|---|---|---|---|---|
| Tumor size | 163.7 | 235.4 | 364.9 | 794.5 | 988.8 | 11114.9 | 1193.3 | 1239.1 | 1342.1 |

| | | | | | | | | |
|---|---|---|---|---|---|---|---|---|
| Tumor increase rate | 44% | 55% | 118% | 25% | 12.7% | 7% | 3.8% | 8.3% |

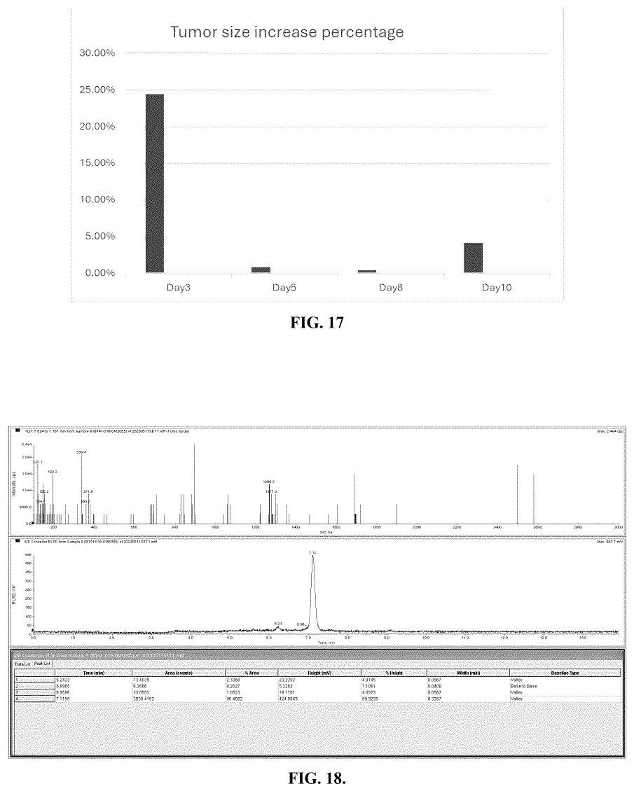

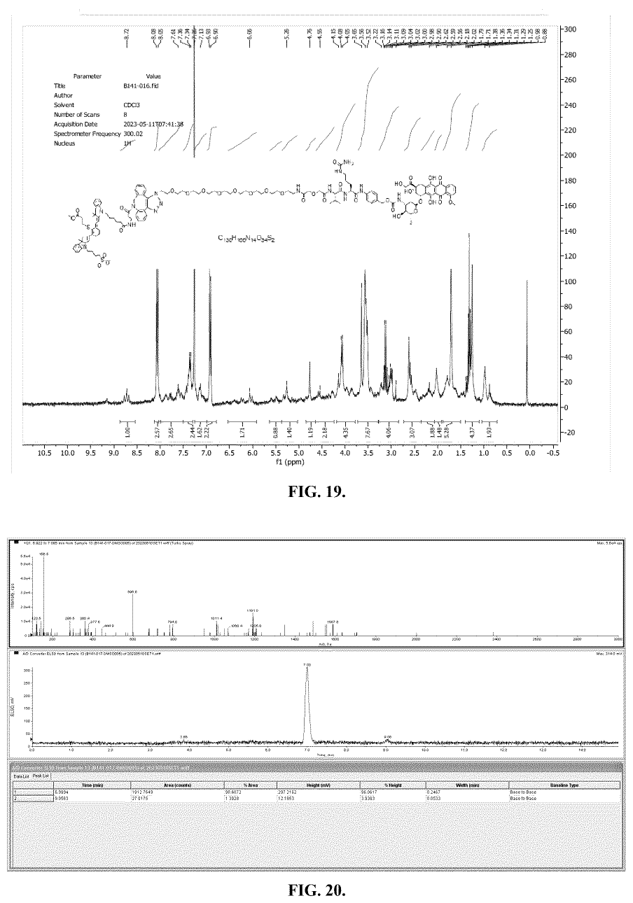

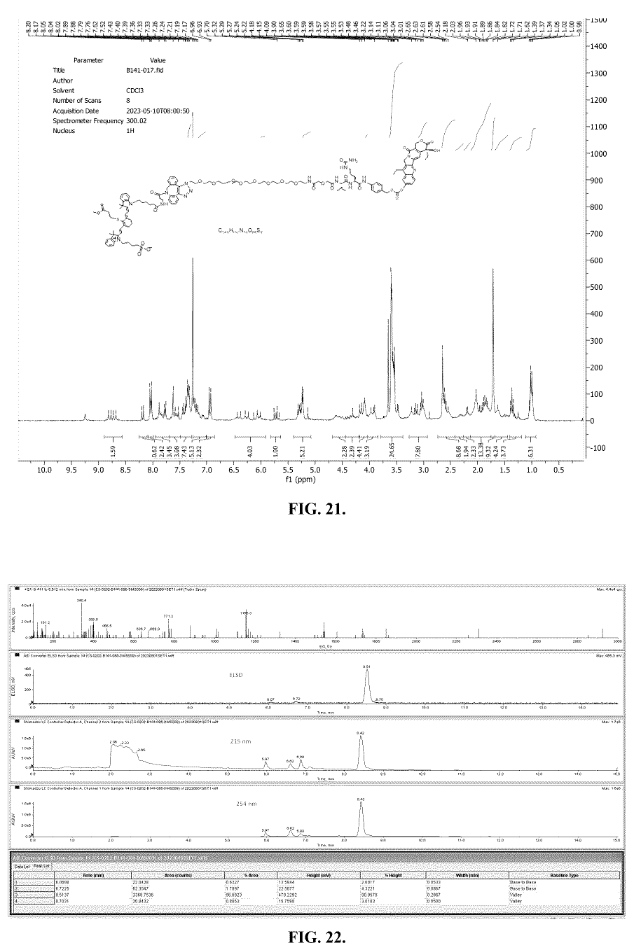

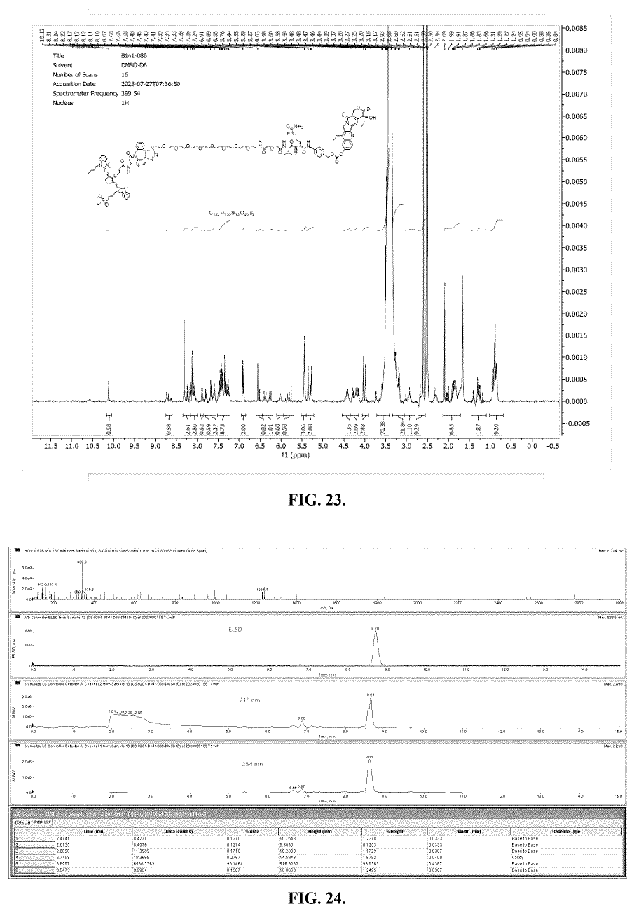

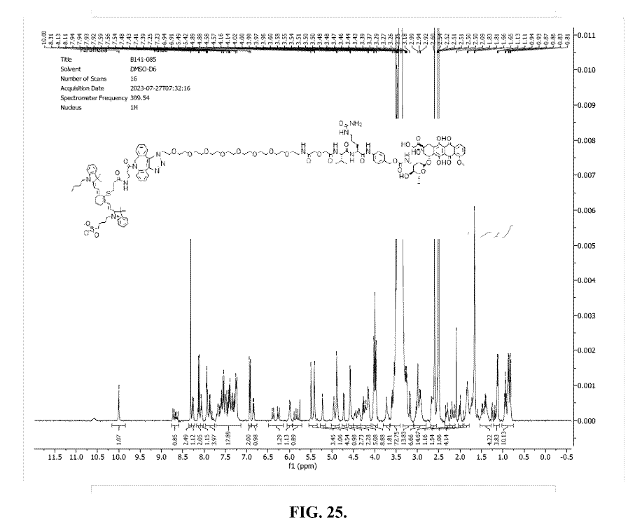

FIGS. 16 and 17 and Tables 9 and 10, respectively, show the tumor changes of Dye 1-SN38 (XV) and Dye 1-Doxo (XVI) via intratumor injection at 15 mg/kg dose. The results indicated that for mice injected with both DDC conjugate compounds, the tumor growth rates slowed significantly from 63% and 24% to as low as 1% and 0.4% respectively for XV and XVI.

TABLE 9
Tumor size (mm³) changes of Dye 1-SN38 (XV).

| Day | Day 1 | Day 4 | Day 6 | Day 8 | Day 11 | Day 13 | Day 15 | Day 18 | Day 20 |
|---|---|---|---|---|---|---|---|---|---|
| Tumor size | 163.7 | 235.4 | 364.9 | 794.5 | 988.8 | 11114.9 | 1193.3 | 1239.1 | 1342.1 |

| | | | | | | | | |
|---|---|---|---|---|---|---|---|---|
| Tumor increase rate | 44% | 55% | 118% | 25% | 12.7% | 7% | 3.8% | 8.3% |

TABLE 10
Tumor size (mm³) changes of Dye 1-Doxo (XVI).

| Day | Day 1 | Day 3 | Day 5 | Day 8 | Day 10 |
|---|---|---|---|---|---|
| Tumor size | 855.4 | 1393.5 | 1409 | 1443 | 1523.7 |

| | | | | |
|---|---|---|---|---|
| Tumor increase rate | 63% | 1.1% | 2.4% | 5.5% |

The particular embodiments or elements of the invention disclosed by the description or shown in the figures, structures, schemes, or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures found herein.

What is claimed is:

1. A composition comprising:
a conjugate comprising:
a tumor-targeting near-infrared dye; and
a therapeutic agent;
wherein said near-infrared dye targets said therapeutic agent to tumor cells.

2. The composition of claim 1, wherein said near-infrared dye comprises:

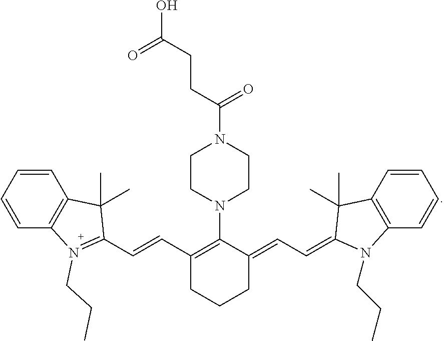

3. The composition of claim 1, wherein said near-infrared dye comprises:

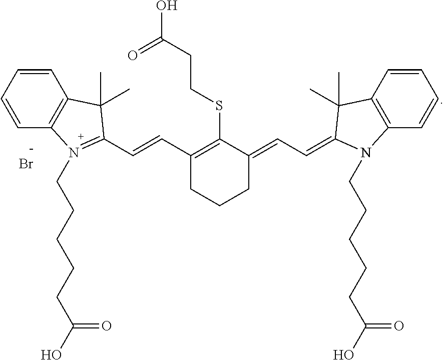

4. The composition of claim 1, wherein said near-infrared dye comprises:

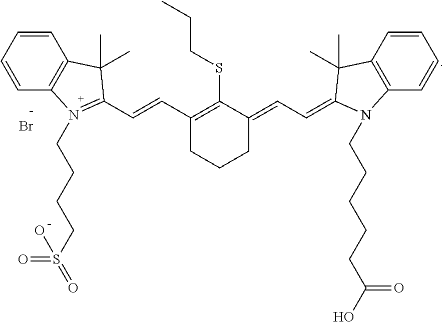

5. The composition of claim 1, wherein said near-infrared dye comprises:

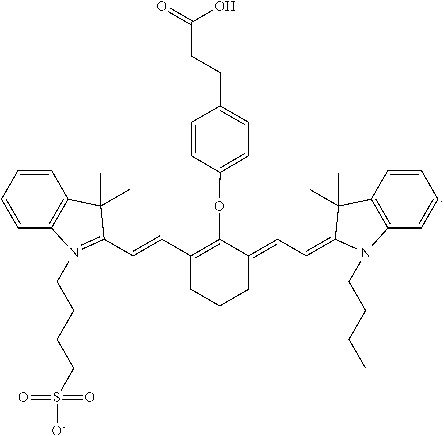

6. The composition of claim 1, wherein said near-infrared dye comprises:

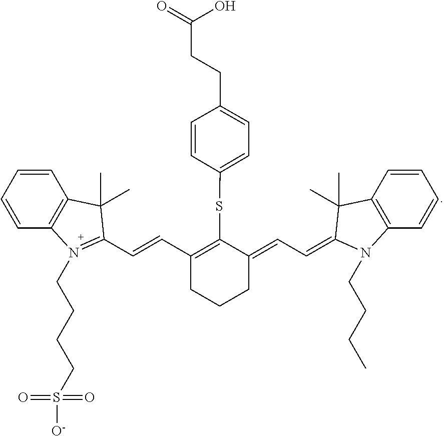

7. The composition of claim 1, wherein said near-infrared dye comprises a zwitterion.

8. The composition of claim 7, wherein said zwitterion comprises imidazolium sulfonate.

9. The composition of claim 7, wherein said zwitterion comprises guanidinium carboxylate.

10. The composition of claim 1, wherein said near-infrared dye and said therapeutic agent are covalently bound via a linker.

11. The composition of claim 10, wherein said linker comprises glycine-phenylalanine-leucine-glycine.

12. The composition of claim 10, wherein said linker comprises glycine-glycine-phenylalanine-glycine.

13. The composition of claim 1, wherein said conjugate comprises:

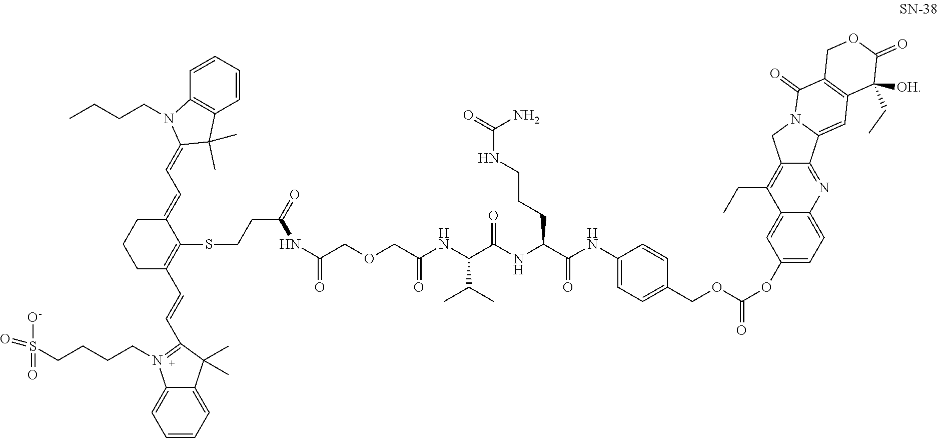

14. The composition of claim 1, wherein said conjugate comprises:

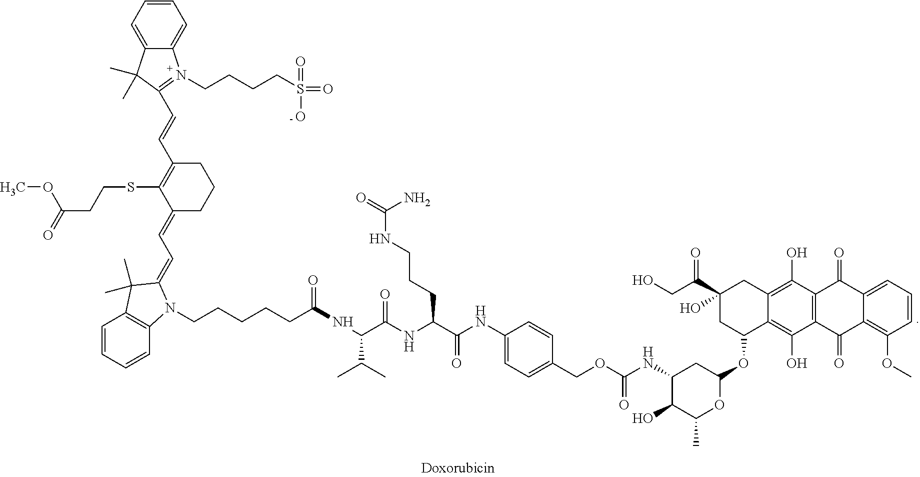

15. The composition of claim 1, wherein said conjugate comprises:

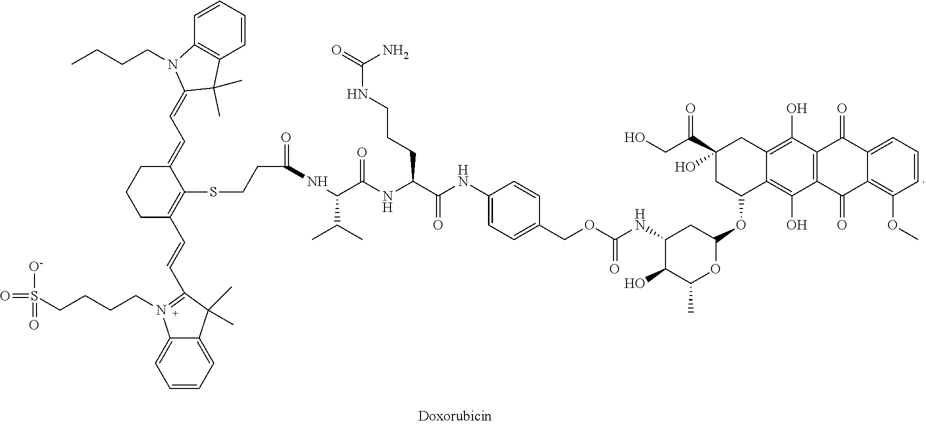

16. The composition of claim 1, wherein said conjugate comprises:

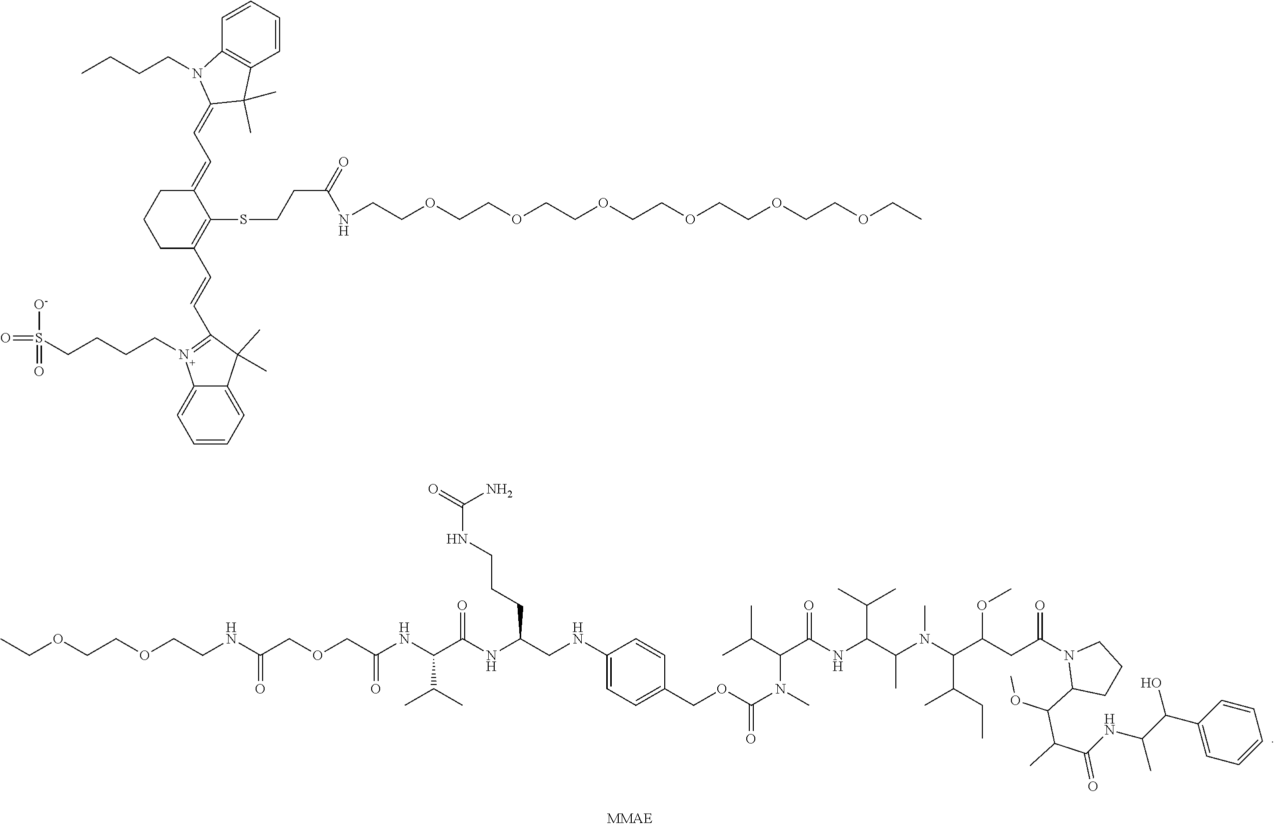

17. The composition of claim 1, wherein said conjugate comprises:

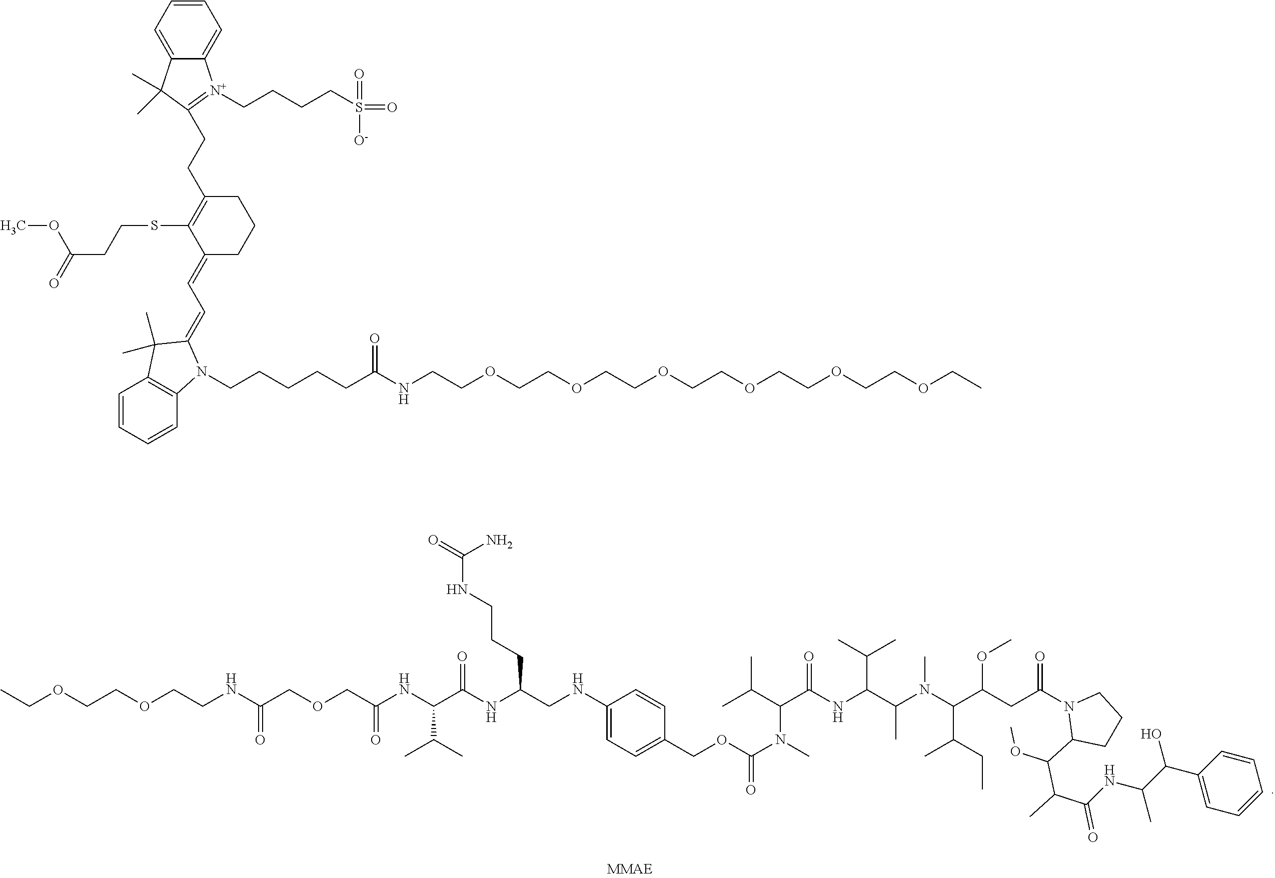

18. The composition of claim 1, wherein said conjugate comprises:

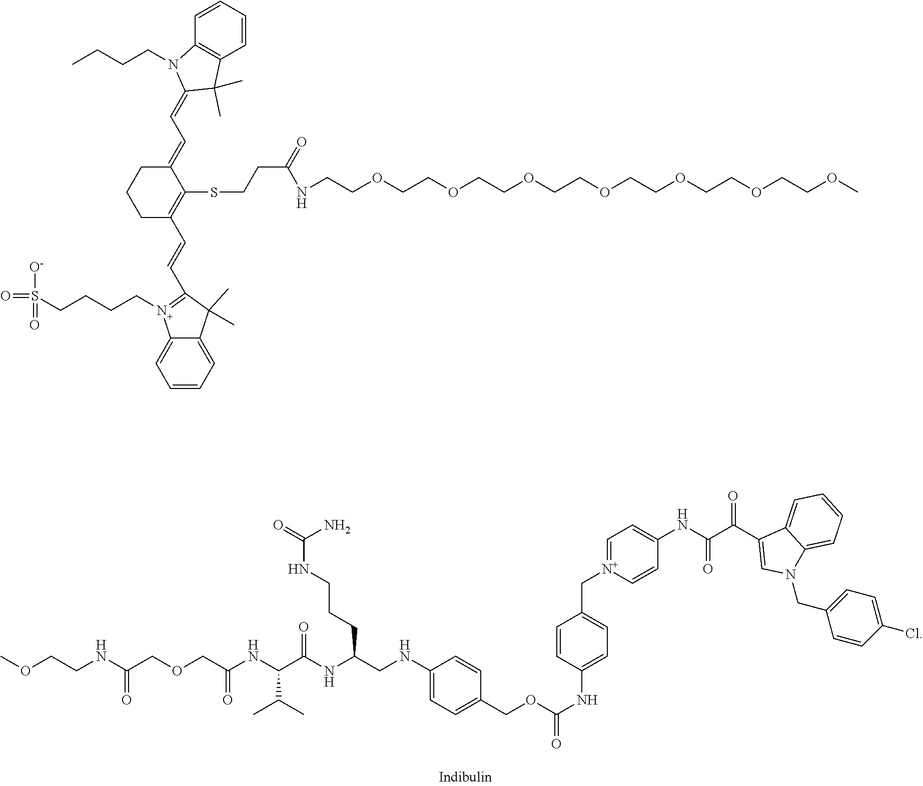

19. The composition of claim 1, wherein said conjugate comprises:

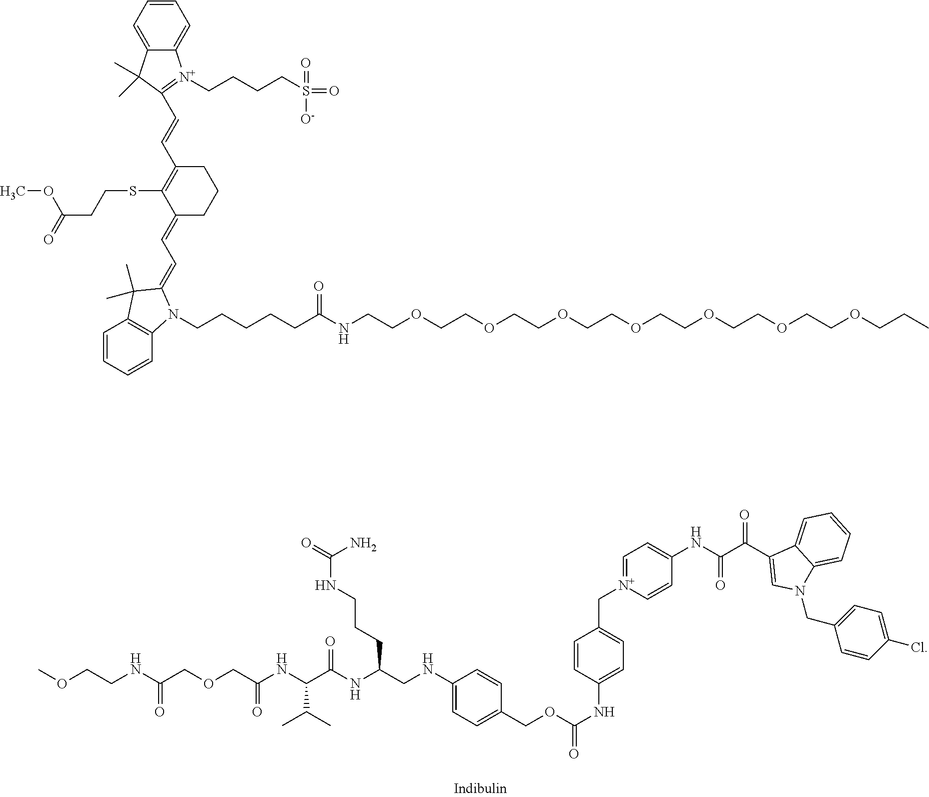

20. The composition of claim 1, wherein said conjugate comprises:

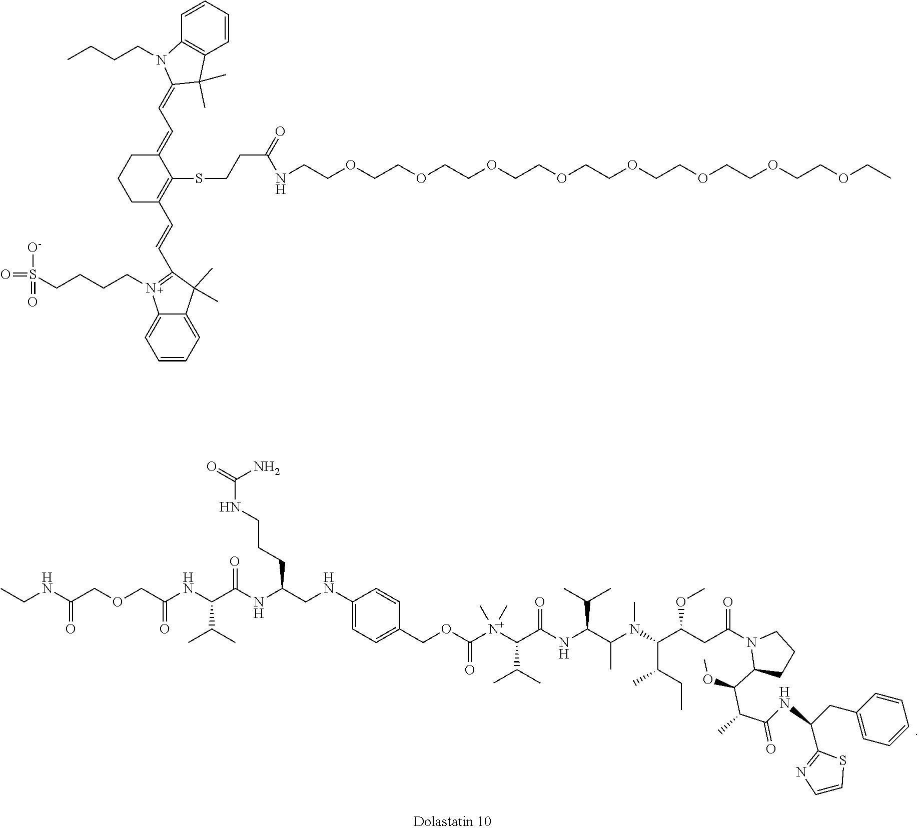

21. The composition of claim 1, wherein said conjugate comprises:

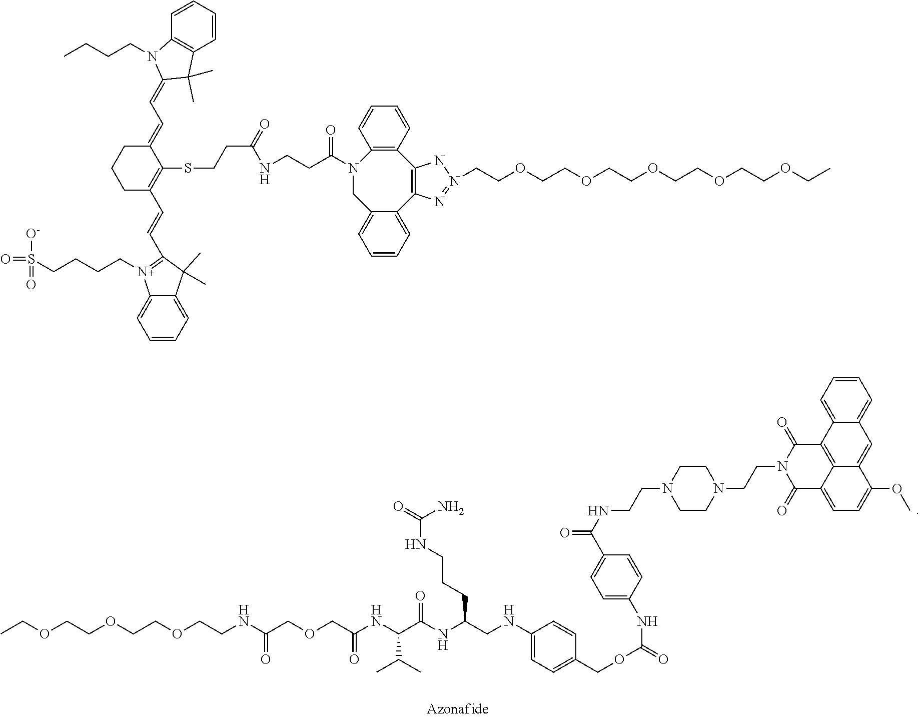

22. The composition of claim 1, wherein said conjugate comprises:

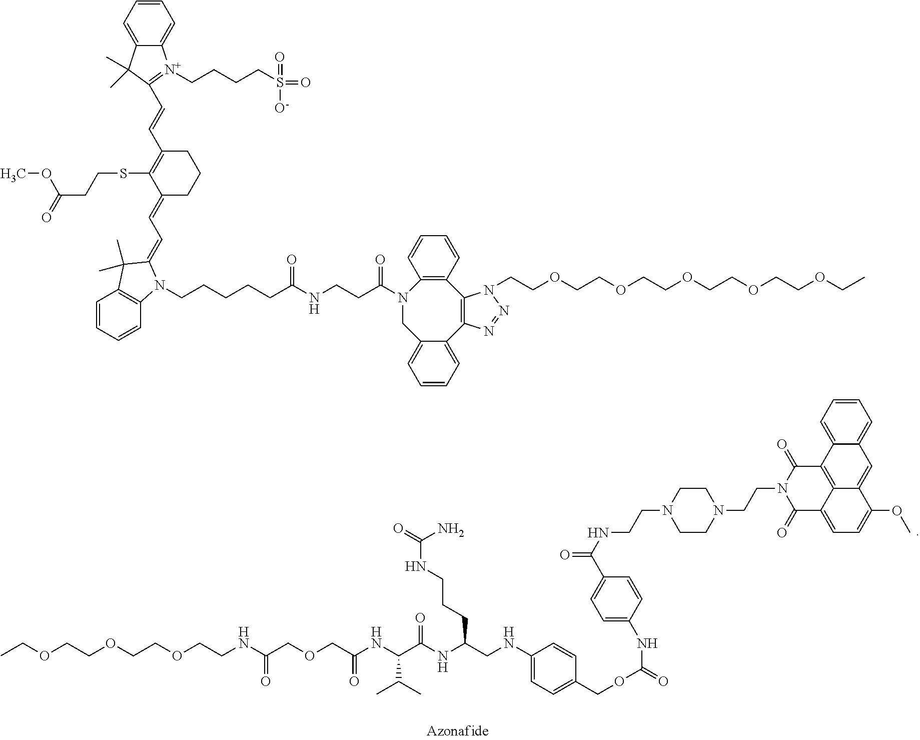

23. The composition of claim 1, wherein said conjugate comprises:

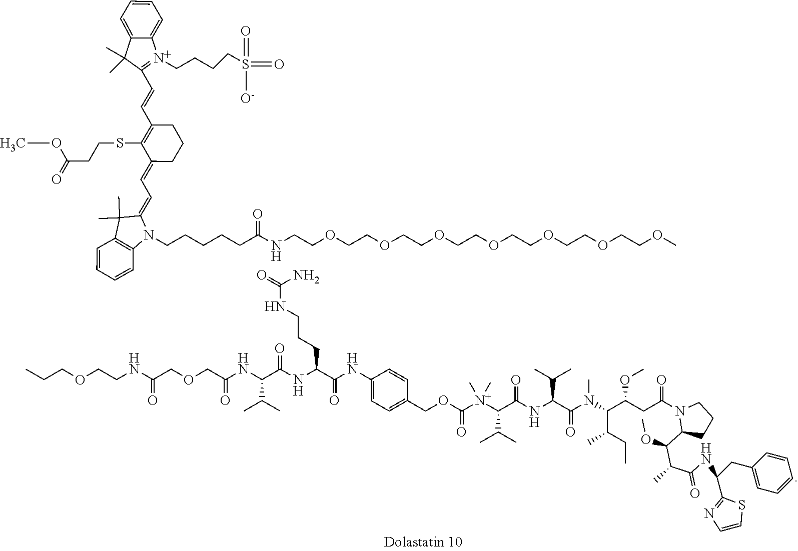

24. The composition of claim 1, wherein said conjugate comprises:

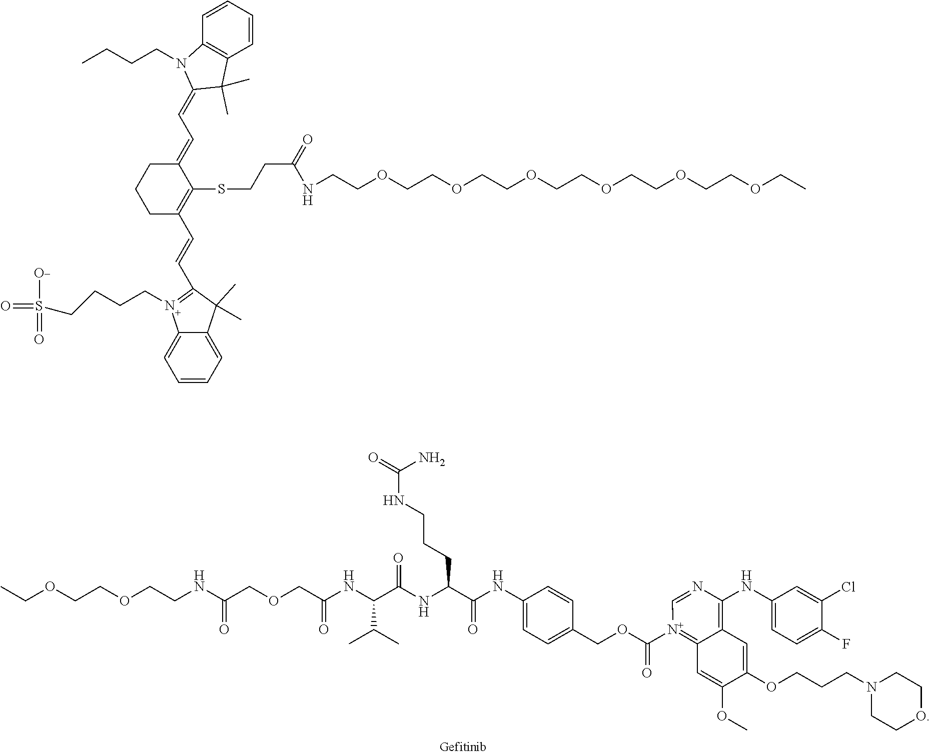

25. The composition of claim 1, wherein said conjugate comprises:

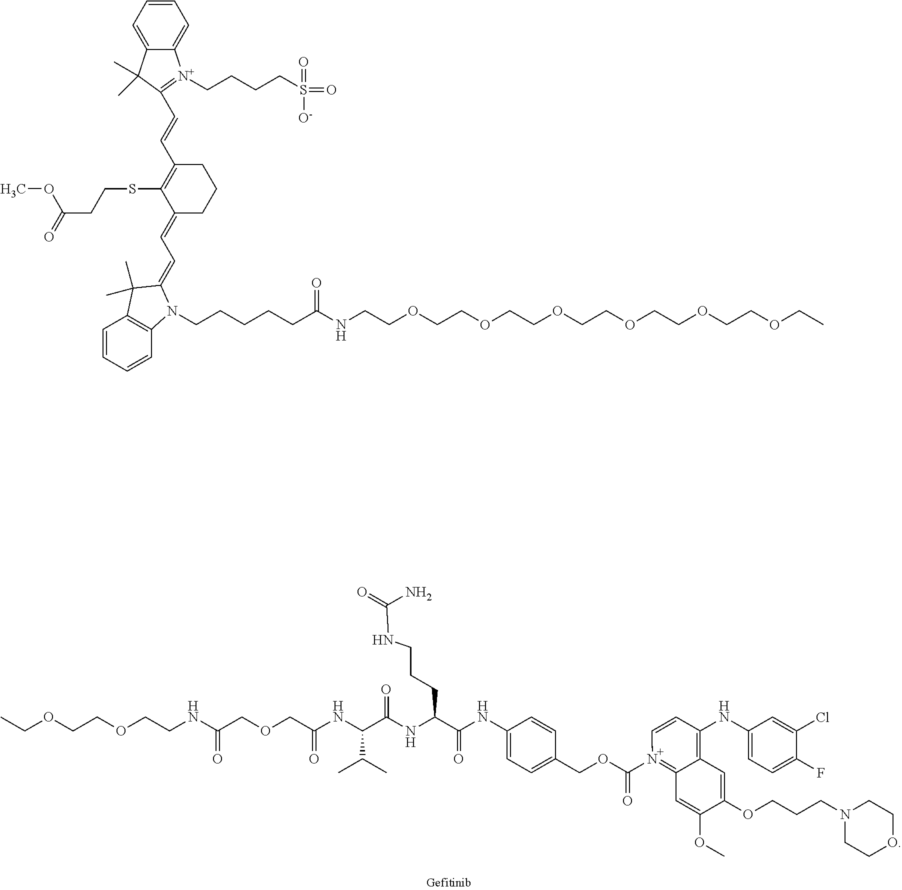

26. The composition of claim 1, wherein said conjugate comprises:

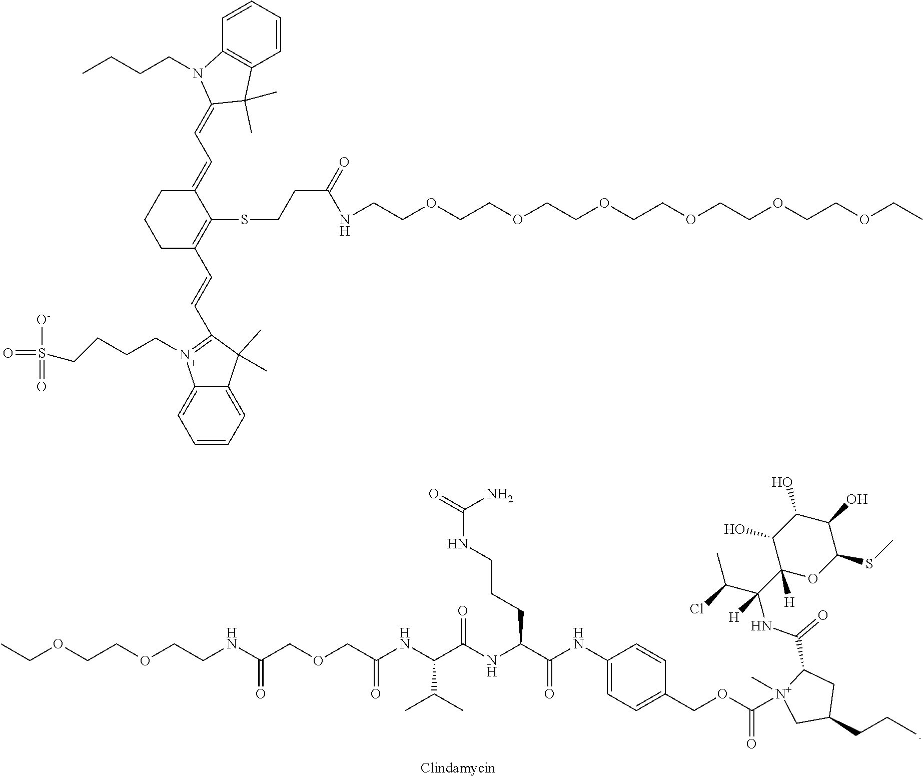

27. The composition of claim 1, wherein said conjugate comprises:

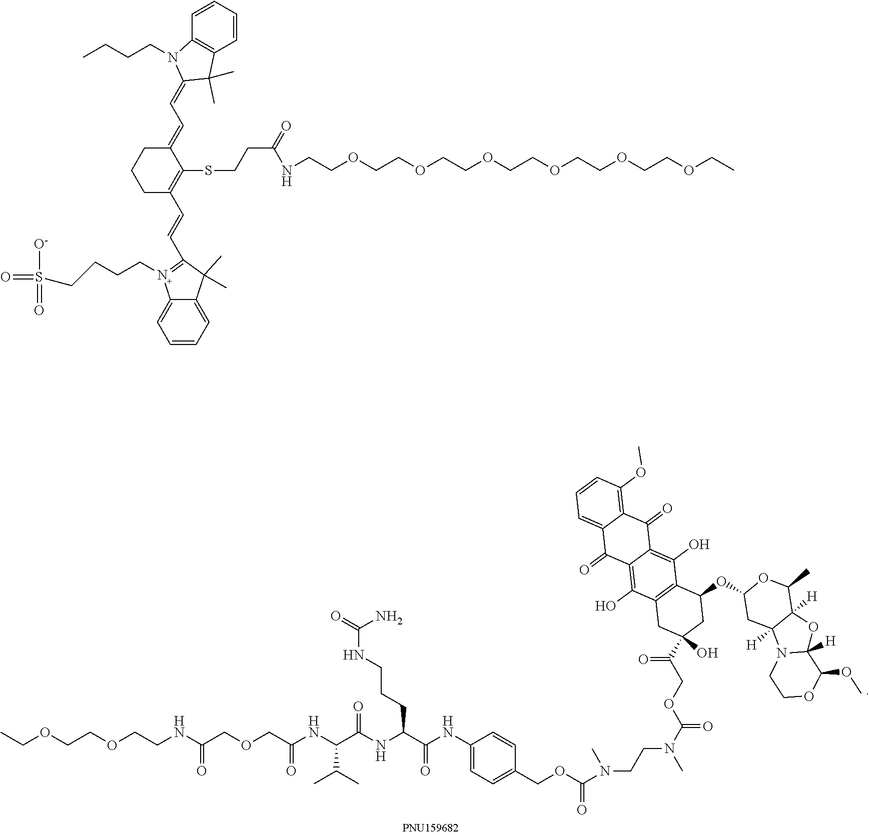

28. The composition of claim 1, wherein said conjugate comprises:

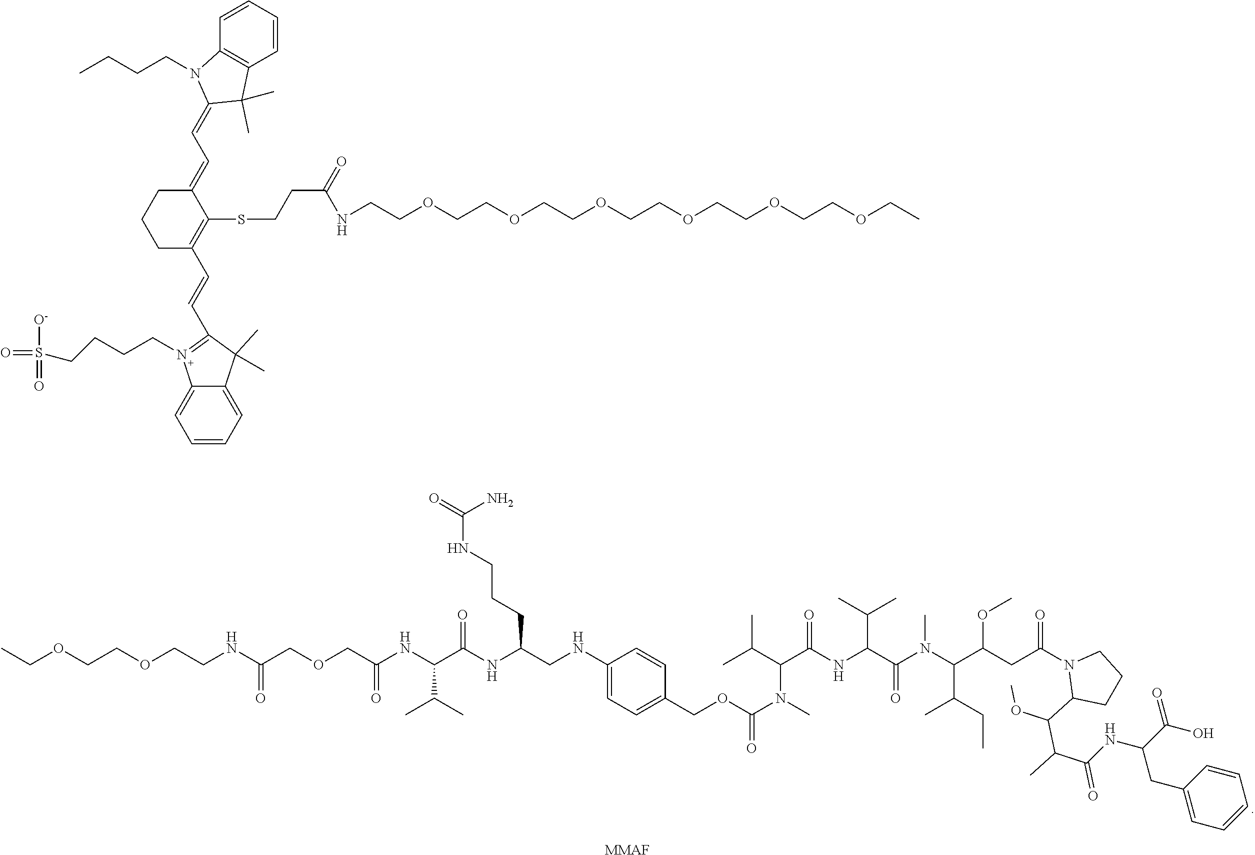

29. The composition of claim 1, wherein said conjugate comprises:

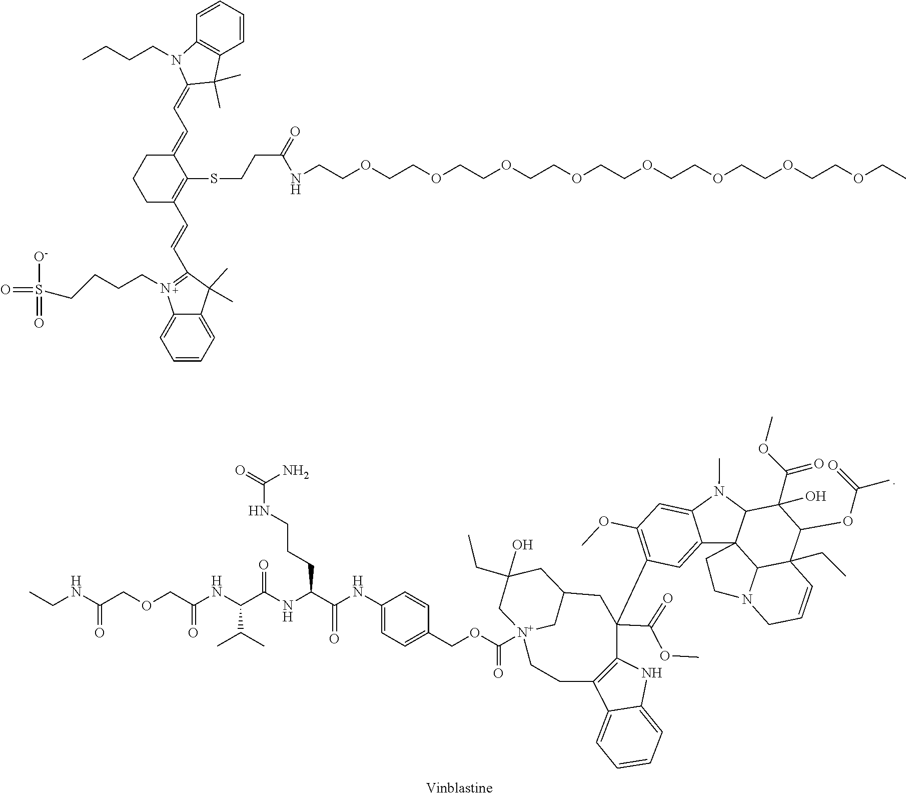

30. The composition of claim 1, wherein said conjugate comprises:

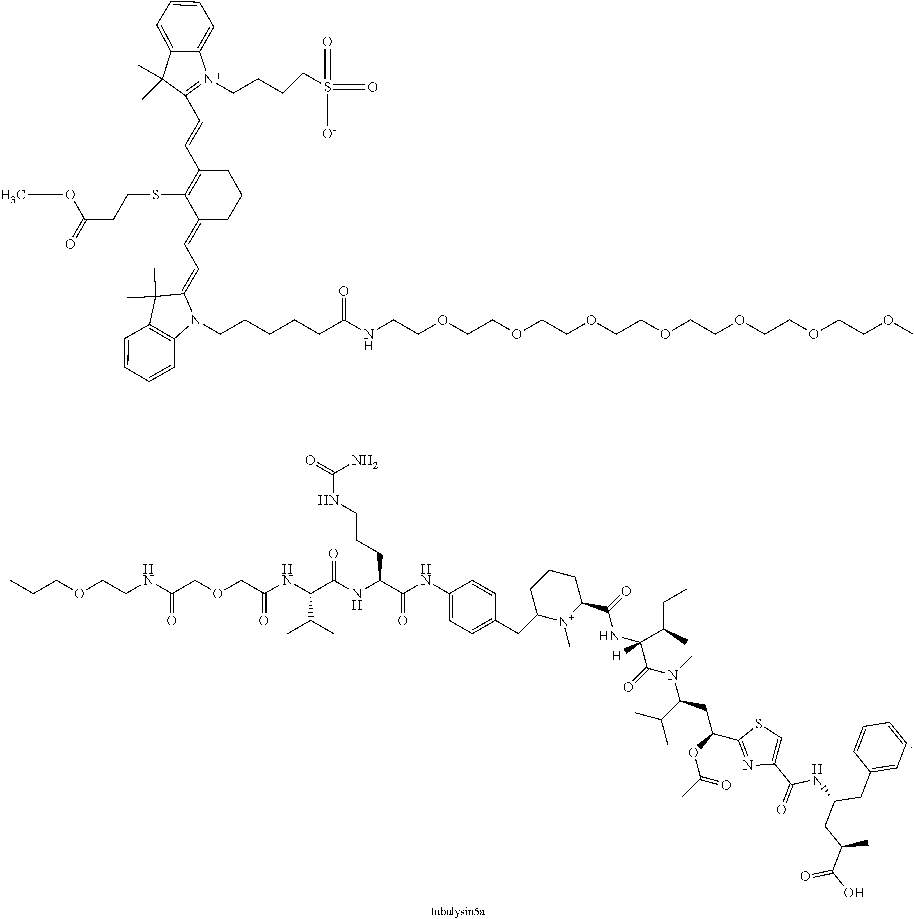

31. The composition of claim 1, wherein said conjugate comprises:

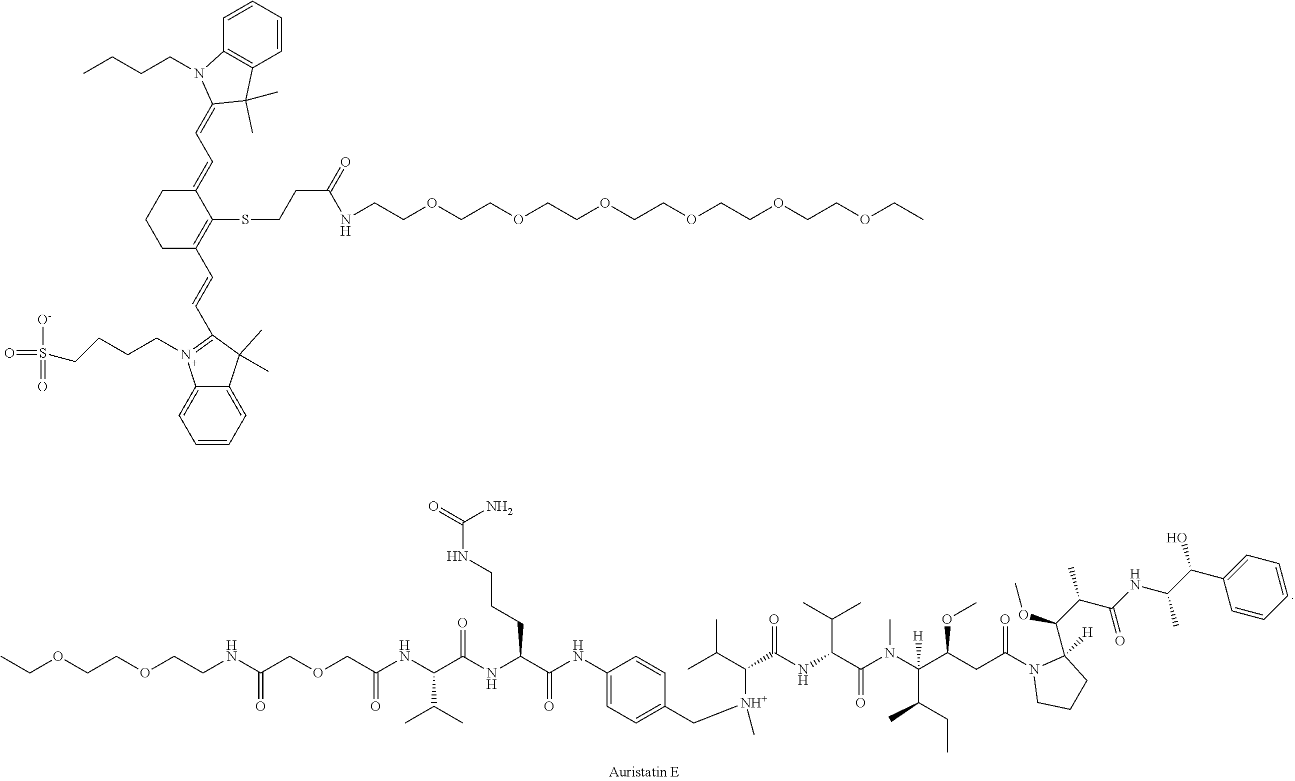

32. The composition of claim 1, wherein said conjugate comprises:

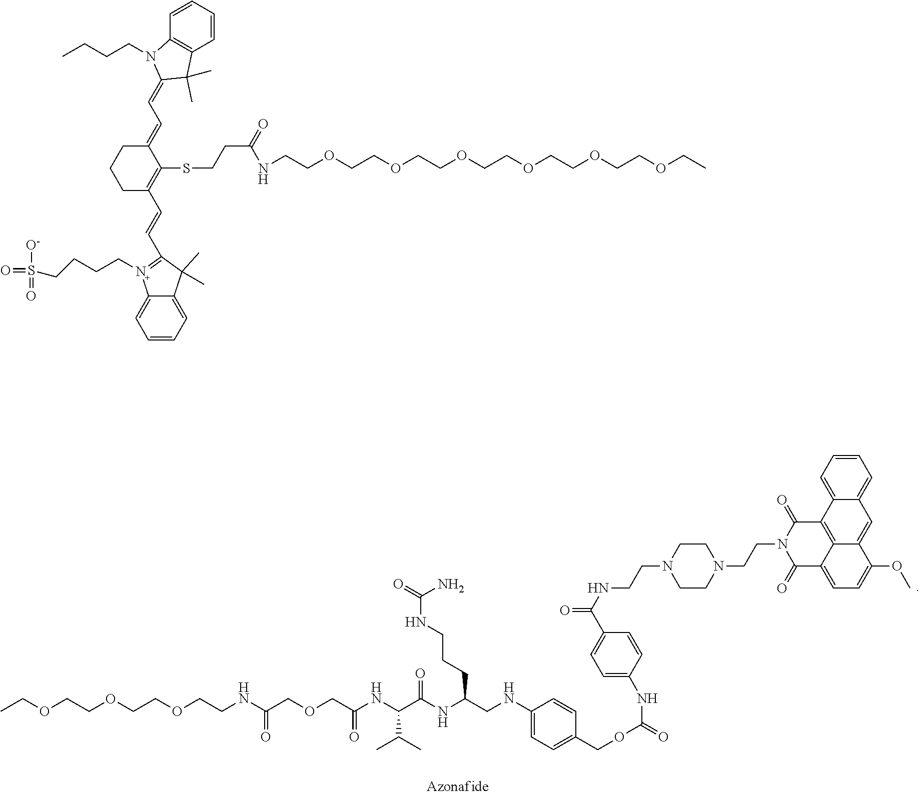

33. The composition of claim 1, wherein said conjugate comprises:

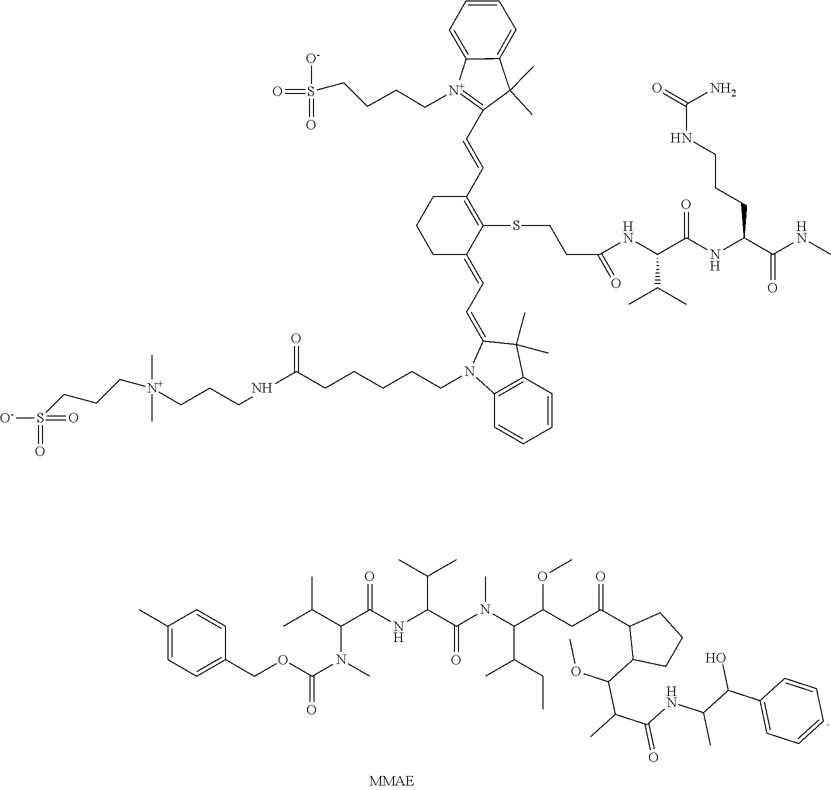

34. The composition of claim 1, wherein said conjugate comprises:

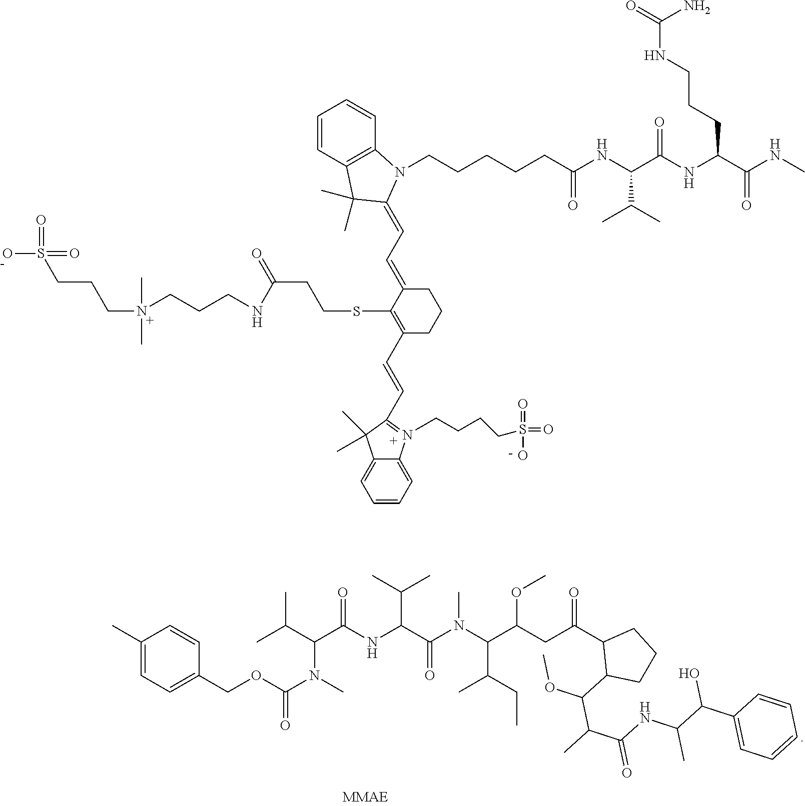

35. The composition of claim 1, wherein said conjugate comprises:

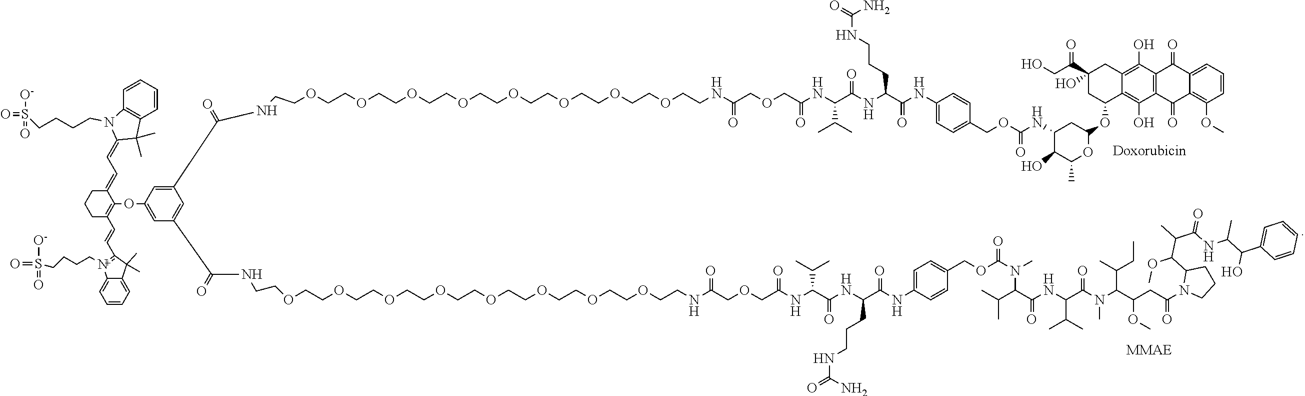

36. The composition of claim 1, wherein said conjugate comprises:

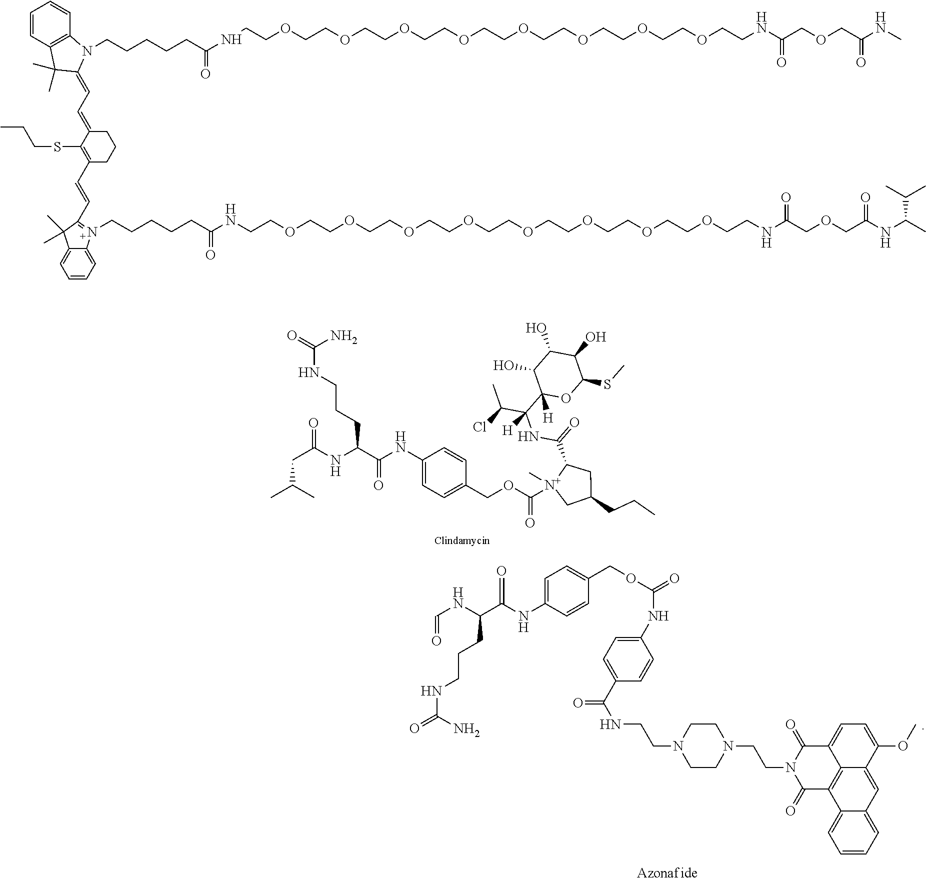

37. The composition of claim 1, wherein said conjugate comprises: [chemical structure]
